(19) United States
(12) Patent Application Publication
(10) Pub. No.: US 2023/0257364 A1
(43) Pub. Date: Aug. 17, 2023

(54) PYRIDAZINONE-BASED COMPOUNDS AS AXL, C-MET, AND MER INHIBITORS AND METHODS OF USE THEREOF

(71) Applicants: CMG Pharmaceutical Co., Ltd., Seongnam (KR); Sungkwang Medical Foundation, Seoul (KR)

(72) Inventors: Jin Sung KIM, Seoul (KR); Sungmoo KIM, Seongnam (KR); Ju Hui JEONG, Seongnam (KR); Hyun Sook AN, Seongnam (KR); Soojeong KIM, Yongin (KR)

(73) Assignees: CMG Pharmaceutical Co., Ltd., Seongnam (KR); Sungkwang Medical Foundation, Seoul (KR)

(21) Appl. No.: 18/161,609

(22) Filed: Jan. 30, 2023

Related U.S. Application Data
(60) Provisional application No. 63/310,823, filed on Feb. 16, 2022.

(51) Int. Cl. C07D 401/12; C07D 401/14

(52) U.S. Cl. CPC C07D 401/12; C07D 401/14

(57) ABSTRACT

Provided is an inhibitor of AXL, Mer, and/or c-Met of Formula (I) or a pharmaceutically acceptable salt thereof:

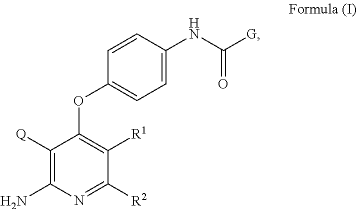

in which R¹, R², R³, G, and Q are described herein. Further provided is a method of treating or preventing an AXL-, Mer-, and/or c-Met-mediated disease using an effective amount of the compound of Formula (I) or a pharmaceutically acceptable salt thereof. When AXL, MER, and/or c-Met is inhibited, the compound or pharmaceutically acceptable salt thereof can re-sensitize cancer cells, such as non-small cell lung cancer cells, that have grown resistant to an anti-cancer agent.

### CROSS-REFERENCE TO PRIOR APPLICATIONS

This application claims benefit to U.S. Provisional Patent Application No. 63/310,823, filed Feb. 16, 2022, which is hereby incorporated by reference in its entirety.

### BACKGROUND OF THE INVENTION

Receptor tyrosine kinases (RTKs) are transmembrane proteins that transduce signals from the extracellular environment to the cytoplasm and nucleus to regulate normal cellular processes, including survival, growth, differentiation, adhesion, and mobility. Over expression or activation of RTKs has been implicated in the pathogenesis of various cancers, linked with cell transformation, tumor formation, and metastasis.

TAM receptors are expressed in various cells and tissues. AXL is a member of the TAM RTK family, which also includes TYR03 and Mer, originally identified as a transforming gene expressed in cells from patients with chronic myelogenous leukemia (O'Bryan et al., Mol. Cell Biol., 1991, 11, 5016-5031) and chronic myeloproliferative disorder (Janssen et al., Oncogene, 1991, 6(11), 2113-2120). AXL contributes to at least three of the six fundamental mechanisms of malignancy in cancer, by promoting cancer cell migration and invasion, involving in tumor angiogenesis, and facilitating cancer cell survival and tumor growth (Holland et al., Cancer Res., 2005, 65(20), 9294-9303; Tai et al., Oncogene, 2008, 27, 4044-4055; Li et al., Oncogene, 2009, 28, 3442-3455; and Mudduluru et al., Mol. Cancer Res., 2010, 8(2), 159-169).

In addition, over expression of AXL also has been implicated in asthma, pain, and dermatitis (Shibata et al., J Immunol, 2014, 192(8), 3569-3581; Liang et al., Molecular Pain, 2020, 16, 1-13; and Bauer et al., J Exp Med, 2012, 209(11), 2033-2047).

Over expression of c-MET is associated with the development and poor prognosis of a wide range of solid tumors, including breast, prostate, thyroid, lung, stomach, colorectal, pancreatic, kidney, ovarian, and uterine carcinoma, malignant glioma, uveal melanoma, and osteo- and soft-tissue sarcoma (Jiang et al., Critical Reviews in Oncology Hematology, 2005, 53(1), 35-69). a

Given the roles of AXL, Mer, and c-MET in a variety of diseases, there remains a need for the development of agents that act as inhibitors of AXL, Mer, and/or C-Met to therapeutically treat such diseases.

### BRIEF SUMMARY OF THE INVENTION

The present invention provides a compound of Formula (I) or a pharmaceutically acceptable salt thereof:

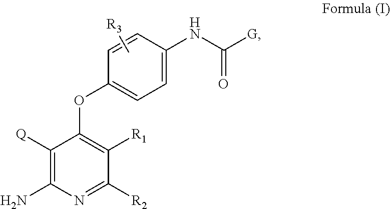

in which R¹, R², R³, G, and Q are as described herein.

The invention further provides a method of treating or preventing an AXL-, Mer-, and/or c-Met-mediated disease in a subject comprising administering to the subject an effective amount of the compound of Formula (I) or a pharmaceutically acceptable salt thereof.

The invention provides a method of inhibiting an AXL, Mer, and/or c-Met enzyme in a cell comprising administering a compound of Formula (I) or a pharmaceutically acceptable salt thereof to the cell.

### BRIEF DESCRIPTION OF THE SEVERAL VIEWS OF THE DRAWING(S)

FIG. 1 is a chemical synthesis of N-(4-(2-amino-3-chloropyridin-4-yloxy)-3-fluorophenyl)-2-(4-fluorophenyl)-3-oxo-2,3-dihydropyridazine-4-carboxamide in an aspect of the invention.

FIG. 2 is a chemical synthesis of N-(4-(2-amino-3-(3-morpholino-3-oxoprop-1-ynyl)pyridin-4-yloxy)-3-fluorophenyl)-2-(4-fluorophenyl)-3-oxo-2,3-dihydropyridazine-4-carboxamide in an aspect of the invention.

FIG. 3 is a chemical synthesis of (E)-N-(4-(2-amino-3-(3-morpholino-3-oxoprop-1-enyl)pyridine-4-yloxy)-3-fluorophenyl)-2-(4-fluorophenyl)-3-oxo-2,3-dihydropyridazine-4-carboxamide in an aspect of the invention.

FIG. 4 is a chemical synthesis of N-(4-(2-amino-3-(3-cyanopyridin-4-yloxy)-3-fluorophenyl)-2-(4-fluorophenyl)-3-oxo-2,3-dihydropyridazine-4-carboxamide in an aspect of the invention.

FIG. 5 is a chemical synthesis of N-(4-(2-amino-3-(3-(piperazin-1-yl)prop-1-ynyl)pyridin-4-yloxy)-3-fluorophenyl)-2-(4-fluorophenyl)-3-oxo-2,3-dihydropyridazine-4-carboxamide in an aspect of the invention.

FIG. 6 is a chemical synthesis of (E)-N-(4-(2-amino-3-(3-oxo-3-(piperazin-1-yl)prop-1-enyl)pyridin-4-yloxy)-3-fluorophenyl)-2-(4-fluorophenyl)-3-oxo-2,3-dihydropyridazine-4-carboxamide in an aspect of the invention.

FIG. 7 is a chemical synthesis of N-(4-(2-amino-3-(4-morpholinobut-1-ynyl)pyridin-4-yloxy)-3-fluorophenyl)-2-(4-fluorophenyl)-3-oxo-2,3-dihydropyridazine-4-carboxamide in an aspect of the invention.

FIG. 8 is a chemical synthesis of N-(4-(2-amino-3-(3-(4-hydroxypiperidin-1-yl)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention.

FIG. 9 is a chemical synthesis of N-(4-(2-amino-3-(3-(4-methoxypiperidin-1-yl)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention.

FIG. 10 is a chemical synthesis of N-(4-(2-amino-3-(3-(2-methoxyethoxyamino)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention.

FIG. 11 is a chemical synthesis of N-(4-(2-amino-3-(3-((2-methoxyethoxy)(methyl)amino)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention.

FIG. 12 is a chemical synthesis of N-(4-(2-amino-3-(3-oxo-3-(piperazin-1-yl)prop-1-ynyl)pyridin-4-yloxy)-3-fluorophenyl)-2-(4-fluorophenyl)-3-oxo-2,3-dihydropyridazine-4-carboxamide in an aspect of the invention.

FIG. 13 is a chemical synthesis of N-(4-(2-amino-3-(3-(4-(2-methoxyethyl)piperazin-1-yl)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention.

FIG. 14 is a chemical synthesis of N-(4-(2-amino-3-(3-oxo-3-(piperazin-1-yl)prop-1-ynyl)pyridin-4-yloxy)-3-fluorophenyl)-1-(4-fluorophenyl)-2-oxo-1,2-dihydropyridine-3-carboxamide in an aspect of the invention.

FIG. 15 is a chemical synthesis of N-(4-(2-amino-3-(3-oxo-3-(piperazin-1-yl)prop-1-ynyl)pyridin-4-yloxy)3-fluorophenyl)-4-ethoxy-1-(4-fluorophenyl)-2-oxo-1,2-dihydropyridine-3-carboxamide in an aspect of the invention.

FIG. 16 is a chemical synthesis of N-(4-(2-amino-3-(3-oxo-3-(piperazin-1-yl)prop-1-ynyl)pyridin-4-yloxy)-3-fluorophenyl)-4-(4-fluorophenyl)-3-oxo-3,4-dihydropyrazine-2-carboxamide in an aspect of the invention.

FIG. 17 is a chemical synthesis of N-(4-(2-amino-3-(3-(4-hydroxypiperidin-1-yl)-3-oxoprop-1-ynyl)pyridin-4-yloxy)-3-fluorophenyl)-4-(4-fluorophenyl)-3-oxo-3,4-dihydropyrazine-2-carboxamide in an aspect of the invention.

FIG. 18 is a chemical synthesis of N-(4-(2-amino-3-(3-(4-(2-methoxyethyl)piperazin-1-yl)-3-oxoprop-1-ynyl)pyridin-4-yloxy)-3-fluorophenyl)-4-(4-fluorophenyl)-3-oxo-3,4-dihydropyrazine-2-carboxamide in an aspect of the invention.

FIG. 19 is a chemical synthesis of N-(4-(2-amino-3-(4-phenoxyphenyl)pyridin-4-yloxy)-3-fluorophenyl)-2-4-(fluorophenyl)-3-oxo-2,3-dihydropyridazine-4-carboxamide in an aspect of the invention.

FIG. 20 is a chemical synthesis of N-(4-(2-amino-3-(1-propyl-lh-pyrazol-4-yl)pyridin-4-yloxy)-3-fluorophenyl)-2-(4-fluorophenyl)-3-oxo-2,3-dihydropyridazine-4-carboxamide in an aspect of the invention.

FIG. 21 is a chemical synthesis of N-(4-(2-amino-3-(3-methyl-3-(piperazin-1-yl)but-1-ynyl)pyridin-4-yloxy)-3-fluorophenyl)-2-(4-fluorophenyl)-3-oxo-2,3-dihydropyridazine-4-carboxamide in an aspect of the invention.

FIG. 22 is a chemical synthesis of N-(4-(2-amino-3-(3-(4-(2-methoxyethyl)piperazin-1-yl)-3-methylbut-1-ynyl)pyridine-4-yloxy)-3-fluorophenyl)-2-(4-fluorophenyl)-3-oxo-2,3-dihydropyridazine-4-carboxamide in an aspect of the invention.

FIG. 23 is a chemical synthesis of N-(4-(2-amino-3-(3-(4-(2-methoxyethyl)piperazin-1-yl)prop-2-ynyl)pyridine-4-yloxy)-3-fluorophenyl)-3-oxo-2,3-dihydropyridazine-4-carboxamide in an aspect of the invention.

FIG. 24 is a chemical synthesis of N-(4-(2-amino-3-(piperidin-4-ylethynyl)pyridine-4-yloxy)-3-fluorophenyl)-2-(4-fluorophenyl)-3-oxo-2,3-dihydropyridazine-4-carboxamide in an aspect of the invention.

FIG. 25 is a chemical synthesis of N-(4-(2-amino-3-((1-methylpiperidin-4-yl)ethynyl)pyridin-4-yloxy)-3-fluorophenyl)-2-(4-fluorophenyl)-3-oxo-2,3-dihydropyridazine-4-carboxamide in an aspect of the invention.

FIG. 26 is a chemical synthesis of N-(4-(2-amino-3-((1-(2-methoxyethyl)piperidin-4-yl)ethynyl)pyridin-4-yloxy)-3-fluorophenyl)-2-(4-fluorophenyl)-3-oxo-2,3-dihydropyridazine-4-carboxamide in an aspect of the invention.

FIG. 27 is a chemical synthesis of N-(4-(2-amino-3-(3-methyl-3-morpholinobut-1-ynl)pyridin-4-yloxy)-3-fluorophenyl)-2-(4-fluorophenyl)-3-oxo-2,3-dihydropyridazine-4-carboxamide in an aspect of the invention.

FIG. 28 is a chemical synthesis of N-(4-(2-amino-3-(3-(4-methylpiperazin-1-yl)prop-1-ynyl)pyridin-4-yloxy)-3-fluorophenyl)-2-(4-fluorophenyl)-3-oxo-2,3-dihydropyridazine-4-carboxamide in an aspect of the invention.

FIG. 29 is a chemical synthesis of N-(4-(2-amino-3-(3-(piperidin-4-yl)prop-1-ynyl)pyridin-4-yloxy)-3-fluorophenyl)-2-(4-fluorophenyl)-3-oxo-2,3-dihydropyridazine-4-carboxamide in an aspect of the invention.

FIG. 30 is a chemical synthesis of N-(4-(2-amino-3-(3-(1-(2-methoxyethyl)piperidin-4-yl)prop-1-ynyl)pyridin-4-yloxy)-3-fluorophenyl)-2-(4-fluorophenyl)-3-oxo-2,3-dihydropyridazine-4-carboxamide in an aspect of the invention.

FIG. 31 is a chemical synthesis of N-(4-(2-amino-3-(3-(1-isopropylpiperidin-4-yl)prop-1-ynyl)pyridin-4-yloxy)-3-fluorophenyl)-2-(4-fluorophenyl)-3-oxo-2,3-dihydropyridazine-4-carboxamide in an aspect of the invention.

### DETAILED DESCRIPTION OF THE INVENTION

A compound of Formula (I) or a pharmaceutically acceptable salt thereof:

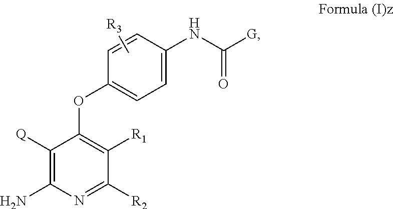

wherein:

R¹ is H, alkyl, haloalkyl, halo, or CN;

R² is H, alkyl, haloalkyl, halo, or CN;

R³ is H or halo;

Q is H, CN, halo, alkyl, alkenyl, alkynyl, cycloalkyl, heterocyclyl, heteroaryl, or aryl, wherein said alkenyl or alkynyl is selected from the group consisting of

—CH═CR⁴(CX′)ₘ(CH₂)ₙNR⁵R⁶, —C≡C(CX′)ₘ(CH₂)ₙNR⁵R⁶,

—CH═CR⁴(CX′)ₘ(CH₂)ₙCHR⁵R⁶, —C≡C(CX′)ₘ(CH₂)ₙCHR⁵R⁶,

—CH═CR⁴(CX′)ₘ(CH₂)ₙNR⁷OR⁸, and —C≡C(CX′)ₘ(CH₂)ₙNR⁷OR⁸;

wherein

R⁴ is hydrogen or halo;

X′ is H₂, (C₁₋₆ alkyl)₂, or ═O;

m is 0 or 1;

n is 0 or 1-3;

—NR⁵R⁶ either forms a 4-7 membered heterocyclic ring or does not form a ring structure, the heterocyclic ring being either heteroaryl or heterocyclyl ring,

when —NR⁵R⁶ forms a 4-7 membered heterocyclic ring, the 4-7 membered heterocyclic ring includes an optional second heteroatom in addition to the nitrogen of —NR⁵R⁶ and is optionally substituted with one or more substituent groups independently selected from the group consisting of linear C₁-C₆ alkyl, branched C₃-C₆ alkyl, hydroxy, C₁-C₆ alkoxyalkyl, carboxylic acid, linear C₁-C₄ alkyl carboxylic acid, and branched C₃-C₄ alkyl carboxylic acid;

when —NR⁵R⁶ does not form a ring structure, R⁵ is selected from the group consisting of hydrogen, linear C₁-C₆ alkyl, and branched C₃-C₆ alkyl, and R⁶ is selected from the group consisting of hydrogen, linear C₁-C₆ alkyl optionally substituted with at least one fluoro or at least one hydroxy, branched C₃-C₆ alkyl optionally substituted with at least one fluoro or at least one hydroxy, and cycloalkyl optionally substituted with at least one fluoro or at least one hydroxy;

—CHR⁵R⁶ either forms a 4-7 membered heterocyclic ring or does not form a ring structure, the heterocyclic ring being either heteroaryl or heterocyclyl ring,

when —CHR⁵R⁶ forms a 4-7 membered heterocyclic ring, the 4-7 membered heterocyclic ring includes one or two heteroatoms and is optionally substituted with one or more substituent groups independently selected from the group consisting of linear C₁-C₆ alkyl, branched C₃-C₆ alkyl, hydroxy, C₁-C₆ alkoxyalkyl, carboxylic acid, linear C₁-C₄ alkyl carboxylic acid, and branched C₃-C₄ alkyl carboxylic acid;

when —CHR⁵R⁶ does not form a ring structure, R⁵ is selected from the group consisting of hydrogen, linear C₁-C₆ alkyl, and branched C₃-C₆ alkyl, and R⁶ is selected from the group consisting of hydrogen, linear C₁-C₆ alkyl optionally substituted with at least one fluoro or at least one hydroxy, branched C₃-C₆ alkyl optionally substituted with at least one fluoro or at least one hydroxy, and cycloalkyl optionally substituted with at least one fluoro or at least one hydroxy;

—NR⁷OR⁸ does not form a ring structure, R⁷ is selected from the group consisting of hydrogen, linear C₁-C₆ alkyl, and branched C₃-C₆ alkyl, and R⁸ is selected from the group consisting of hydrogen, linear C₁-C₆ alkyl optionally substituted with at least one fluoro, hydroxy, or alkoxy group, branched C₃-C₆ alkyl optionally substituted with at least one fluoro, hydroxy, or alkoxy group, and cycloalkyl optionally substituted with at least one fluoro, hydroxy, or alkoxy group;

G is

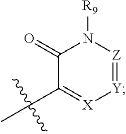

wherein

R⁹ is phenyl substituted with alkyl, haloalkyl, halo, and/or CN;

each X, Y, and Z is independently CR¹⁰ or N; and

R¹⁰ is H, C₁-C₆ alkyl, or C₁-C₆ alkoxy.

The pyridazinone-based inhibitors are useful in treating a variety of diseases and disorders associated with AXL, Mer, and/or c-Met without the need for specialized mode of administration.

In some aspects of Formula (I), both R¹ and R² are hydrogen.

In some aspects of Formula (I), R³ is a halo.

In some aspects of Formula (I), Q is CN, halo, optionally substituted phenyl, optionally substituted heterocyclyl, or an alkenyl or alkynyl moiety selected from the group consisting of —CH═CR⁴(CX′)ₘ(CH₂)ₙNR⁵R⁶, —C≡C(CX′)ₘ(CH₂)ₙNR⁵R⁶, —CH═CR⁴(CX′)ₘ(CH₂)ₙCHR⁵R⁶, —C≡C(CX′)ₘ(CH₂)ₙCHR⁵R⁶, —CH═CR⁴(CX′)ₘ(CH₂)ₙNR⁷OR⁸, and —C≡C(CX′)ₘ(CH₂)ₙNR⁷OR⁸,

wherein

R⁴ is hydrogen or halo;

X′ is H₂, (C₁₋₆ alkyl)₂, or ═O;

m is 0 or 1;

n is 0 or 1;

—NR⁵R⁶ is morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl,

—CHR⁵R⁶ is tetrahydropyranyl, morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl,

R⁷ is selected from the group consisting of hydrogen, linear C₁-C₆ alkyl, and branched C₃-C₆ alkyl, and

R⁸ is selected from the group consisting of linear C₁-C₆ alkyl optionally substituted with at least one alkoxy group and branched C₃-C₆ alkyl optionally substituted with at least one alkoxy group.

In some aspects of Formula (I), R⁹ is phenyl substituted with alkyl, haloalkyl, halo, and/or CN; and either (i) X is N, and Y and Z are CH, (ii) X and Y are CH, and Z is N, or (iii) X, Y, and Z are each CR¹⁰.

In some aspects, the compound of Formula (I) is a compound Formula (Ib):

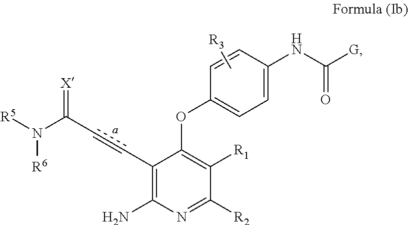

wherein [symbol] is —C≡C— or —CH═CH—.

In some aspects of Formula (Ib), both R¹ and R² are hydrogen.

In some aspects of Formula (Ib), R³ is a halo.

In some aspects of Formula (Ib), X′ is H₂, (C₁₋₆ alkyl)₂, or ═O; and —NR⁵R⁶ is morpholinyl, piperazinyl, or piperidinyl, each of which is optionally substituted with one or more substituent groups independently selected from the group consisting of a nitrogen protecting group (e.g., tert-butyloxycarbonyl (Boc), fluorenylmethyloxycarbonyl (Fmoc), carboxybenzyl (Cbz), acetyl, trifluoroacetamide, phthalimide, benzyl, trityl, benzylideneamine, or tosyl), alkyl, hydroxy, alkoxy, and alkoxyalkyl.

In some aspects of Formula (Ib), R⁹ is phenyl substituted with alkyl, haloalkyl, halo, and/or CN; and either (i) X is N, and Y and Z are CH, (ii) X and Y are CH, and Z is N, or (iii) X, Y, and Z are each CR¹⁰.

In some aspects, the compound of Formula (I) is a compound Formula (Ic):

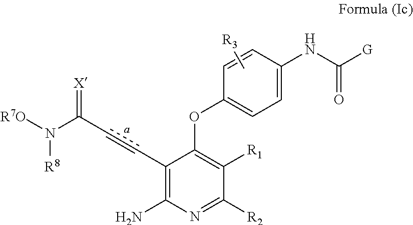

wherein [symbol] is —C≡C— or —CH═CH—.

In some aspects of Formula (Ic), both R¹ and R² are hydrogen.

In some aspects of Formula (Ic), R³ is a halo.

In some aspects of Formula (Ic), X′ is H₂, (C₁₋₆ alkyl)₂, or ═O; R⁷ is selected from the group consisting of linear C₁-C₆ alkyl and branched C₃-C₆ alkyl; and R⁸ is selected from the group consisting of linear C₁-C₆ alkyl and branched C₃-C₆ alkyl.

In some aspects of Formula (Ic), R⁹ is phenyl substituted with alkyl, haloalkyl, halo, and/or CN; and either (i) X is N, and Y and Z are CH, (ii) X and Y are CH, and Z is N, or (iii) X, Y, and Z are each CR¹⁰.

Exemplary compounds of Formula (I), including compounds of Formulas (Ib) and (Ic), are set forth below in the examples. Pharmaceutically acceptable salts of these exemplary compounds are also envisioned. In particular, the compound of Formula (I) is selected from

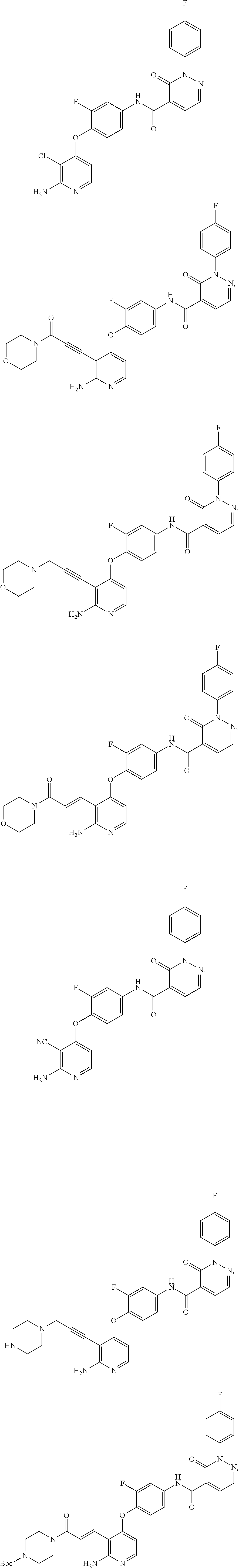

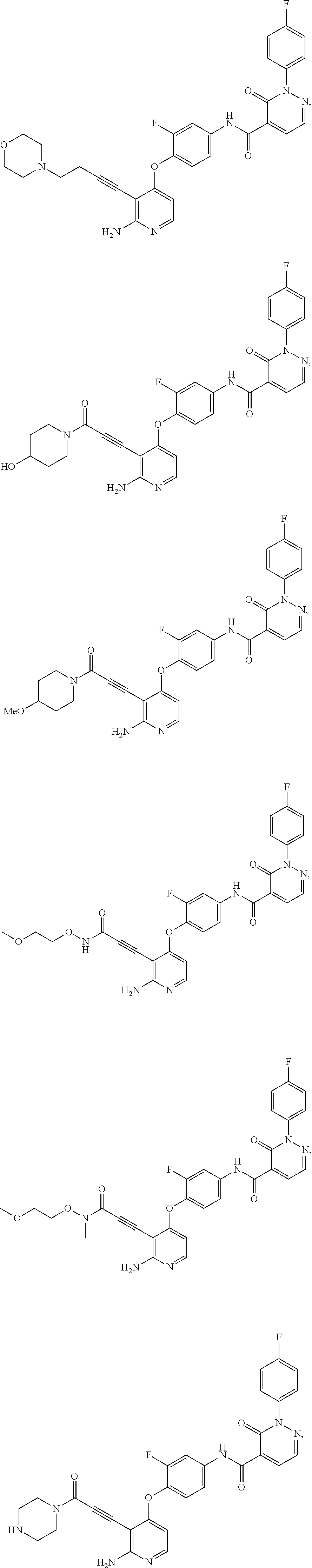 [SMILES: Nc1nccc(Oc2ccc(NC(=O)c3ccnn(-c4ccc(F)cc4)c3=O)cc2F)c1C#CCCN1CCOCC1 ; Nc1nccc(Oc2ccc(NC(=O)c3ccnn(-c4ccc(F)cc4)c3=O)cc2F)c1C#CC(=O)N1CCC(O)CC1 ; COC1CCN(C(=O)C#Cc2c(Oc3ccc(NC(=O)c4ccnn(-c5ccc(F)cc5)c4=O)cc3F)ccnc2N)CC1 ; COCCONC(=O)C#Cc1c(Oc2ccc(NC(=O)c3ccnn(-c4ccc(F)cc4)c3=O)cc2F)ccnc1N ; COCCON(C)C(=O)C#Cc1c(Oc2ccc(NC(=O)c3ccnn(-c4ccc(F)cc4)c3=O)cc2F)ccnc1N ; Nc1nccc(Oc2ccc(NC(=O)c3ccnn(-c4ccc(F)cc4)c3=O)cc2F)c1C#CC(=O)N1CCNCC1]

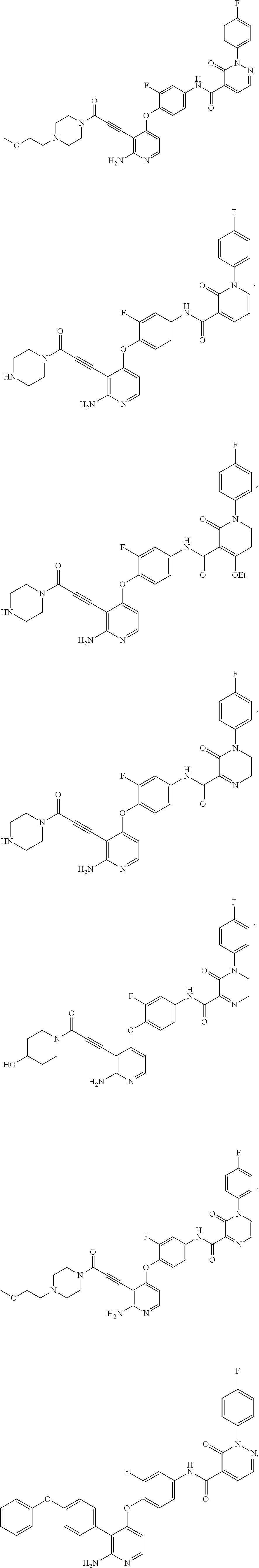

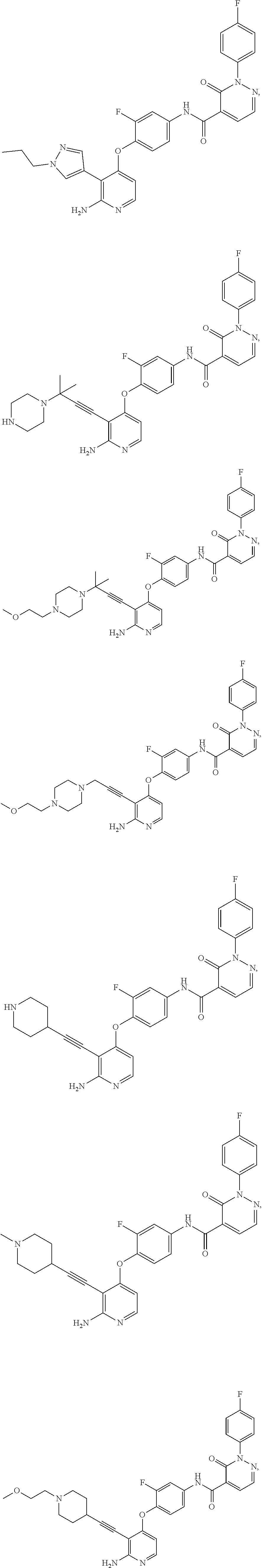

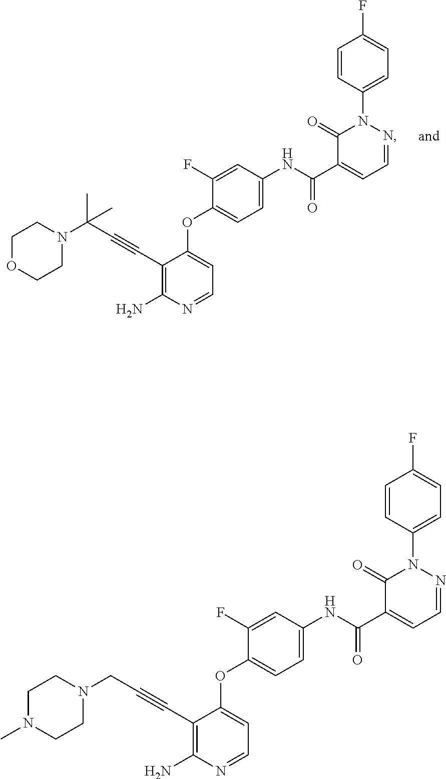 [SMILES: CC(C)(C#Cc1c(Oc2ccc(NC(=O)c3ccnn(-c4ccc(F)cc4)c3=O)cc2F)ccnc1N)N1CCOCC1 ; "and" ; CN1CCN(CC#Cc2c(Oc3ccc(NC(=O)c4ccnn(-c5ccc(F)cc5)c4=O)cc3F)ccnc2N)CC1]

or a pharmaceutically acceptable salt thereof.

In any of the aspects above, the term “alkyl” implies a straight-chain or branched alkyl substituent containing from, for example, from about 1 to about 8 carbon atoms, e.g., from about 1 to about 6 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in C₃-C₆ cycloalkylalkyl, hydroxyalkyl, haloalkyl (e.g., monohaloalkyl, dihaloalkyl, and trihaloalkyl), cyanoalkyl, aminoalkyl, alkylamino, dialkylamino, arylalkyl, etc. The alkyl can be substituted or unsubstituted, as described herein. Even in instances in which the alkyl is an alkylene chain (e.g., —(CH₂)ₙ—), the alkyl group can be substituted or unsubstituted. An example of a substituted alkylene chain includes —CH₂CH₂-methoxy.

In any of the aspects above, the term “alkenyl,” as used herein, means a linear alkenyl substituent containing from, for example, about 2 to about 8 carbon atoms (branched alkenyls are about 3 to about 8 carbons atoms), e.g., from about 3 to about 6 carbon atoms (branched alkenyls are about 3 to about 6 carbons atoms). In accordance with an aspect, the alkenyl group is a C₂-C₄ alkenyl. Examples of alkenyl group include ethenyl, allyl, 2-propenyl, 1-butenyl, 2-butenyl, 1-pentenyl, 2-pentenyl, 3-pentenyl, 1-hexenyl, and the like. The alkenyl can be substituted or unsubstituted, as described herein.

In any of the aspects above, the term “alkynyl,” as used herein, means a linear alkynyl substituent containing at least one carbon-carbon triple bond and from, for example, about 2 to about 8 carbon atoms (branched alkynyls are about 4 to about 12 carbons atoms), e.g., from about 2 to about 6 carbon atoms (branched alkynyls can be from about 4 to about 8 carbon atoms), e.g., from about 2 to about 4 carbon atoms. Examples of such substituents include propynyl, propargyl, n-butynyl, pentynyl, isopentynyl, hexynyl, octynyl, and the like. The alkynyl can be substituted or unsubstituted, as described herein.

In any of the aspects above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl can be substituted or unsubstituted, as described herein.

In any of the aspects above, the term “hydroxy” refers to the group —OH.

In any of the aspects above, the terms “alkoxy” embrace linear or branched alkyl groups that are attached to a divalent oxygen. The alkyl group is the same as described herein.

In any of the aspects above, the term “halo” refers to a halogen radical selected from fluoro, chloro bromo, and iodo.

In any of the aspects above, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2π electrons, according to Hückel's Rule, wherein n=1, 2, or 3. This definition also applies wherever “aryl” occurs as part of a group, such as, e.g., in haloaryl (e.g., monohaloaryl, dihaloaryl, and trihaloaryl), arylalkyl, etc. The aryl can be substituted or unsubstituted, as described herein.

In any of the aspects above, the term “heteroaryl” refers to aromatic 5 or 6 membered monocyclic groups, 9 or 10 membered bicyclic groups, and 11 to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)- and (1,2,4)-triazolyl, pyrazinyl, tetrazolyl, furyl, pyrrolyl, thienyl, isothiazolyl, thiazolyl, isoxazolyl, and oxadiazolyl. The heteroaryl can be substituted or unsubstituted, as described herein.

The term “heterocyclyl” means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocyclyl is a 5, 6, or 7-membered monocyclic ring and contains one, two, or three heteroatoms selected from nitrogen, oxygen, and sulfur. The heterocyclyl may be attached to the parent structure through a carbon atom or through any heteroatom of the heterocyclyl that results in a stable structure (e.g., a nitrogen atom). Examples of such heterocyclyl rings are isoxazolyl, thiazolinyl, imidazolidinyl, piperazinyl, homopiperazinyl, pyrrolyl, pyrrolinyl, pyrazolyl, pyranyl, dihydropyranyl, tetraydropyranyl, piperidinyl, oxazolyl, and morpholinyl. Preferably, the heterocyclyl is piperazinyl, piperidinyl, or morpholinyl. The heterocyclyl can be substituted or unsubstituted, as described herein.

In other aspects, any substituent that is not hydrogen (e.g., alkyl, alkenyl, alkynyl, cycloalkyl, aryl, arylalkyl, heteroaryl, heteroarylalkyl, or heterocyclyl) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present invention. Suitable substituents include, e.g., halo, alkyl, alkenyl, alkynyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocyclyl, each of which is described herein.

In any of the aspects above, whenever a range of the number of atoms in a structure is indicated (e.g., a C₁₋₁₂, C₁₋₈, C₁₋₆, or C₁₋₄ alkyl, cycloalkyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

The subscript “m” represents the number of (CX′) repeat units. The subscript m can be either 0 or 1. When m is 0, then (CX′) is not present in the molecule.

The subscript “n” represents the number of methylene (CH₂) repeat units. The subscript n can be either 0 or an integer from 1-3 (i.e., 1, 2, or 3). When n is 0, then the respective moiety does not contain any methylene repeat units.

In any of the aspects herein, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977). For example, the salt can be selected from the group consisting of acetate, benzoate, besylate, bitartrate, bromide, carbonate, chloride, edetate, edisylate, estolate, fumarate, gluceptate, gluconate, hydrobromide, hydrochloride, iodide, formate, lactate, lactobionate, malate, maleate, mandelate, mesylate, methyl bromide, methyl sulfate, mucate, napsylate, nitrate, oxalate, pamoate, phosphate, diphosphate, salicylate, disalicylate, stearate, succinate, sulfate, tartrate, tosylate, triethiodide, trifluoroacetate, and valerate.

The methods described herein comprise administering a compound of Formula (I) or a pharmaceutically acceptable salt thereof in the form of a pharmaceutical composition. In particular, a pharmaceutical composition will comprise at least one compound of Formula (I) or a pharmaceutically acceptable salt thereof and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the pharmaceutical composition is administered orally or intravenously.

In accordance with any of the aspects, the compound of Formula (I) or a pharmaceutically acceptable salt thereof can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of Formula (I) or a salt thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (e) mixtures thereof.

The parenteral formulations will typically contain from about 0.5 to about 25% by weight of the inhibitors in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The inhibitors can be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution, such as a mouthwash. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The compound of Formula (I) or a pharmaceutically acceptable salt thereof, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The dose administered to the subject, particularly a human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular inhibitor and the desired effect. It will be appreciated by one of skill in the art that various conditions or disease states may require prolonged treatment involving multiple administrations.

The inventive methods comprise administering an effective amount of a compound of Formula (I) or a pharmaceutically acceptable salt thereof. An “effective amount” means an amount sufficient to show a meaningful benefit in an individual, cell, or tissue to be great. A meaningful benefit means that one or more symptoms of the disease or disorder (e.g., asthma, cancer) are prevented, reduced, halted, or eliminated subsequent to administration of a compound of Formula (I), including a compound of Formula (Ib) or (Ic), or a pharmaceutically acceptable salt thereof, thereby effectively treating the disease to at least some degree. For example, the meaningful benefit can be promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular disease or disorder. The meaningful benefit observed in the subject to be treated can be to any suitable degree (10, 20, 30, 40, 50, 60, 70, 80, 90% or more).

Effective amounts may vary depending upon the biological effect desired in the individual, condition to be treated, and/or the specific characteristics of the compound of Formula (I), including a compound of Formula (Ib) or (Ic), or a pharmaceutically acceptable salt thereof, and the individual. In this respect, any suitable dose of the compound of Formula (I) or a pharmaceutically acceptable salt thereof can be administered to the subject (e.g., human), according to the disease or disorder (e.g., asthma, cancer) to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound of Formula (I), including a compound of Formula (Ib) or (Ic), or a pharmaceutically acceptable salt thereof desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

In an aspect, a compound of Formula (I) or a salt thereof inhibits one or more enzymes selected from AXL, Mer, and c-Met. Accordingly, the present invention provides a method of inhibiting an AXL, Mer, and/or c-Met enzyme in a cell comprising administering a pharmaceutically effective amount of a compound of Formula (I) or a pharmaceutically acceptable salt thereof to a cell in need of such inhibition (e.g., a cell that overexpresses AXL, Mer, and/or c-Met). For example, the cell can be any cell that overexpresses AXL, Mer, and/or c-Met and is associated with any suitable tissue, particularly a tissue associated with a disease, such as from papillary thyroid carcinoma, pancreatic cancer, lung cancer, colon cancer, breast carcinoma, neuroblastoma, pain, cachexia (wasting syndrome), dermatitis, and asthma. The tissue can be from, for example, the thyroid, pancreas, lung, colon, breast, skin, or adrenal glands. In accordance with an aspect, the cell is a cancer cell that overexpresses AXL, Mer, and/or c-Met, such as cells from papillary thyroid carcinoma, pancreatic cancer, lung cancer, colon cancer, breast carcinoma, and neuroblastoma. In another aspect, the cancer cells are non-small cell lung cancer cells.

Elevated levels of AXL, Mer, and c-Met are associated with certain diseases, and it is envisioned that inhibiting one or more of AXL, Mer, and c-Met is a viable treatment of such diseases. Thus, the invention provides a method of treating or preventing an AXL-, Mer- and/or c-Met-mediated disease in a subject with a compound of Formula (I). In general, the compound of Formula (I) will be provided to the subject in the form of a pharmaceutical composition, as described herein. The type of disease to be treated or prevented is not particularly limited, but in general, the disease is characterized as having increased expression of AXL, Mer, and c-Met relative to normal tissue of the same type. In some aspects, the disease is selected from the group consisting of papillary thyroid carcinoma, pancreatic cancer, lung cancer, colon cancer, breast carcinoma, neuroblastoma, pain, cachexia (wasting syndrome), dermatitis, and asthma. The method comprises administering a pharmaceutically effective amount of a compound of Formula (I) or a pharmaceutically acceptable salt thereof to a subject in need of such treatment. In some preferred aspects of this method, the disease is lung cancer (e.g., non-small cell lung cancer).

The invention further provides a method of treating a subject with cancer cells resistant to an anti-cancer agent, comprising administering to the subject an effective amount of the compound of Formula (I), including a compound of Formula (Ib) or (Ic), or a pharmaceutically acceptable salt thereof, and the anti-cancer agent, whereby the compound or pharmaceutically acceptable salt thereof re-sensitizes the cancer cells to the anti-cancer agent. The cancer cell is the same as described herein. In accordance with an aspect, the cancer cells are selected from papillary thyroid carcinoma, pancreatic cancer, lung cancer, colon cancer, breast carcinoma, and neuroblastoma. In another aspect, the cancer cells are non-small cell lung cancer cells.

In certain aspects of this method, the compound of Formula (I), including a compound of Formula (Ib) or (Ic), or a pharmaceutically acceptable salt thereof can be co-administered with an anti-cancer agent (e.g., a chemotherapeutic agent) and/or radiation therapy. In an aspect, the method comprises administering an amount of a compound or salt thereof, preferably in the form of a pharmaceutical composition, that is effective to sensitize the cancer cells to one or more therapeutic regimens (e.g., chemotherapy or radiation therapy). The terms “co-administered” or “co-administration” refer to simultaneous or sequential administration. A compound can be administered before, concurrently with, or after administration of another compound using any suitable time frame.

One or more than one, e.g., two, three, or more anti-cancer agents can be administered. In this regard, the present invention is directed a pharmaceutical composition comprising a pharmaceutically acceptable carrier and a combination of the compound of Formula (I), including a compound of Formula (Ib) or (Ic), or a pharmaceutically acceptable salt thereof and at least one anti-cancer agent (e.g., chemotherapeutic agent).

Examples of anti-cancer agents include platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mitomycin C, plicamycin, dactinomycin), taxanes (e.g., paclitaxel and docetaxel), antimetabolites (e.g., 5-fluorouracil, cytarabine, pemetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panitumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), a CDK4/6 inhibitor (e.g., abemaciclib, palbociclib, ribociclib), anti-cancer hormonal agents (e.g., tamoxifen, fulvestrant, raloxifene, leuprolide, bicalutamide, granisetron, flutamide, goserelin), aromatase inhibitors (e.g., exemestane, letrozole, and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, immune checkpoint inhibitors (e.g., anti-PD1, anti-CTLA4, and anti-PD-L1), cellular immunotherapy (e.g., chimeric antigen receptor T cell therapy, tumor-infiltrating lymphocyte therapy), or any combination thereof.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

The invention is further illustrated by the following aspects.

A compound of Formula (I) or a pharmaceutically acceptable salt thereof:

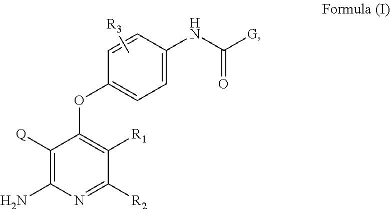

wherein:

R¹ is H, alkyl, haloalkyl, halo, or CN;

R² is H, alkyl, haloalkyl, halo, or CN;

R³ is H or halo;

Q is H, CN, halo, alkyl, alkenyl, alkynyl, cycloalkyl, heterocyclyl, heteroaryl, or aryl, wherein said alkenyl or alkynyl is selected from the group consisting of

—CH═CR⁴(CX′)ₘ(CH₂)ₙNR⁵R⁶, —C≡C(CX′)ₘ(CH₂)ₙNR⁵R⁶,

—CH═CR⁴(CX′)ₘ(CH₂)ₙCHR⁵R⁶, —C≡C(CX′)ₘ(CH₂)ₙCHR⁵R⁶,

—CH═CR⁴(CX′)ₘ(CH₂)ₙNR⁷OR⁸, and —C≡C(CX′)ₘ(CH₂)ₙNR⁷OR⁸;

wherein

R⁴ is hydrogen or halo;

X′ is H₂, (C₁₋₆ alkyl)₂, or ═O;

m is 0 or 1;

n is 0 or 1-3;

—NR⁵R⁶ either forms a 4-7 membered heterocyclic ring or does not form a ring structure, the heterocyclic ring being either heteroaryl or heterocyclyl ring,

when —NR⁵R⁶ forms a 4-7 membered heterocyclic ring, the 4-7 membered heterocyclic ring includes an optional second heteroatom in addition to the nitrogen of —NR⁵R⁶ and is optionally substituted with one or more substituent groups independently selected from the group consisting of linear C₁-C₆ alkyl, branched C₃-C₆ alkyl, hydroxy, C₁-C₆ alkoxyalkyl, carboxylic acid, linear C₁-C₄ alkyl carboxylic acid, and branched C₃-C₄ alkyl carboxylic acid;

when —NR⁵R⁶ does not form a ring structure, R⁵ is selected from the group consisting of hydrogen, linear C₁-C₆ alkyl, and branched C₃-C₆ alkyl, and R⁶ is selected from the group consisting of hydrogen, linear C₁-C₆ alkyl optionally substituted with at least one fluoro or at least one hydroxy, branched C₃-C₆ alkyl optionally substituted with at least one fluoro or at least one hydroxy, and cycloalkyl optionally substituted with at least one fluoro or at least one hydroxy;

—CHR⁵R⁶ either forms a 4-7 membered heterocyclic ring or does not form a ring structure, the heterocyclic ring being either heteroaryl or heterocyclyl ring,

when —CHR⁵R⁶ forms a 4-7 membered heterocyclic ring, the 4-7 membered heterocyclic ring includes one or two heteroatoms and is optionally substituted with one or more substituent groups independently selected from the group consisting of linear C₁-C₆ alkyl, branched C₃-C₆ alkyl, hydroxy, C₁-C₆ alkoxyalkyl, carboxylic acid, linear C₁-C₄ alkyl carboxylic acid, and branched C₃-C₄ alkyl carboxylic acid;

when —CHR⁵R⁶ does not form a ring structure, R⁵ is selected from the group consisting of hydrogen, linear C₁-C₆ alkyl, and branched C₃-C₆ alkyl, and R⁶ is selected from the group consisting of hydrogen, linear C₁-C₆ alkyl optionally substituted with at least one fluoro or at least one hydroxy, branched C₃-C₆ alkyl optionally substituted with at least one fluoro or at least one hydroxy, and cycloalkyl optionally substituted with at least one fluoro or at least one hydroxy;

—NR⁷OR⁸ does not form a ring structure, R⁷ is selected from the group consisting of hydrogen, linear C₁-C₆ alkyl, and branched C₃-C₆ alkyl, and R⁸ is selected from the group consisting of hydrogen, linear C₁-C₆ alkyl optionally substituted with at least one fluoro, hydroxy, or alkoxy group, branched C₃-C₆ alkyl optionally substituted with at least one fluoro, hydroxy, or alkoxy group, and cycloalkyl optionally substituted with at least one fluoro, hydroxy, or alkoxy group;

### G is

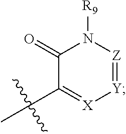

wherein

R⁹ is phenyl substituted with alkyl, haloalkyl, halo, and/or CN;

each X, Y, and Z is independently CR¹⁰ or N; and

R¹⁰ is H, C₁-C₆ alkyl, or C₁-C₆ alkoxy.

2. The compound of aspect 1 or a pharmaceutically acceptable salt thereof, wherein both R¹ and R² are hydrogen.

3. The compound of aspect 1 or 2 or a pharmaceutically acceptable salt thereof, wherein R³ is a halo.

4. The compound of any one of aspects 1-3 or a pharmaceutically acceptable salt thereof, wherein Q is CN, halo, optionally substituted phenyl, optionally substituted heterocyclyl, or an alkenyl or alkynyl moiety selected from the group consisting of —CH═CR⁴(CX′)ₘ(CH₂)ₙNR⁵R⁶, —C≡C(CX′)ₘ(CH₂)ₙNR⁵R⁶, —CH═CR⁴(CX′)ₘ(CH₂)ₙCHR⁵R⁶, —C≡C(CX′)ₘ(CH₂)ₙCHR⁵R⁶, —CH═CR⁴(CX′)ₘ(CH₂)ₙNR⁷OR⁸, and —C≡C(CX′)ₘ(CH₂)ₙNR⁷OR⁸, wherein R⁴ is hydrogen or halo; X′ is H₂, (C₁₋₆ alkyl)₂, or ═O; m is 0 or 1; n is 0 or 1; —NR⁵R⁶ is morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl, —CHR⁵R⁶ is tetrahydropyranyl, morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl, R⁷ is selected from the group consisting of hydrogen, linear C₁-C₆ alkyl, and branched C₃-C₆ alkyl, and R⁸ is selected from the group consisting of linear C₁-C₆ alkyl optionally substituted with at least one alkoxy group and branched C₃-C₆ alkyl optionally substituted with at least one alkoxy group.

5. The compound of any one of aspects 1-4 or a pharmaceutically acceptable salt thereof, wherein R⁹ is phenyl substituted with alkyl, haloalkyl, halo, and/or CN; and either (i) X is N, and Y and Z are CH, (ii) X and Y are CH, and Z is N, or (iii) X, Y, and Z are each CR¹⁰.

6. The compound of any one of aspects 1-5 or a pharmaceutically acceptable salt thereof, wherein the compound of Formula (I) is a compound Formula (Ib):

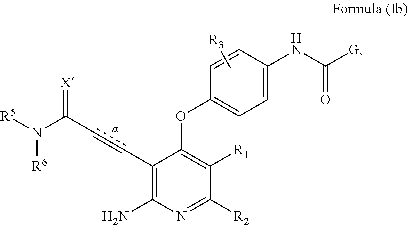

wherein [symbol] is —C≡C— or —CH═CH—.

7. The compound of aspect 6 or a pharmaceutically acceptable salt thereof, wherein both R¹ and R² are hydrogen.

8. The compound of aspect 6 or 7 or a pharmaceutically acceptable salt thereof, wherein R³ is a halo.

9. The compound of any one of aspects 6-8 or a pharmaceutically acceptable salt thereof, wherein X′ is H₂, (C₁₋₆ alkyl)₂, or ═O; and —NR⁵R⁶ is morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl.

10. The compound of any one of aspects 6-9 or a pharmaceutically acceptable salt thereof, wherein R⁹ is phenyl substituted with alkyl, haloalkyl, halo, and/or CN; and either (i) X is N, and Y and Z are CH, (ii) X and Y are CH, and Z is N, or (iii) X, Y, and Z are each CR¹⁰.

11. The compound of any one of aspects 1-5 or a pharmaceutically acceptable salt thereof, wherein the compound of Formula (I) is a compound Formula (Ic):

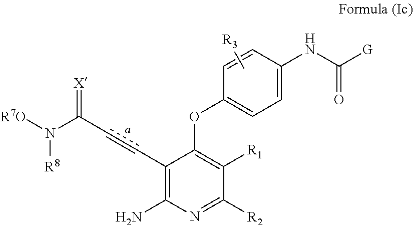

wherein [symbol] is —C≡C— or —CH═CH—.

12. The compound of aspect 11 or a pharmaceutically acceptable salt thereof, wherein both R¹ and R² are hydrogen.

13. The compound of aspect 11 or 12 or a pharmaceutically acceptable salt thereof, wherein R³ is a halo.

14. The compound of any one of aspects 11-13 or a pharmaceutically acceptable salt thereof, wherein X′ is H₂, (C₁₋₆ alkyl)₂, or ═O; R⁷ is selected from the group consisting of linear C₁-C₆ alkyl and branched C₃-C₆ alkyl; and R⁸ is selected from the group consisting of linear C₁-C₆ alkyl and branched C₃-C₆ alkyl.

15. The compound of any one of aspects 11-14 or a pharmaceutically acceptable salt thereof, wherein R⁹ is phenyl substituted with alkyl, haloalkyl, halo, and/or CN; and either (i) X is N, and Y and Z are CH, (ii) X and Y are CH, and Z is N, or (iii) X, Y, and Z are each CR¹⁰.

16. A compound of aspect 1 selected from

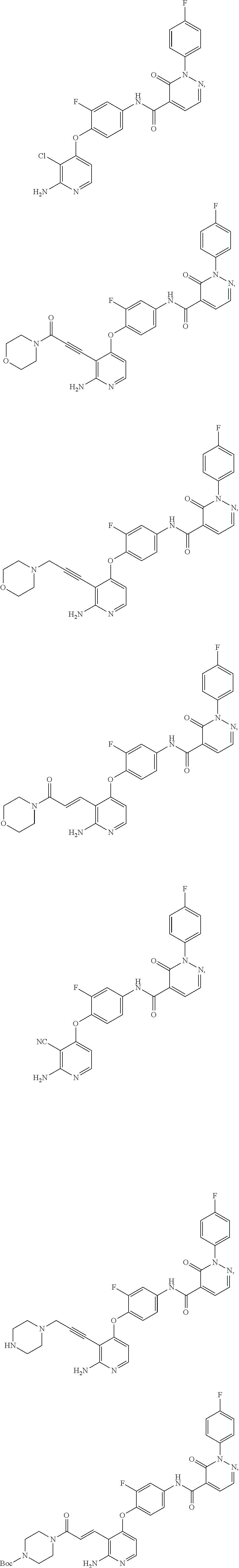

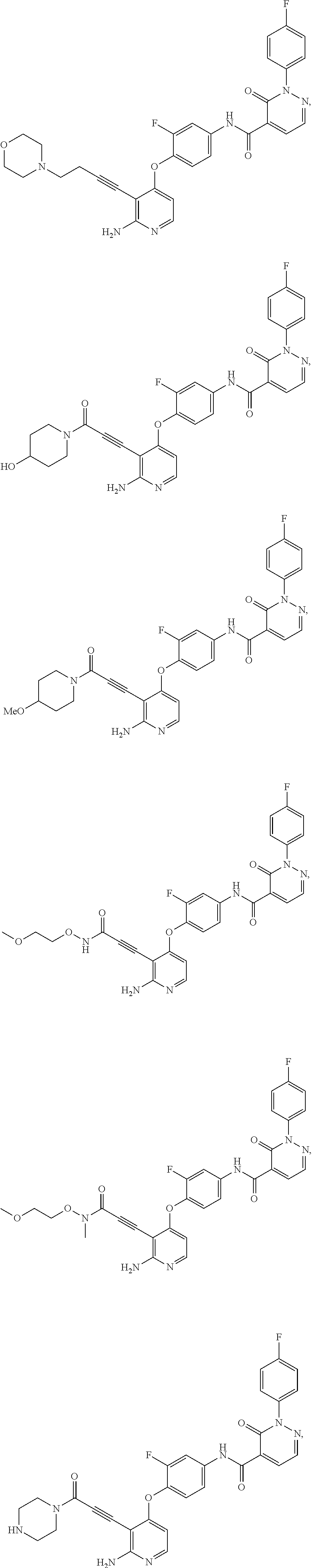 [SMILES: Nc1nccc(Oc2ccc(NC(=O)c3ccnn(-c4ccc(F)cc4)c3=O)cc2F)c1C#CCCN1CCOCC1 ; Nc1nccc(Oc2ccc(NC(=O)c3ccnn(-c4ccc(F)cc4)c3=O)cc2F)c1C#CC(=O)N1CCC(O)CC1 ; COC1CCN(C(=O)C#Cc2c(Oc3ccc(NC(=O)c4ccnn(-c5ccc(F)cc5)c4=O)cc3F)ccnc2N)CC1 ; COCCONC(=O)C#Cc1c(Oc2ccc(NC(=O)c3ccnn(-c4ccc(F)cc4)c3=O)cc2F)ccnc1N ; COCCON(C)C(=O)C#Cc1c(Oc2ccc(NC(=O)c3ccnn(-c4ccc(F)cc4)c3=O)cc2F)ccnc1N ; Nc1nccc(Oc2ccc(NC(=O)c3ccnn(-c4ccc(F)cc4)c3=O)cc2F)c1C#CC(=O)N1CCNCC1]

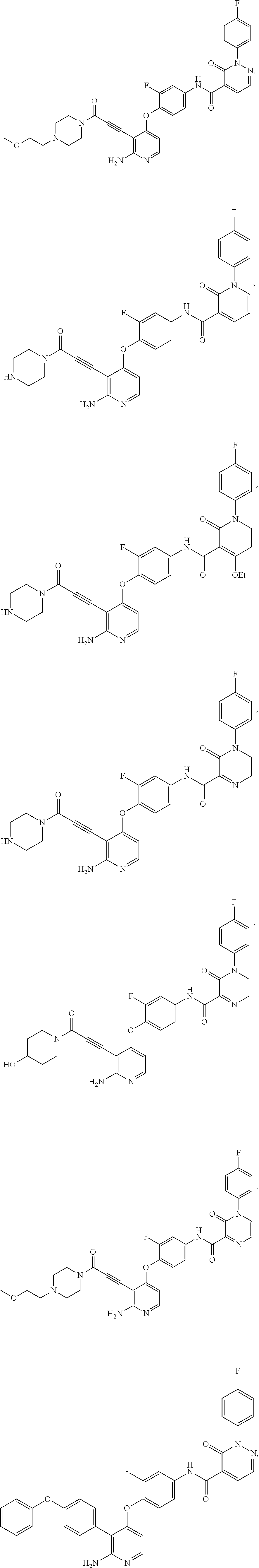

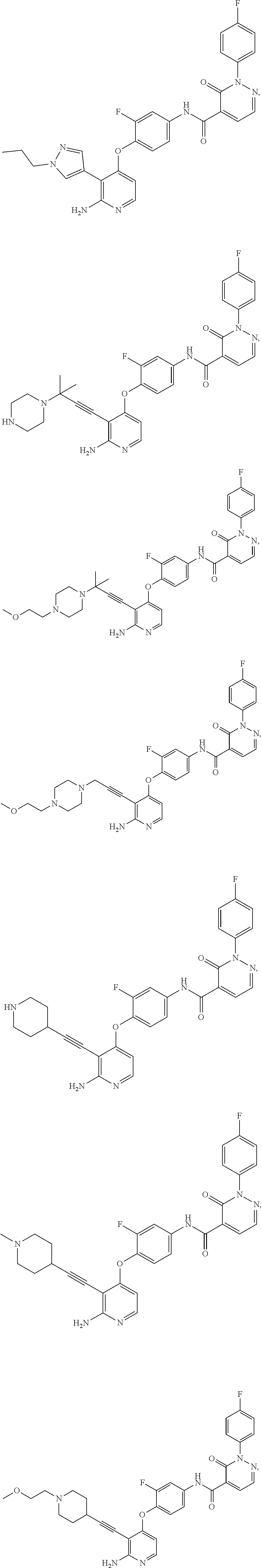

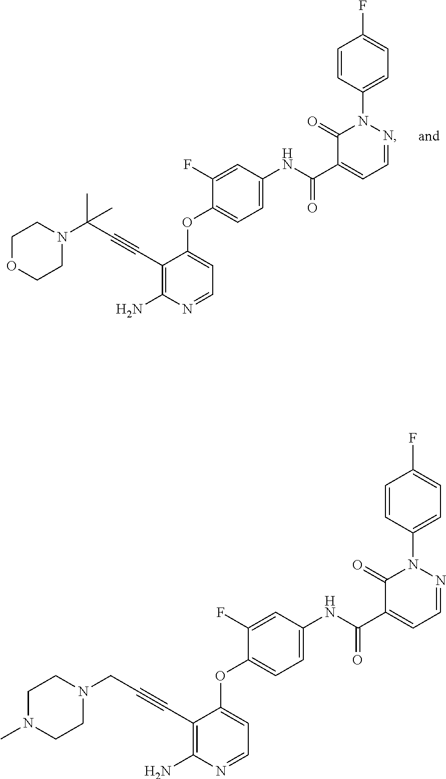 [SMILES: CC(C)(C#Cc1c(Oc2ccc(NC(=O)c3ccnn(-c4ccc(F)cc4)c3=O)cc2F)ccnc1N)N1CCOCC1 ; "and" ; CN1CCN(CC#Cc2c(Oc3ccc(NC(=O)c4ccnn(-c5ccc(F)cc5)c4=O)cc3F)ccnc2N)CC1]

or a pharmaceutically acceptable salt thereof.

17. A pharmaceutical composition comprising at least one compound of any one of aspects 1-16 or a pharmaceutically acceptable salt thereof and a pharmaceutically acceptable carrier.

18. A method of treating or prophylaxis of an AXL-, Mer- and/or c-Met-mediated disease in a subject, wherein the disease is selected from the group consisting of papillary thyroid carcinoma, pancreatic cancer, lung cancer, colon cancer, breast carcinoma, neuroblastoma, pain, cachexia, dermatitis, and asthma, the method comprising administering a pharmaceutically effective amount of the compound of any one of aspects 1-16 or a pharmaceutically acceptable salt thereof to a subject in need of such treatment.

19. The method of aspect 18, wherein the lung cancer is non-small cell lung cancer.

20. A method of inhibiting a AXL, Mer, and/or c-Met enzyme in a cell, the method comprising administering a pharmaceutically effective amount of the compound of any one of aspects 1-16 or a pharmaceutically acceptable salt thereof to a cell in need of such inhibition.

The following examples further illustrate the invention but, of course, should not be construed as in any way limiting its scope.

### EXAMPLES

NMR spectra were recorded in CDCl₃ and DMSO-d₆ solution in 5-mm o.d. tubes (Norell, Inc. 507-TIP) at 30° C. and were collected on Varian VNMRS-400 at 400 MHz for ¹H. The chemical shifts (6) are relative to tetramethylsilane (TMS=0.00 ppm) and expressed in ppm. LC/MS was taken on Ion-trap Mass Spectrometer on FINNIGAN Thermo LCQ Advantage MAX, Agilent LC 1200 series (Column: YMC Hydrosphere (C18, Ø 4.6×50 mm, 3 μm, 120 Å, 40° C.) operating in ESI (+) ionization mode; flow rate=1.0 mL/min., mobile phase=0.01% heptafluorobutyric acid (HFBA) and 1.0% isopropyl alcohol (IPA) in water or CH₃CN.

### Intermediate Example 1

This example describes the synthesis of 1-morpholinoprop-2-yn-1-one (Intermediate 1).

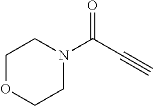

n-BuLi (2.5 M in hexane, 4.89 mL, 12.22 mmol) was slowly added to a solution of ethynyltrimethylsilane (1.45 mL, 10.18 mmol) in tetrahydrofuran (THF) (50 mL) at −78° C. The reaction mixture was stirred for 1 h at the same temperature and was added morpholine-4-carbonyl chloride (1.27 mL, 11.20 mmol). The reaction mixture was stirred additionally for 2 h at room temperature (rt). Water was added to the reaction mixture and stirred for 10 min. Ethyl acetate (EtOAc) was poured into the mixture and the separated organic layer was extracted with EtOAc. The organic layer was dried over Na₂SO₄, filtered, and concentrated in vacuo. The residue was purified by column chromatography on SiO₂ (Hexanes/EtOAc=1/1) to afford the 1-morpholinoprop-2-yn-1-one (1.03 g, 73%) as an off-white solid. ¹H-NMR (CDCl₃, Varian, 400 MHz): δ 3.14 (1H, s), 3.64-3.69 (4H, m), 3.70-3.73 (2H, m), 3.77-3.79 (2H, m).

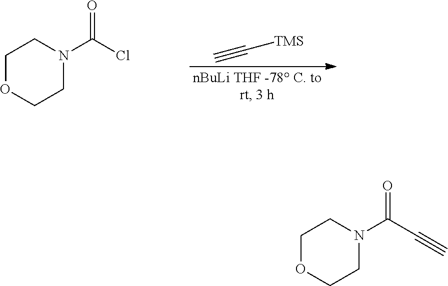

### Intermediate Example 2

This example describes the synthesis of 4-(prop-2-ynyl)morpholine (Intermediate 2).

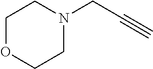

To a solution of morpholine (0.50 g, 5.74 mmol) in acetone (30.0 mL) were added 3-bromoprop-1-yne (0.82 g, 6.89 mmol) and potassium carbonate (1.03 g, 7.46 mmol). The reaction mixture was stirred for 8 h at room temperature. The mixture was filtered through a CELITE™ pad (Sigma-Aldrich, St. Louis, Mo.), and the filtrate was concentrated in vacuo. The residue was purified by column chromatography on SiO₂ (Hexanes/EtOAc=1/1) to afford the 4-(prop-2-ynyl)morpholine (370 mg, 52%) as a yellow oil. ¹H-NMR (CDCl₃, Varian, 400 MHz): δ 2.27 (1H, s), 2.57 (4H, t, J=4.8 Hz), 3.29 (2H, t, J=2.0 Hz), 3.74 (4H, t, J=4.4 Hz).

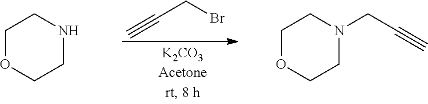

### Intermediate Example 3

This example describes the synthesis of 1-morpholinoprop-2-en-1-one (Intermediate 3).

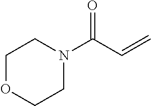

A mixture of acryloyl chloride (0.50 g, 5.52 mmol) and morpholine (0.96 g, 11.05 mmol) in dichloromethane (DCM) (10 mL) was stirred overnight at room temperature. The reaction mixture was diluted with DCM and water. The separated aqueous layer was extracted with DCM. The combined organic layer was dried over Na₂SO₄, filtered, and concentrated in vacuo to afford the 1-morpholinoprop-2-en-1-one (0.70 g, 90%) as an oil, which was used to next step without further purification. ¹H-NMR (CDCl₃, Varian, 400 MHz): δ 3.58-3.70 (8H, m), 5.71-5.74 (1H, m), 6.29-6.33 (1H, m), 6.53-6.60 (1H, m).

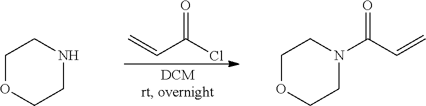

### Intermediate Example 4

This example describes the synthesis of tert-butyl 4-(prop-2-ynyl)piperazine-1-carboxylate (Intermediate 4).

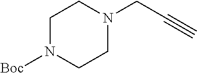 [SMILES: C#CCN1CCN(C(=O)OC(C)(C)C)CC1]

To a mixture of a tert-butyl piperazine-1-carboxylate (5.00 g, 26.8 mmol) and K₂CO₃ (7.42 g, 53.7 mmol) in CH₃CN (140 mL) was added dropwise 3-bromoprop-1-yne (2.63 mL, 34.9 mmol) at 0° C. The reaction mixture was stirred for 2 h at room temperature. The reaction mixture was filtered through a CELITE™ pad, and the filtrate was concentrated in vacuo. The residue was purified by column chromatography (Hexanes/EtOAc=1/1) to afford the tert-butyl 4-(prop-2-ynyl)piperazine-1-carboxylate (5.39 g, 90%) as a yellow oil. ¹H-NMR (CDCl₃, Varian, 400 MHz): δ 1.46 (9H, s), 2.26 (1H, brs), 2.51 (4H, brs), 3.32 (2H, s), 3.47 (4H, t, J=4.4 Hz).

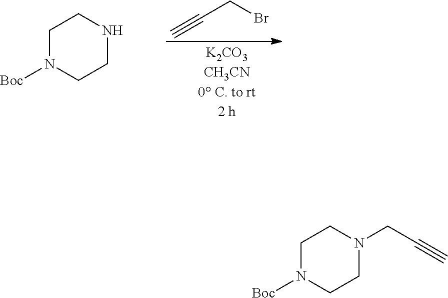

### Intermediate Example 5

This example describes the synthesis of tert-butyl 4-propioloylpiperazine-1-carboxylate (Intermediate 5).

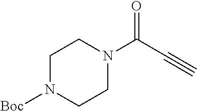 [SMILES: C#CC(=O)N1CCN(C(=O)OC(C)(C)C)CC1]

To a solution of propiolic acid (0.67 g, 9.66 mmol) in DCM (22 mL) was added N,N-dicyclohexylcarbodiimide (DCC) (1.76 mL, 9.66 mmol) at −5° C. and stirred for 1 h. To the reaction mixture were added tert-butyl piperazine-1-carboxylate (2.0 g, 10.74 mmol) and N,N-diisopropylethylamine (DIPEA) (5.75 mL, 32.2 mmol). The reaction mixture was stirred for 1 h at room temperature. The reaction mixture was filtered through a CELITE™ pad, and the filtrate was concentrated in vacuo. The residue was purified by column chromatography on SiO₂ (Hexanes/EtOAc=1/1) to afford the tert-butyl 4-propioloylpiperazine-1-carboxylate (1.09 g, 43%) as a white solid. ¹H-NMR (CDCl₃, Varian, 400 MHz): δ 1.47 (9H, s), 3.15 (1H, s), 3.43 (2H, t, J=5.6 Hz), 3.49 (2H, t, J=5.6 Hz), 3.61 (2H, t, J=5.6 Hz), 3.74 (2H, t, J=5.6 Hz).

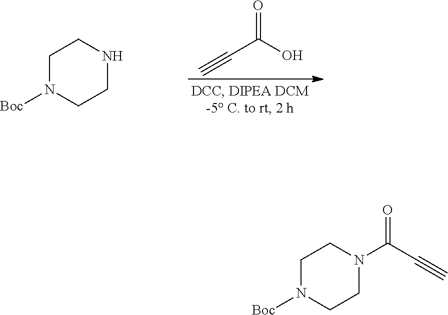

### Intermediate Example 6

This example describes the synthesis of tert-butyl 4-acryloylpiperazine-1-carboxylate (Intermediate 6).

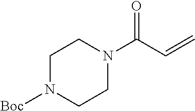 [SMILES: C=CC(=O)N1CCN(C(=O)OC(C)(C)C)CC1]

Triethylamine (TEA) (1.39 mL, 10.0 mmol) was added to a solution of acryloyl chloride (0.89 mL, 11.0 mmol) and tert-butyl piperazine-1-carboxylate in DCM (60 mL) at 0° C. The reaction mixture was stirred for 2 h at room temperature. The reaction mixture was washed with water and saturated NaHCO₃ (aq.). The organic layer was dried over Na₂SO₄, filtered, and concentrated in vacuo to afford the tert-butyl 4-acryloylpiperazine-1-carboxylate (2.33 g, 97%) as a pale yellow solid. ¹H-NMR (CDCl₃, Varian, 400 MHz): δ 1.37 (9H, s), 3.35 (8H, brs), 5.36 (1H, dd, J=10.4 Hz), 6.19 (1H, dd, J=16.8 Hz), 6.48 (1H, dd, J=16.8 Hz).

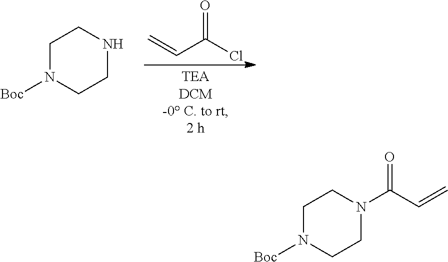

### Intermediate Example 7

This example describes the synthesis of 1-(4-hydroxypiperidin-1-yl)prop-2-yn-1-one (Intermediate 7).

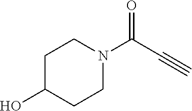

A mixture of piperidin-4-ol (1.00 g, 9.89 mmol), propiolic acid (1.04 g, 14.8 mmol), hexafluorophosphate (HATU) (5.64 g, 14.8 mmol), and TEA (5.51 mL, 39.5 mmol) in dimethylfuran (DMF) (15 mL) was stirred overnight at room temperature. The mixture was partitioned between EtOAc and water, the separated organic layer was dried over Na₂SO₄, filtered, and concentrated in vacuo. The residue was purified by column chromatography on SiO₂ (EtOAc) to afford the 1-(4-hydroxypiperidin-1-yl)prop-2-yn-1-one (663 mg, 44%) as a pale yellow oil. ¹H-NMR (CDCl₃, Varian, 400 MHz): δ 1.26-1.37 (2H, m), 1.68-1.76 (2H, m), 3.15 (1H, m), 3.43 (1H, m), 3.72 (1H, m), 3.81 (1H, m), 3.92 (1H, m), 4.51 (1H, s), 4.82 (1H, d, J=4.0 Hz).

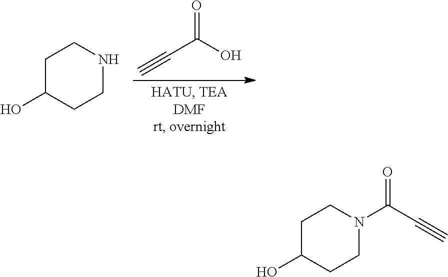

### Intermediate Example 8

This example describes the synthesis of 1-(4-methoxypiperidin-1-yl)prop-2-yn-1-one (Intermediate 8).

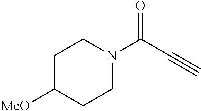

### Step A: tert-Butyl 4-hydroxypiperidine-1-carboxylate

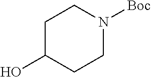 [SMILES: CC(C)(C)OC(=O)N1CCC(O)CC1]

To a solution of piperidin-4-ol (2.00 g, 19.8 mmol) in DCM (55 mL) were added Boc₂O (4.80 g, 21.8 mmol) and Na₂CO₃ (4.4 g, 41.5 mmol) in H₂O (70 mL). The reaction mixture was stirred for 3 days at room temperature. DCM and water were poured into the reaction mixture and the separated aqueous layer was extracted with DCM. The combined organic layers were dried over Na₂SO₄, filtered, and concentrated in vacuo, to afford the tert-butyl 4-hydroxypiperidine-1-carboxylate (3.98 g, 100%) as a colorless oil. ¹H-NMR (CDCl₃, Varian, 400 MHz): δ 1.46 (9H, s), 1.64 (1H, d, J=4.4 Hz), 1.84-1.86 (2H, m), 2.99-3.06 (2H, m), 3.81-3.86 (4H, m). *OH peak was not observed.

### Step B: tert-Butyl 4-methoxypiperidine-1-carboxylate

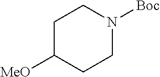 [SMILES: COC1CCN(C(=O)OC(C)(C)C)CC1]

A mixture of tert-butyl 4-hydroxypiperidine-1-carboxylate (3.98 g, 19.8 mmol) and KOH (2.22 g, 19.9 mmol) in DMSO (16 mL) was stirred for 1 h at room temperature, and then iodomethane (1.36 mL, 21.8 mmol) was added to the mixture. The reaction mixture was stirred for 4 h at room temperature. DCM and water were poured into the reaction mixture, and the separated aqueous layer was extracted with DCM. The combined organic layer was dried over Na₂SO₄, filtered, and concentrated in vacuo to afford the tert-butyl 4-methoxypiperidine-1-carboxylate (4.26 g, 100%) as a colorless liquid. ¹H-NMR (CDCl₃, Varian, 400 MHz): δ 1.46 (9H, s), 1.84 (2H, s), 2.62 (3H, s), 3.00-3.11 (2H, m), 3.35 (2H, s), 3.76-3.84 (3H, m).

### Step C: 4-Methoxypiperidine

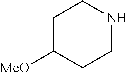

To a suspension of tert-butyl 4-methoxypiperidine-1-carboxylate (4.26 g, 19.3 mmol) in DCM (50 mL) was added trifluoroacetic acid (TFA) (6.10 mL, 79.0 mmol) at 0° C. The reaction mixture was stirred for 2 h at room temperature, and concentrated in vacuo to afford the 4-methoxypiperidine (2.27 g, 100%) as a yellow oil. ¹H-NMR (DMSO-d₆, Varian, 400 MHz): δ 1.92-2.12 (5H, m), 2.63 (3H, s), 3.07-3.10 (1H, m), 3.26 (1H, m), 3.34 (1H, s), 3.54 (1H, s), 9.31 (1H, s).

### Step D: 1-(4-Methoxypiperidin-1-yl)prop-2-yn-1-one

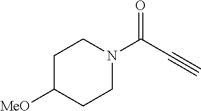

A mixture of 4-methoxypiperidine (300 mg, 2.60 mmol), propiolic acid (274 mg, 3.91 mmol), HATU (1.48 g, 3.91 mmol), and TEA (1.5 mL, 10.4 mmol) in DMF (5 mL) was stirred overnight at room temperature. The mixture was partitioned between EtOAc and water, the separated organic layer was dried over Na₂SO₄, filtered, and concentrated in vacuo. The residue was purified by column chromatography on SiO₂ (EtOAc) to afford the 1-(4-methoxypiperidin-1-yl)prop-2-yn-1-one (40.4 mg, 10%) as a pale yellow oil. ¹H-NMR (DMSO-d₆, Varian, 400 MHz): δ 1.61-1.71 (2H, m), 1.80-1.90 (2H, m), 3.13 (1H, s), 3.37 (3H, s), 3.49-3.54 (2H, m), 3.60-3.67 (1H, m), 3.76-3.83 (1H, m), 3.90-3.97 (1H, m).

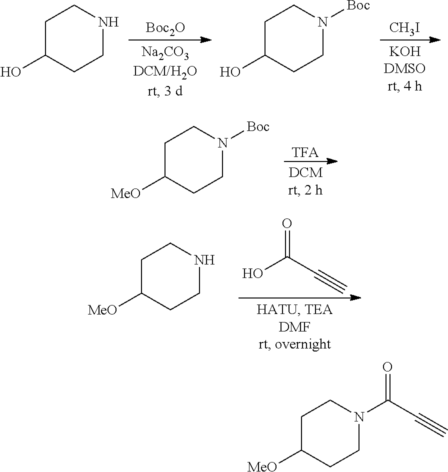

### Intermediate Example 9

This example describes the synthesis of 4-(but-3-ynyl)morpholine (Intermediate 9).

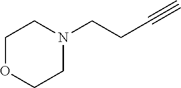

A mixture of 4-bromobut-1-yne (2.0 g, 15.04 mmol) and morpholine (2.62 g, 30.1 mmol) was heated for 1 h at 100° C. The mixture was diluted with Et₂O (15 mL) and filtered. The filtrate was extracted with 3N HCl. The aqueous layer was basified with saturated NaOH (aq.) and back-extracted into EtOAc. The organic layer was dried over Na₂SO₄, filtered, and concentrated in vacuo to afford the 4-(but-3-ynyl)morpholine (1.37 g, 65%) as a colorless oil. ¹H-NMR (CDCl₃, Varian, 400 MHz): δ 2.04 (1H, s), 2.37-2.39 (2H, m), 2.47 (4H, s), 2.59 (2H, d, J=7.6 Hz), 3.70 (4H, s).

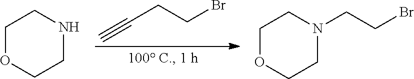

### Intermediate Example 10

This example describes the synthesis of N-(2-Methoxyethoxy)propiolamide (Intermediate 10).

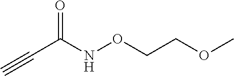

### Step A: 2-(2-Methoxyethoxy)isoindoline-1,3-dione

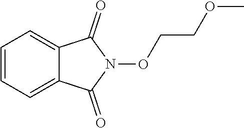 [SMILES: COCCON1C(=O)c2ccccc2C1=O]

To a mixture of 2-hydroxyisoindoline-1,3-dione (5.00 g, 65.7 mmol), 2-methoxyethanol (11.8 g, 72.3 mmol) and PPh₃ (19.0 g, 72.3 mmol) in THE (85 mL) was added dropwise diisopropyl azodicarboxylate (DIAD) (16.6 mL, 85.0 mmol) at 0° C. The reaction mixture was stirred overnight at room temperature and then concentrated in vacuo. The residue was purified by column chromatography on SiO₂ (Hexanes/EtOAc=7/3) to afford the 2-(2-methoxyethoxy)isoindoline-1,3-dione (16.4 g, quant.) as a white solid. ¹H-NMR (CDCl₃, Varian, 400 MHz): δ 3.39 (3H, s), 3.75-3.77 (2H, m), 4.36-4.38 (2H, m), 7.74-7.77 (2H, m), 7.84-7.86 (2H, m).

### Step B: O-(2-Methoxyethyl)hydroxylamine

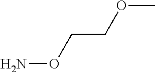

To a solution of 2-(2-methoxyethoxy)isoindoline-1,3-dione (14.5 g, 65.7 mmol) in EtOAc (131 mL) was added ethanolamine (4.37 mL, 72.3 mmol) at room temperature. The reaction mixture was stirred for 2 h at 80° C. and then concentrated in vacuo. The residue was triturated with Et₂O and isopropyl ether (IPE), and the solid was collected by filtration. The filtrate was concentrated in vacuo to afford the O-(2-methoxyethyl)hydroxylamine (1.06 g, 18%) as a yellow oil. ¹H-NMR (CDCl₃, Varian, 400 MHz): δ 3.39 (3H, s), 3.56-3.59 (2H, m), 3.82-3.85 (2H, m), 5.53 (2H, brs).

### Step C: N-(2-Methoxyethoxy)propiolamide

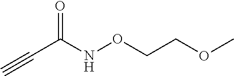

A mixture of O-(2-methoxyethyl)hydroxylamine (390 mg, 4.28 mmol) and propiolic acid (100 mg, 1.43 mmol) in THE (4 mL) was added dropwise to a solution of DCC (442 mg, 2.14 mmol) in THF (3 mL) at 0° C. The reaction mixture was stirred for 3 h at room temperature and filtered through a CELITE™ pad. The filtrate was concentrated in vacuo, and the residue was purified by column chromatography on SiO₂ (Hexanes/EtOAc=2/1) to afford the N-(2-methoxyethoxy)propiolamide (128 mg, 63%) as a yellow oil. ¹H-NMR (CDCl₃, Varian, 400 MHz): δ 2.88 (1H, brs), 3.42 (3H, s), 3.66-3.68 (2H, m), 4.11-4.13 (2H, m), 8.91 (1H, brs).

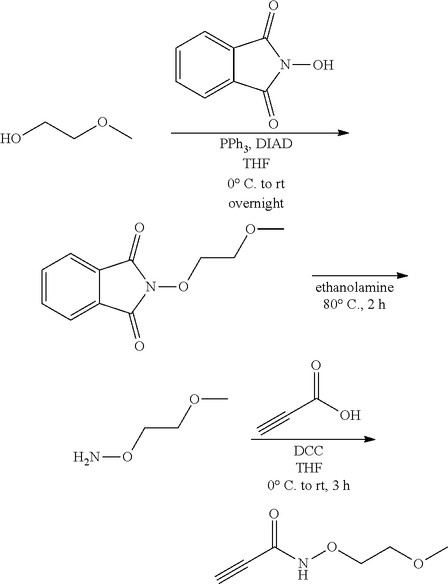

### Intermediate Example 11

This example describes the synthesis of N-(2-Methoxyethoxy)-N-methylpropiolamide (Intermediate 11).

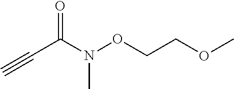

### Step A: Ethyl hydroxy(methyl)carbamate

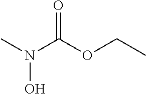

To a solution of N-methylhydroxylamine hydrochloride (1.00 g, 12.0 mmol) in THF/H₂O (v/v=10/1, 24.2 mL) were added NaHCO₃ (2.00 g, 24.0 mmol) and ethyl chloroformate (1.25 mL, 13.2 mmol) at room temperature. The reaction mixture was stirred overnight at room temperature. Water was poured into the reaction mixture and extracted with Et₂O. The combined organic layer was washed with brine, dried over Na₂SO₄, filtered, and concentrated in vacuo to afford the ethyl hydroxy(methyl)carbamate (1.39 g, 97%) as a colorless oil, which was used for the next step without further purification. ¹H-NMR (CDCl₃, Varian, 400 MHz): δ 1.29 (3H, t, J=8.0 Hz), 3.20 (3H, s), 4.19 (2H, q, J=8.0 Hz). * OH peak was not observed.

### Step B: Ethyl 2-methoxyethoxy(methyl)carbamate

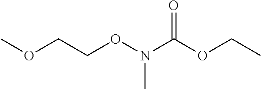

A mixture of ethyl hydroxy(methyl)carbamate (1.39 g, 11.7 mmol) and 1-bromo-2-methoxyethane (1.10 mL, 11.7 mmol) in EtOH (18 mL) was added dropwise a solution of KOH (687 mg, 12.2 mmol) in EtOH (7 mL) at room temperature. The reaction mixture was stirred overnight at 90° C., and filtered through a CELITE™ pad. The filtrate was concentrated in vacuo. The residue was partitioned between Et₂O and water and extracted with Et₂O. The combined organic layer was washed with saturated NH₄Cl (aq.), and dried over Na₂SO₄, filtered, and concentrated in vacuo. The residue was purified by column chromatography on SiO₂ (Hexanes/Et₂O=1/1 to 2/3) to afford the ethyl 2-methoxyethoxy(methyl)carbamate (765 mg, 37%) as a colorless oil. ¹H-NMR (CDCl₃, Varian, 400 MHz): δ 1.31 (3H, t, J=7.2 Hz), 3.17 (3H, s), 3.40 (3H, s), 3.59-3.61 (2H, m), 4.02-4.04 (2H, m), 4.17 (2H, q, J=7.2 Hz).

### Step C: O-(2-Methoxyethyl)-N-methylhydroxylamine

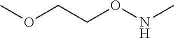

To a solution of ethyl 2-methoxyethoxy(methyl)carbamate (765 mg, 4.32 mmol) in EtOH/H₂O (v/v=1/1, 28.8 mL) was added KOH (969 mg, 17.3 mmol) at room temperature. The reaction mixture was stirred for 2 h at 40° C. The mixture was partitioned between Et₂O and water and extracted with Et₂O and DCM. The combined organic layer was dried over Na₂SO₄, filtered, and concentrated in vacuo to afford the O-(2-methoxyethyl)-N-methylhydroxylamine (368 mg, 81%) as a yellow oil, which was used for the next step without further purification. ¹H-NMR (CDCl₃, Varian, 400 MHz): δ 2.73 (3H, s), 3.39 (3H, s), 3.55-3.58 (2H, m), 3.84-3.86 (2H, m). * NH peak was not observed.

### Step D: N-(2-Methoxyethoxy)-N-methylpropiolamide

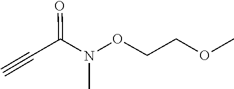

To a solution of O-(2-methoxyethyl)-N-methylhydroxylamine (300 mg, 2.86 mmol) and propiolic acid (100 mg, 1.43 mmol) in THE (4 mL) was added dropwise a solution of DCC (442 mg, 2.14 mmol) in THE (3 mL) at 0° C. The reaction mixture was stirred for 3 h at room temperature and filtered through a CELITE™ pad. The filtrate was concentrated in vacuo. The residue was purified by column chromatography on SiO₂ (Hexanes/Et₂O=1/4) to afford the N-(2-methoxyethoxy)-N-methylpropiolamide (153 mg, 68%) as a yellow oil. ¹H-NMR (CDCl₃, Varian, 400 MHz): δ 3.14 (1H, brs), 3.27 (3H, brs), 3.41 (3H, s), 3.63-3.67 (2H, m), 4.12-4.14 (2H, m).

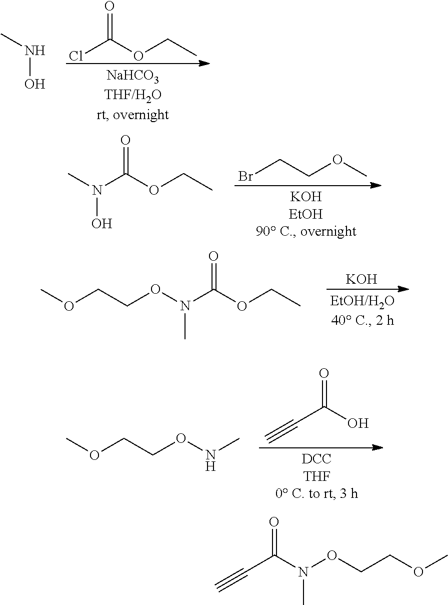

### Intermediate Example 12

This example describes the synthesis of tert-butyl 4-(2-methylbut-3-yn-2-yl)piperazine-1-carboxylate (Intermediate 12).

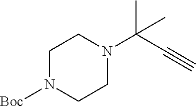 [SMILES: C#CC(C)(C)N1CCN(C(=O)OC(C)(C)C)CC1]

To a solution of tert-butyl piperazine-1-carboxylate (0.50 g, 2.68 mmol), 3-chloro-3-methylbut-1-yne (0.39 mL, 3.50 mmol) and TEA (0.48 mL, 3.50 mmol) in THE (10.0 mL) was added copper(I) chloride (0.02 g, 0.19 mmol) under N₂ atmosphere. The reaction mixture was stirred for 30 min at room temperature. Water-1N HCl (v/v=2/1, 3.0 mL) was poured into the mixture and extracted with EtOAc. The organic layer was dried over Na₂SO₄, filtered, and concentrated in vacuo to afford the tert-butyl 4-(2-methylbut-3-yn-2-yl)piperazine-1-carboxylate (0.66 g, 97%) as an ivory solid. ¹H-NMR (CDCl₃, Varian, 400 MHz): δ 1.39 (6H, s), 1.46 (9H, s), 2.29 (1H, s), 2.58 (4H, m), 3.44-3.46 (4H, m)

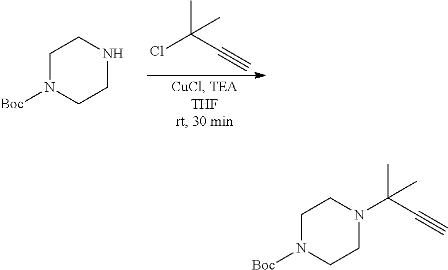

### Intermediate Example 13

This example describes the synthesis of 4-(2-methylbut-3-yn-2-yl)morpholine (Intermediate 13).

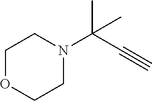

To a solution of morpholine (0.50 g, 5.74 mmol), 3-chloro-3-methylbut-1-yne (0.83 mL, 7.46 mmol), and TEA (1.0 mL, 7.46 mmol) in THE (10.0 mL) was added copper(I) chloride (0.04 g, 0.40 mmol) under N₂ atmosphere. The reaction mixture was stirred for 30 min at room temperature. Water-1N HCl (v/v=2/1, 3.0 mL) was poured into the mixture and extracted with EtOAc. The organic layer was dried over Na₂SO₄, filtered, and concentrated in vacuo to afford the 4-(2-methylbut-3-yn-2-yl)morpholine (0.66 g, 97%) as an ivory solid. ¹H-NMR (CDCl₃, Varian, 400 MHz): δ 1.38 (6H, s), 2.30 (1H, s), 2.62-2.64 (4H, m), 3.73-3.76 (4H, m).

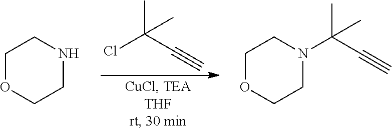

### Intermediate Example 14

This example describes the synthesis of 1-methyl-4-(prop-2-ynyl)piperazine (Intermediate 14).

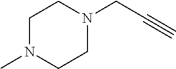

To a solution of 1-methylpiperazine (3.80 mL, 33.7 mmol) and K₂CO₃ (4.70 g, 33.7 mmol) in acetone (40 mL) was added a solution of 3-bromoprop-1-yne (1.70 mL, 22.5 mmol) in acetone (10 mL) at 0° C. The reaction mixture was stirred for 4 h at room temperature and concentrated in vacuo. Water was poured into the residue and extracted with DCM. The organic layer was dried over Na₂SO₄, filtered, and concentrated in vacuo to afford the 1-methyl-4-(prop-2-ynyl)piperazine (2.10 g, 45%) as a dark yellow oil. ¹H-NMR (CDCl₃, Varian, 400 MHz): δ 1.76 (1H, brs), 2.14-2.16 (1H, m), 2.19 (3H, s), 2.74-2.48 (8H, m), 3.17 (1H, d, J=2.4 Hz).

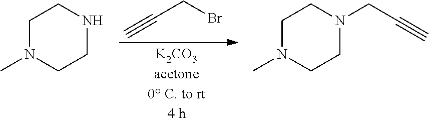

### Intermediate Example 15

This example describes the synthesis of 2-(4-fluorophenyl)-3-oxo-2,3-dihydropyridazine-4-carboxylic acid (Intermediate 15).

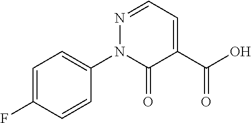 [SMILES: O=C(O)c1ccnn(-c2ccc(F)cc2)c1=O]

### Step A: (E)-2-(2-(4-Fluorophenyl)hydrazono)acetaldehyde

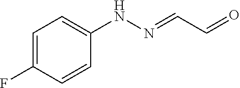 [SMILES: O=C/C=N/Nc1ccc(F)cc1]

To a solution of (4-fluorophenyl)hydrazine (10.0 g, 61.5 mmol) in H₂O/AcOH (v/v=1/1, 200 mL) was slowly added oxaldehyde (35.1 mL, 308 mmol) at room temperature over 30 min. After being stirred for 2 h, the reaction mixture was filtered and washed with water, and dried to afford the (E)-2-(2-(4-fluorophenyl)hydrazono)acetaldehyde (8.59 g, 84%) as a brown solid. ¹H-NMR (CDCl₃, Varian, 400 MHz): δ 7.06 (2H, t, J=8.6 Hz), 7.16-7.20 (2H, m), 7.24 (1H, d, J=8.0 Hz), 8.73 (1H, brs), 9.60 (1H, d, J=7.2 Hz).

### Step B: (E)-5-(2-(2-(4-Fluorophenyl)hydrazono)ethylidene)-2,2-dimethyl-1,3-dioxane-4,6-dione

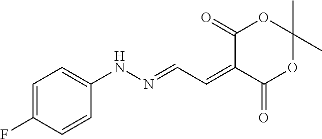 [SMILES: CC1(C)OC(=O)C(=C/C=N/Nc2ccc(F)cc2)C(=O)O1]

To a solution of (E)-2-(2-(4-fluorophenyl)hydrazono)acetaldehyde (8.59 g, 51.7 mmol), 2,2-dimethyl-1,3-dioxane-4,6-dione (7.45 g, 51.7 mmol) in toluene (172 mL) were added acetic acid (0.50 mL, 8.79 mmol) and piperidine (0.51 mL, 5.17 mmol) at room temperature. After being stirred for 3 days, the reaction mixture was filtered, washed with Et₂O, and dried to afford the (E)-5-(2-(2-(4-fluorophenyl)hydrazono)ethylidene)-2,2-dimethyl-1,3-dioxane-4,6-dione (13.1 g, 87%) as a red solid. ¹H-NMR (CDCl₃, Varian, 400 MHz): δ 1.77 (6H, s), 7.08 (2H, t, J=8.0 Hz), 7.34-7.38 (2H, m), 8.31 (1H, d, J=10.4 Hz), 8.90 (1H, d, J=10.4 Hz), 10.68 (1H, s).

### Step C: 2-(4-Fluorophenyl)-3-oxo-2,3-dihydropyridazine-4-carboxylic acid

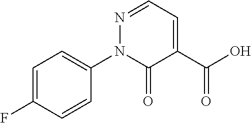 [SMILES: O=C(O)c1ccnn(-c2ccc(F)cc2)c1=O]

To a solution of (E)-5-(2-(2-(4-fluorophenyl)hydrazono)ethylidene)-2,2-dimethyl-1,3-dioxane-4,6-dione (1.00 g, 3.42 mmol) in MeOH (11.4 mL) was added sodium methoxide (0.20 g, 3.76 mmol) under N₂ atmosphere. After being stirred for 24 h at 75° C., the reaction mixture was cooled to room temperature. The reaction was quenched by 1N HCl at 0° C., extracted with DCM, dried over Na₂SO₄, filtered, and concentrated in vacuo. The residue was triturated with DCM and Et₂O to afford the 2-(4-fluorophenyl)-3-oxo-2,3-dihydropyridazine-4-carboxylic acid (668 mg, 83%) as a brown solid. ¹H-NMR (DMSO-d₆, Varian, 400 MHz): δ 7.37 (2H, t, J=8.4 Hz), 7.62 (2H, t, J=8.8 Hz), 7.97 (1H, d, J=4.0 Hz), 8.25 (1H, d, J=3.6 Hz), 13.7 (1H, s).

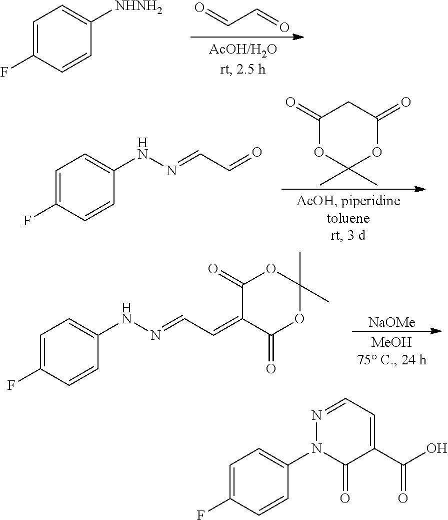

### Intermediate Example 16

This example describes the synthesis of 1-(4-fluorophenyl)-2-oxo-1,2-dihydropyridine-3-carboxylic acid (Intermediate 16).

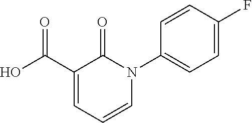 [SMILES: O=C(O)c1cccn(-c2ccc(F)cc2)c1=O]

### Step A: Methyl 1-(4-fluorophenyl)-2-oxo-1,2-dihydropyridine-3-carboxylate

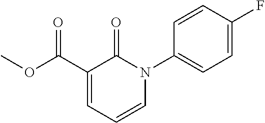 [SMILES: COC(=O)c1cccn(-c2ccc(F)cc2)c1=O]

To a solution of methyl 2-oxo-2H-pyran-3-carboxylate (300 mg, 1.95 mmol) in THF/DMF (v/v=4/1, 6.5 mL) was added 4-fluoroaniline (216 mg, 1.95 mmol) at room temperature. After being stirred for 3 h at room temperature, 1-ethyl-3-(3-dimethylaminopropyl)carbodiimide (EDC) (485 mg, 2.53 mmol) and 4-dimethylaminopyridine (DMAP) (24.0 mg, 0.195 mmol) were added to the mixture at room temperature. The reaction mixture was stirred overnight at room temperature, and quenched with 1N HCl. The aqueous layer was extracted with EtOAc, washed with water and brine, and dried over Na₂SO₄, filtered, and concentrated in vacuo. The residue was purified by column chromatography on SiO₂ (Hexanes/EtOAc=1/4) to afford the methyl 1-(4-fluorophenyl)-2-oxo-1,2-dihydropyridine-3-carboxylate (192 mg, 40%) as a yellow solid. ¹H-NMR (CDCl₃, Varian, 400 MHz): δ 3.91 (3H, s), 6.34 (1H, t, J=7.2 Hz), 7.18 (2H, t, J=8.4 Hz), 7.34-7.37 (2H, m), 7.56 (1H, dd, J=6.4, 2.0 Hz), 8.24 (1H, dd, J=7.2, 2.0 Hz).

### Step B: 1-(4-Fluorophenyl)-2-oxo-1,2-dihydropyridine-3-carboxylic acid

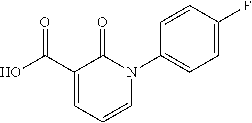 [SMILES: O=C(O)c1cccn(-c2ccc(F)cc2)c1=O]

To a solution of methyl 1-(4-fluorophenyl)-2-oxo-1,2-dihydropyridine-3-carboxylate (192 mg, 0.78 mmol) in MeOH (3.9 mL) was added 6 N NaOH (194 μL, 1.17 mmol) at room temperature. After being stirred for 5 h at room temperature, the reaction mixture was concentrated in vacuo. The residue was partitioned between water and EtOAc. The aqueous layer was acidified with 6N HCl until pH 3. The precipitated solid was collected by filtration, washed with water, and dried under vacuum to afford the 1-(4-fluorophenyl)-2-oxo-1,2-dihydropyridine-3-carboxylic acid (142 mg, 79%) as a yellow solid. ¹H-NMR (CDCl₃, Varian, 400 MHz): δ 6.65 (1H, t, J=7.2 Hz), 7.24 (2H, d, J=8.2 Hz), 7.37-7.40 (2H, m), 7.66 (1H, dd, J=6.8, 1.6 Hz), 8.61 (1H, dd, J=7.6, 1.6 Hz), 13.89 (1H, brs).

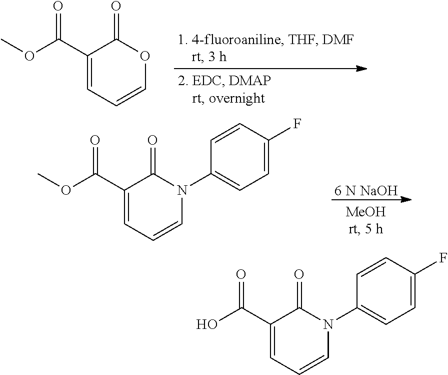

### Intermediate Example 17

This example describes the synthesis of 1-(4-fluorophenyl)-4-iodo-2-oxo-1,2-dihydropyridine-3-carboxylic acid (Intermediate 17).

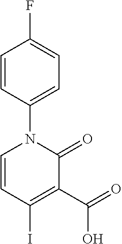 [SMILES: O=C(O)c1c(I)ccn(-c2ccc(F)cc2)c1=O]

### Step A: 2-Fluoro-3-iodopyridine

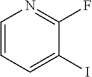

To a solution of 2-fluoropyridine (0.88 mL, 10.3 mmol) in THE (52 mL) was added slowly lithium diisopropylamide (LDA) (2 M in THF, 7.72 mL, 15.4 mmol) at −70° C. The mixture was stirred for 2 h at −70° C., and then iodine (3.92 g, 15.4 mmol) in THE (10 mL) was added. After the addition was completed, the reaction mixture was stirred for 1 h at −70° C., and then allowed to room temperature. The mixture was treated with a solution of sodium hydrogensulfite (10 g) in H₂O (60 mL) and stirred for 30 min and then extracted with EtOAc. The combined organic layer was washed with water and brine, dried over Na₂SO₄, and concentrated in vacuo. The residue was purified by column chromatography on SiO₂ (Hexanes/EtOAc=9/1) to afford the 2-fluoro-3-iodopyridine (1.14 g, 50%) as a yellow solid. ¹H-NMR (CDCl₃, Varian, 400 MHz): δ 6.98 (1H, t, J=5.6 Hz), 8.15-7.19 (2H, m).

### Step B: 4-Iodo-2-methyoxynicothaldehyde

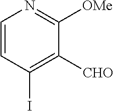 [SMILES: COc1nccc(I)c1C=O]

To a solution of 2-fluoro-3-iodopyridine (1.14 g, 5.10 mmol) in THE (15 mL) was added slowly LDA (2 M in THF, 3.31 mL, 6.63 mmol) at −70° C. The mixture was stirred for 2 h at −60° C. Ethyl formate (0.46 mL, 5.61 mmol) was added in dropwise manner at −70° C. After the addition was completed, the reaction mixture was added sodium methoxide (0.33 g, 6.12 mmol) in MeOH (11 mL), and then allowed to warm up to room temperature. The mixture was quenched by water and extracted with EtOAc. The combined organic layer was washed with brine, dried over Na₂SO₄, filtered, and concentrated in vacuo. The residue was purified by column chromatography on SiO₂ (Hexanes/EtOAc=9/1) to afford the 4-iodo-2-methyoxynicothaldehyde (0.57 g, 43%) as a yellow solid. ¹H-NMR (CDCl₃, Varian, 400 MHz): δ 4.05 (3H, s), 7.54 (1H, d, J=5.6 Hz), 7.85 (1H, d, J=5.6 Hz), 10.21 (1H, s).

### Step C: 4-Iodo-2-oxo-1,2-dihydropyridine-3-carbaldehyde

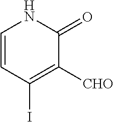

Chlorotrimethylsilane (0.42 mL, 3.33 mmol) was slowly added to a mixture of 4-iodo-2-methoxynicotinaldehyde (0.30 g, 1.11 mmol) and sodium iodide (0.50 g, 3.33 mmol) in CH₃CN (6.0 mL). The reaction mixture was stirred for 1 h at 30° C. and then concentrated in vacuo. EtOAc, water, and saturated NaHCO₃ were poured into the residue and the resulting suspension was filtered to give a dark brown solid. The solid was triturated with CH₃CN to afford the 4-iodo-2-oxo-1,2-dihydropyridine-3-carbaldehyde (0.25 g, 91%) as a dark brown solid. ¹H-NMR (DMSO-d₆, Varian, 400 MHz): δ 7.48 (1H, brs), 7.76 (1H, d, J=6.0 Hz), 9.84 (1H, s), 9.88 (1H, s).

### Step D: 1-(4-Fluorophenyl)-4-iodo-2-oxo-1,2-dihydropyridine-3-carbaldehyde

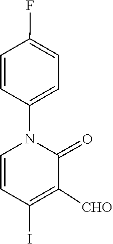 [SMILES: O=Cc1c(I)ccn(-c2ccc(F)cc2)c1=O]

To a mixture of 4-iodo-2-oxo-1,2-dihydropyridine-3-carbaldehyde (0.25 g, 1.01 mmol), 4-fluorophenylboronic acid (0.42 g, 3.04 mmol), copper(II) acetate (0.36 g, 2.03 mmol), and tetradecanoic acid (0.93 g, 4.05 mmol) in toluene (10 mL) was added lutidine (0.93 mL, 8.11 mmol) at room temperature. The reaction mixture was stirred for 40 h at room temperature and then quenched with 1N HCl. The aqueous layer was extracted with EtOAc, the combined organic layer was washed with brine, and dried over Na₂SO₄, filtered, and concentrated in vacuo. The residue was purified by column chromatography on SiO₂ (Hexanes/EtOAc=7/3) to afford the 1-(4-fluorophenyl)-4-iodo-2-oxo-1,2-dihydropyridine-3-carbaldehyde (58.0 mg, 17%) as a yellow solid. ¹H-NMR (CDCl₃, Varian, 400 MHz): δ 6.96 (1H, d, J=7.2 Hz), 7.14 (1H, d, J=7.2 Hz), 7.21 (2H, d, J=8.4 Hz), 7.35-7.38 (2H, m), 10.15 (1H, s).

### Step E: 1-(4-Fluorophenyl)-4-iodo-2-oxo-1,2-dihydropyridine-3-carboxylic acid

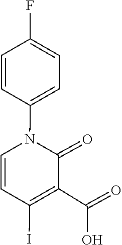 [SMILES: O=C(O)c1c(I)ccn(-c2ccc(F)cc2)c1=O]

To a mixture of 1-(4-fluorophenyl)-4-iodo-2-oxo-1,2-dihydropyridine-3-carbaldehyde (59 mg, 0.17 mmol) and sodium dihydrogen phosphate (51 mg, 0.42 mmol) in THF/t-BuOH/H₂O (v/v/v=1/1/1, 1.5 mL) were added 2-methyl-2-butene (0.13 mL, 0.25 mmol) and sodium chlorite (36 mg, 0.39 mmol) at 0° C. The reaction mixture was stirred for 1 h at room temperature, and then quenched with 1N HCl (2 mL). The solid was collected by filtration, washed with water and Et₂O, dried under vacuum to afford the 1-(4-fluorophenyl)-4-iodo-2-oxo-1,2-dihydropyridine-3-carboxylic acid (48 mg, 78%) as a yellow solid. ¹H-NMR (DMSO-d₆, Varian, 400 MHz): δ 6.80 (1H, d, J=7.2 Hz), 7.36 (2H, t, J=8.8 Hz), 7.47-7.51 (3H, m), 13.50 (1H, s).

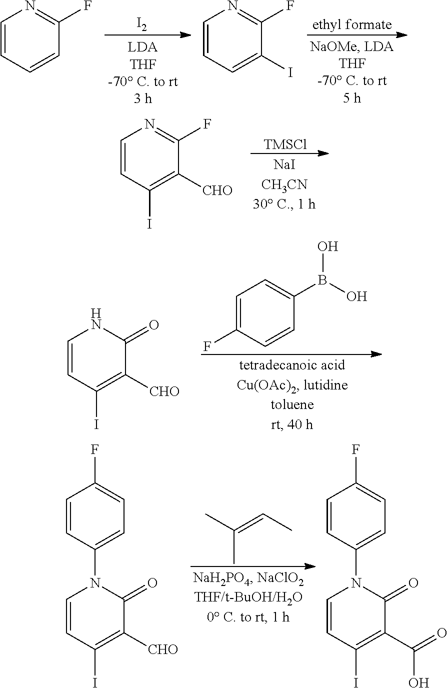

### Intermediate Example 18

This example describes the synthesis of 4-(4-fluorophenyl)-3-oxo-3,4-dihydropyrazine-2-carboxylic acid (Intermediate 18).

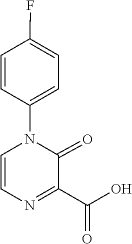 [SMILES: O=C(O)c1nccn(-c2ccc(F)cc2)c1=O]

### Step A: 2-(4-Fluorophenylamino)acetonitrile hydrochloride

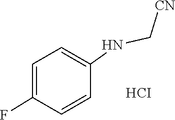

To a solution of 2-(4-fluorophenylamino)acetonitrile (2.23 g, 20.1 mmol) in glacial acetic acid (25 mL) were added portionwise paraformaldehyde (1.63 g, 54.2 mmol) and potassium cyanide (1.57 g, 24.1 mmol) at 0° C. The mixture was allowed to stir overnight at room temperature. The mixture was neutralized with saturated NaHCO₃(aq.) and extracted with EtOAc. The organic layer was dried and concentrated in vacuo. The resulting residue was purified by chromatography on SiO₂ (Hexanes/EtOAc=1/4) to afford the 2-(4-fluorophenylamino)acetonitrile (3.05 g, 100%) as a yellow oil. HCl (4 M in 1,4-dioxane, 25.4 mL, 102 mmol) was added to a solution of above obtained the 2-(4-fluorophenylamino)acetonitrile (3.05 g, 20.3 mmol) in 1,4-dioxane (50 mL), and the mixture was stirred overnight at room temperature. The solvent was concentrated in vacuo, and acetone was poured into the residue and the generated solid was collected by filtration to afford the 2-(4-fluorophenylamino)acetonitrile hydrochloride (2.44 g, 64%) as an off-white solid. ¹H-NMR (DMSO-d₆, Varian, 400 MHz): 4.40 (2H, s), 6.68 (2H, q, J=4.4 Hz), 6.98 (2H, t, J=9.8 Hz), 8.08 (1H, brs). * NH peak was not observed.

### Step B: 3,5-Dichloro-1-(4-fluorophenyl)pyrazin-2(1H)-one

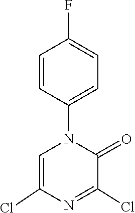 [SMILES: O=c1c(Cl)nc(Cl)cn1-c1ccc(F)cc1]

Oxalyl chloride was dropwise added to a solution of 2-(4-fluorophenylamino)acetonitrile hydrochloride (100 mg, 15.6 mmol) in dry toluene (50 mL) at 0° C. under N₂ atmosphere. After stirring at the same temperature for 45 min, triethylamine hydrochloride (3.22 g, 23.4 mmol) was added in small portions, and followed by addition of DMF (0.1 mL, 1.56 mmol). The reaction mixture was kept stirring for 2 days at room temperature. The reaction mixture was concentrated in vacuo and the residue was purified by column chromatography on SiO₂ (Hexanes/EtOAc=4/1) to afford the 3,5-dichloro-1-(4-fluorophenyl)pyrazin-2(1H)-one (1.99 g, 50%) as a pale yellow solid. ¹H-NMR (CDCl₃, Varian, 400 MHz): δ 7.23 (2H, t, J=8.8 Hz), 7.30 (1H, s), 7.39-7.42 (2H, m).

### Step C: 5-Chloro-1-(4-fluorophenyl)-3-methoxypyrazin-2(1H)-one

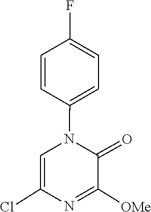 [SMILES: COc1nc(Cl)cn(-c2ccc(F)cc2)c1=O]

NaOMe (3.66 g, 19.2 mmol) was added to a solution of 3,5-dichloro-1-(4-fluorophenyl)pyrazin-2(1H)-one (1.99 g, 7.68 mmol) in MeOH (20 mL) at 0° C. The reaction mixture was stirred for 1 h at room temperature, neutralized with 2N HCl, and concentrated in vacuo. EtOAc and water were poured into the residue, and the separated aqueous layer was extracted with EtOAc. The combined organic layer was dried over Na₂SO₄, filtered, and concentrated in vacuo to afford the 5-chloro-1-(4-fluorophenyl)-3-methoxypyrazin-2(1H)-one (1.92 g, 98%) as a pale yellow solid. ¹H-NMR (CDCl₃, Varian, 400 MHz): δ 4.05 (3H, s), 6.95 (1H, s) 7.21 (2H, t, J=12.0 Hz), 7.37-7.40 (2H, m).

### Step D: 1-(4-Fluorophenyl)-3-methoxypyrazin-2(1H)-one

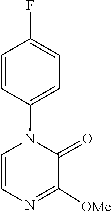 [SMILES: COc1nccn(-c2ccc(F)cc2)c1=O]

K₂CO₃ (1.04 g, 7.54 mmol) and 5% Pd/C (802 mg, 0.38 mmol) were added to a solution of 5-chloro-1-(4-fluorophenyl)-3-methoxypyrazin-2(1H)-one (1.92 g, 7.54 mmol) in MeOH (30 mL) at room temperature. The reaction was stirred for 6 h under H₂ atmosphere, and filtered through a CELITE™ pad, and concentrated in vacuo. The residue was treated with DCM, washed with water, dried over Na₂SO₄, filtered, and concentrated in vacuo to afford the 1-(4-fluorophenyl)-3-methoxypyrazin-2(1H)-one (1.11 g, 67%) as a colorless oil. ¹H-NMR (CDCl₃, Varian, 400 MHz): δ 4.01 (3H, s), 6.84 (1H, d, J=4.8 Hz), 6.90 (1H, d, J=4.8 Hz), 7.19 (2H, t, J=8.8 Hz), 7.39-7.42 (2H, m).

### Step E: 3-Chloro-1-(4-fluorophenyl)pyrazin-2(1H)-one

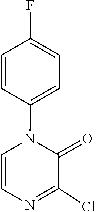 [SMILES: O=c1c(Cl)nccn1-c1ccc(F)cc1]

POCl₃ (1.18 mL, 12.6 mmol) was added dropwise to a solution of 1-(4-fluorophenyl)-3-methoxypyrazin-2(1H)-one (1.11 g, 5.04 mmol) in DMF (15 mL) at 0° C., and followed by heated for 1.5 h at 90° C. The reaction was quenched by addition of saturated NaOAc (aq.) at 0° C., and extracted with DCM. The combined organic layer was washed with water, dried over Na₂SO₄, filtered, and concentrated in vacuo. The residue was purified by column chromatography on NH—SiO₂ (DCM/MeOH=20/1) to afford the 3-chloro-1-(4-fluorophenyl)pyrazin-2(1H)-one (870 mg, 77%) as a white solid. ¹H-NMR (CDCl₃, Varian, 400 MHz): δ 7.17-7.26 (4H, m), 7.39-7.42 (2H, m).

### Step F: 4-(4-Fluorophenyl)-3-oxo-3,4-dihydropyrazine-2-carbonitrile

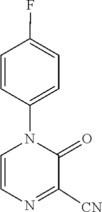 [SMILES: N#Cc1nccn(-c2ccc(F)cc2)c1=O]

A mixture of 3-chloro-1-(4-fluorophenyl)pyrazin-2(1H)-one (870 mg, 3.87 mmol), 1,1′-bis(diphenylphosphino)ferrocene (dppf) (215 mg, 0.39 mmol), Pd2(dba)₃ (177 mg, 0.19 mmol) and Zn(CN)₂ (273 mg, 2.32 mmol) in N-methylpyrrolidone (NMP) (10 mL) was heated for 15 h at 120° C. in a sealed vial. After cooling at room temperature, EtOAc and water were poured into the reaction mixture and the separated aqueous layer was extracted with EtOAc, dried over Na₂SO₄, filtered, and concentrated in vacuo. The residue was purified by column chromatography on NH—SiO₂ (DCM) to afford the 4-(4-fluorophenyl)-3-oxo-3,4-dihydropyrazine-2-carbonitrile (301 mg, 36%) as a yellow solid. ¹H-NMR (CDCl₃, Varian, 400 MHz): δ 7.18-7.28 (2H, m), 7.39-7.44 (2H, m), 7.46 (1H, d, J=4.0 Hz), 7.59 (1H, d, J=4.0 Hz).

### Step G: 4-(4-Fluorophenyl)-3-oxo-3,4-dihydropyrazine-2-carboxylic acid

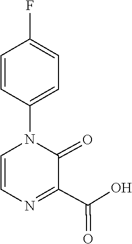 [SMILES: O=C(O)c1nccn(-c2ccc(F)cc2)c1=O]

A mixture of 4-(4-fluorophenyl)-3-oxo-3,4-dihydropyrazine-2-carbonitrile (301 mg, 1.40 mmol) and H₂SO₄ (3.00 mL, 56.0 mmol) was stirred for 17 h at room temperature. Then the mixture was added into MeOH (20 mL), and the reaction mixture was heated for 2.5 h at 70° C. The reaction was quenched with water and treated with 2N NaOH at 0° C. EtOAc and 2N HCl were poured into the reaction mixture and the separated aqueous layer was extracted with EtOAc. The combined organic layer was dried over Na₂SO₄, filtered, and concentrated in vacuo to afford the 4-(4-fluorophenyl)-3-oxo-3,4-dihydropyrazine-2-carboxylic acid (280 mg, 86%) as a yellow solid. ¹H-NMR (DMSO-d₆, Varian, 400 MHz): δ 7.42 (2H, d, J=8.4 Hz), 7.53 (1H, d, J=4.4 Hz), 7.59-7.62 (2H, m), 7.91 (1H, d, J=4.0 Hz). * OH peak was not observed.

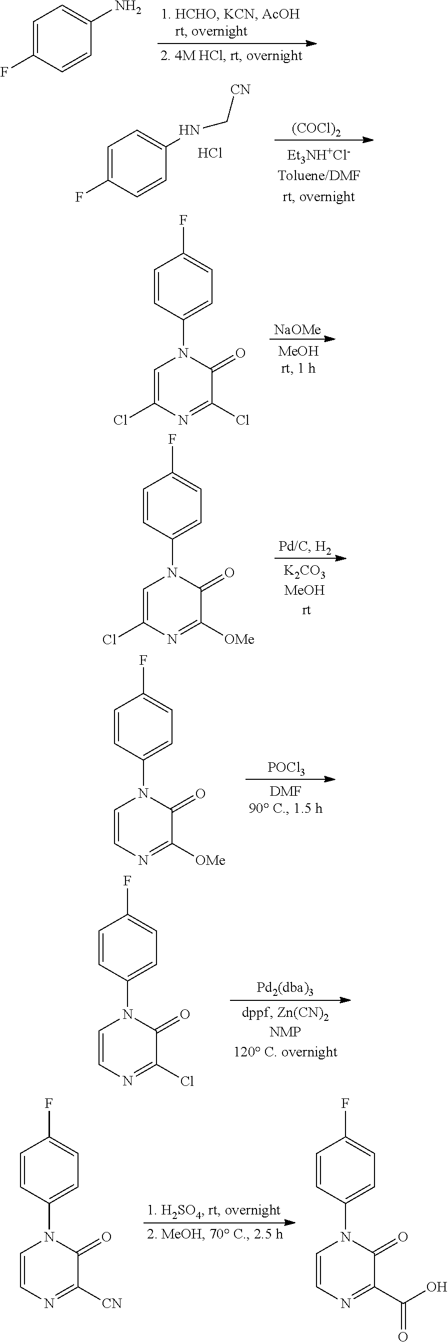

### Intermediate Example 19

This example describes the synthesis of N-(4-(2-amino-3-iodopyridin-4-yloxy)-3-fluorophenyl)-2-(4-fluorophenyl)-3-oxo-2,3-dihydropyridazine-4-carboxamide (Intermediate 19).

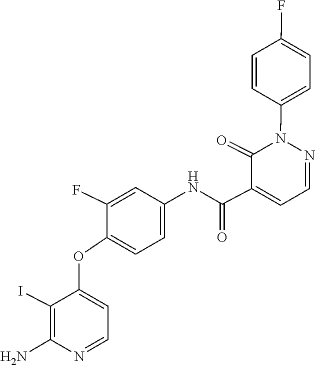 [SMILES: Nc1nccc(Oc2ccc(NC(=O)c3ccnn(-c4ccc(F)cc4)c3=O)cc2F)c1I]

### Step A: tert-Butyl 4-chloropyridin-2-ylcarbamate

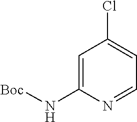 [SMILES: CC(C)(C)OC(=O)Nc1cc(Cl)ccn1]

To a solution of 4-chloropyridin-2-amine (3.00 g, 23.3 mmol) in THE (200 mL) was added sodium bis(trimethylsilyl)amide (NaHMDS) (1 M in THF, 46.7 mL, 46.7 mmol) at −10° C. A solution of di-tert-butyl dicarbonate (5.09 g, 23.34 mmol) in THE (10 mL) was then added at the same temperature. The reaction mixture was stirred for 16 h at room temperature. Saturated NH₄C₁ was added to the reaction mixture and the layers were separated. The aqueous phase was extracted with EtOAc. The combined organic layers were dried over Na₂SO₄, filtered, and concentrated in vacuo to afford the tert-butyl 4-chloropyridin-2-ylcarbamate (5.00 g, 94%) as a brown solid which was used for the next step without further purification. ¹H-NMR (CDCl₃, Varian, 400 MHz): δ 1.53 (9H, s), 6.95-6.97 (1H, m), 7.59 (1H, brs), 8.04 (1H, s), 8.13 (1H, d, J=5.6 Hz).

### Step B: tert-Butyl 4-chloro-3-iodopyridin-2-ylcarbamate

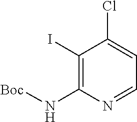 [SMILES: CC(C)(C)OC(=O)Nc1nccc(Cl)c1I]

n-BuLi (2 M in hexane, 8.75 mL, 21.9 mmol) was dropwise added to a solution of tert-butyl 4-chloropyridin-2-ylcarbamate (2.00 g, 8.75 mmol) and tetramethylethylenediamine (TMEDA) (3.27 mL, 21.87 mmol) in THE (292 mL) at −78° C. for 30 min. The mixture was stirred for 1 h at the same temperature, and then I₂ (11.1 g, 43.7 mmol) in THE (100 mL) was added. After the addition was completed, the reaction mixture was stirred for 30 min −78° C., and then allowed to warm up to room temperature. The mixture was treated with a solution of sodium hydrogensulfite (16.0 g) in H₂O (100 mL) and stirred for 30 min, and then extracted with EtOAc. The extracts were washed with brine, dried over Na₂SO₄, and concentrated in vacuo. The residue was purified by column chromatography on SiO₂ (Hexanes/EtOAc=1/1) to afford the tert-butyl 4-chloro-3-iodopyridin-2-ylcarbamate (2.10 g, 68%) as a white solid. ¹H-NMR (DMSO-d₆, Varian, 400 MHz): δ 1.45 (9H, s), 7.48 (1H, d, J=4.8 Hz), 8.30 (1H, d, J=4.8 Hz), 9.48 (1H, s).

### Step C: 4-Chloro-3-iodopyridin-2-amine

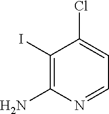

A suspension of tert-butyl 4-chloro-3-iodopyridin-2-ylcarbamate (2.10 g, 5.92 mmol) in HBr (10 mL, 5.92 mmol) was heated for 10 min at 0° C. to give a clear solution. After cooling at 0° C., the reaction mixture was treated with crushed ice and basified with 6 M NaOH (aq.). The precipitated product was collected by vacuum filtration, washed with water, and sucked partially on the funnel to give a white solid. The product was dissolved in THE and the solution dried over Na₂SO₄ and concentrated in vacuo to afford the 4-chloro-3-iodopyridin-2-amine (1.50 g, quant.) as a white solid. ¹H-NMR (DMSO-d₆, Varian, 400 MHz): δ 6.43 (2H, s), 6.72 (1H, d, J=5.2 Hz), 7.84 (1H, d, J=5.2 Hz).

### Step D: 4-(2-Fluoro-4-nitrophenoxy)-3-iodopyridin-2-amine

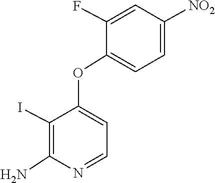 [SMILES: Nc1nccc(Oc2ccc([N+](=O)[O-])cc2F)c1I]

A mixture of 4-chloro-3-iodopyridin-2-amine (1.50 g, 5.89 mmol), 2-fluoro-4-nitrophenol (1.85 g, 11.8 mmol), DIPEA (1.54 mL, 8.84 mmol), and NMP (8 mL) was placed in a glass pressure vessel and heated rapidly to 170° C. The heating was continued for 18 h. After cooling at room temperature, the reaction mixture was dissolved with EtOAc and washed with saturated NaHCO₃ solution (aq.). The organic layer was dried over Na₂SO₄, filtered, and concentrated in vacuo. The residue was purified by column chromatography on SiO₂ (Hexanes/EtOAc=3/1) to afford the 4-(2-fluoro-4-nitrophenoxy)-3-iodopyridin-2-amine (1.48 g, 67%) as a pale yellow solid. 1H-NMR (DMSO-d₆, Varian, 400 MHz): δ 6.19 (1H, d, J=5.6 Hz), 6.41 (2H, s), 7.33 (1H, t, J=8.6 Hz), 7.87 (1H, d, J=5.6 Hz), 8.11-8.14 (1H, m), 8.40 (1H, dd, J=2.4, 10.4 Hz).

### Step E: 4-(4-Amino-2-fluorophenoxy)-3-iodopyridin-2-amine

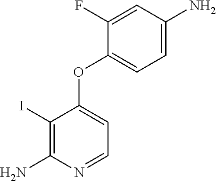 [SMILES: Nc1ccc(Oc2ccnc(N)c2I)c(F)c1]

A mixture of 4-(2-fluoro-4-nitrophenoxy)-3-iodopyridin-2-amine (150 mg, 0.40 mmol) and SnCl₂ (361 mg, 1.60 mmol) in EtOH (10 mL) was stirred vigorously for 2 h at 90° C. After cooling at room temperature, the solvent was removed under reduced pressure, EtOAc was poured into the residue. The mixture was neutralized with saturated NaHCO₃ (aq.) and 2 N NaOH until pH 9 and then filtered through a CELITE™ pad. The filtrate was extracted with EtOAc, dried over Na₂SO₄, and concentrated in vacuo to afford the 4-(4-amino-2-fluorophenoxy)-3-iodopyridin-2-amine (130 mg, 94%) as a yellow solid which was used to next step without further purification. 1H-NMR (CDCl₃, Varian, 400 MHz): δ 3.78 (2H, brs), 5.08 (2H, brs), 5.87 (1H, d, J=5.6 Hz), 6.44-6.53 (2H, m), 6.97 (1H, t, J=8.8 Hz), 7.76 (1H, d, J=5.6 Hz)

### Step F: N-(4-(2-Amino-3-iodopyridin-4-yloxy)-3-fluorophenyl)-2-(4-fluorophenyl)-3-oxo-2,3-dihydropyridazine-4-carboxamide

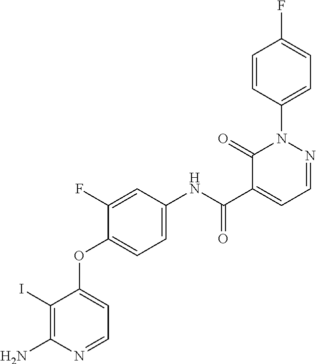 [SMILES: Nc1nccc(Oc2ccc(NC(=O)c3ccnn(-c4ccc(F)cc4)c3=O)cc2F)c1I]

A mixture of 4-(4-amino-2-fluorophenoxy)-3-iodopyridin-2-amine (200 mg, 0.58 mmol), 2-(4-fluorophenyl)-3-oxo-2,3-dihydropyridazine-4-carboxylic acid (intermediate 15, 204 mg, 0.87 mmol), HATU (242 mg, 0.64 mmol), and TEA (0.20 mL, 1.45 mmol) in DMF (5 mL) was stirred overnight at room temperature. Water was poured into the mixture, the precipitated product was collected by vacuum filtration, washed with water to afford the N-(4-(2-amino-3-iodopyridin-4-yloxy)-3-fluorophenyl)-2-(4-fluorophenyl)-3-oxo-2,3-dihydropyridazine-4-carboxamide (300 mg, 92%) as a brown solid. ¹H-NMR (CDCl₃, Varian, 400 MHz): δ 5.08 (2H, brs), 5.89 (1H, d, J=6.0 Hz), 7.16 (1H, t, J=8.6 Hz), 7.16-7.27 (2H, m), 7.58-7.61 (2H, m), 7.80 (1H, d, J=5.6 Hz), 7.90-7.93 (1H, m), 8.23 (1H, d, J=4.4 Hz), 8.41 (1H, d, J=4.4 Hz), 11.80 (1H, s). * NH peak was not observed.

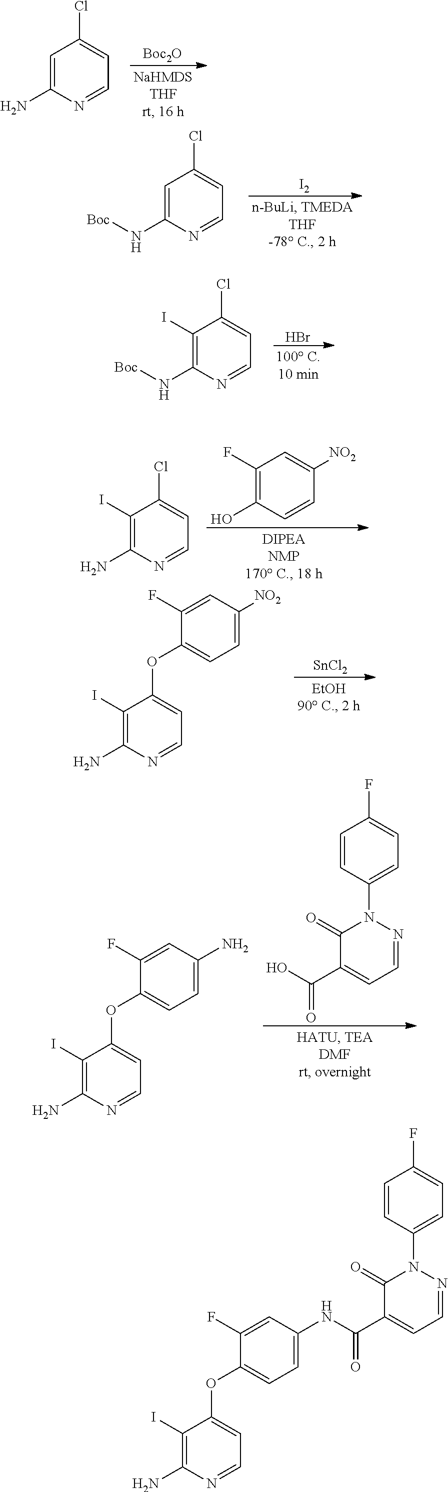

### Intermediate Example 20

This example describes the synthesis of tert-butyl 4-(3-(2-amino-4-(4-amino-2-fluorophenoxy)pyridin-3-yl)propioloyl)piperazine-1-carboxylate (Intermediate 20).

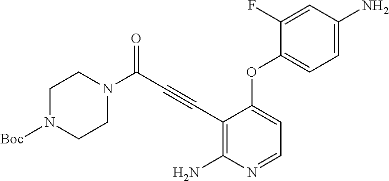 [SMILES: CC(C)(C)OC(=O)N1CCN(C(=O)C#Cc2c(Oc3ccc(N)cc3F)ccnc2N)CC1]

### Step A: tert-Butyl 4-(3-(2-amino-4-(2-fluoro-4-nitrophenoxy)pyridin-3-yl)propioloyl)piperazine-1-carboxylate

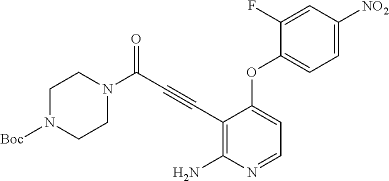 [SMILES: CC(C)(C)OC(=O)N1CCN(C(=O)C#Cc2c(Oc3ccc([N+](=O)[O-])cc3F)ccnc2N)CC1]

To a solution of 4-(2-fluoro-4-nitrophenoxy)-3-iodopyridin-2-amine (step D of intermediate 19, 1.00 g, 2.67 mmol), tert-butyl 4-propioloylpiperazine-1-carboxylate (intermediate 5, 953 mg, 4.00 mmol) and TEA (1.49 mL, 10.7 mmol) in DMF (9 mL) were added copper (I) iodide (102 mg, 0.53 mmol) and Pd(PPh₃)₄ (308 mg, 0.27 mmol) under N₂ at room temperature. The reaction mixture was subjected to microwave irradiation for 1 h at 90° C. The mixture was concentrated in vacuo, and the residue was purified by column chromatography on SiO₂ (EtOAc/MeOH=97/3) to afford the tert-butyl 4-(3-(2-amino-4-(2-fluoro-4-nitrophenoxy)pyridin-3-yl)propioloyl)piperazine-1-carboxylate (1.04 g, 80%) as a brown solid. ¹H-NMR (CDCl₃, Varian, 400 MHz): δ 1.47 (9H, s), 3.43 (4H, brs), 3.64 (2H brs), 3.73 (2H, brs), 5.35 (2H, brs), 6.10 (1H, d, J=6.0 Hz), 7.29 (1H, d, J=8.4 Hz), 8.03 (1H, d, J=6.0 Hz), 8.14 (2H, t, J=10.4 Hz).

### Step B: tert-Butyl 4-(3-(2-amino-4-(4-amino-2-fluorophenoxy)pyridin-3-yl)propioloyl)piperazine-1-carboxylate

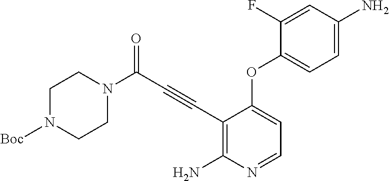 [SMILES: CC(C)(C)OC(=O)N1CCN(C(=O)C#Cc2c(Oc3ccc(N)cc3F)ccnc2N)CC1]

A mixture of tert-butyl 4-(3-(2-amino-4-(2-fluoro-4-nitrophenoxy)pyridin-3-yl)propioloyl)piperazine-1-carboxylate (1.04 g, 2.14 mmol), zinc (1.40 g, 21.4 mmol), and ammonium chloride (1.15 g, 21.4 mmol) in THF/MeOH (v/v=1/1, 22 mL) was stirred for 45 min at 60° C. The reaction mixture was filtered and the filtrate was partitioned between EtOAc and saturated NaHCO₃(aq.). The aqueous layer was extracted with EtOAc. The combined organic layer was washed with brine, dried over Na₂SO₄, and concentrated in vacuo. The residue was purified by column chromatography (EtOAc/MeOH=95/5) to afford the tert-butyl 4-(3-(2-amino-4-(4-amino-2-fluorophenoxy)pyridin-3-yl)propioloyl)piperazine-1-carboxylate (636 mg, 65%) as a yellow solid. ¹H-NMR (CDCl₃, Varian, 400 MHz): δ 1.46 (9H, s), 3.45 (4H, brs), 3.65 (2H brs), 3.81 (2H, brs), 3.85 (2H, brs), 5.22 (2H, brs), 5.98 (1H, d, J=6.0 Hz), 6.45 (1H, d, J=8.4 Hz), 6.51 (1H, dd, J=11.6, 2.4 Hz), 6.93 (1H, t, J=8.4 Hz), 7.90 (1H, d, J=6.0 Hz).

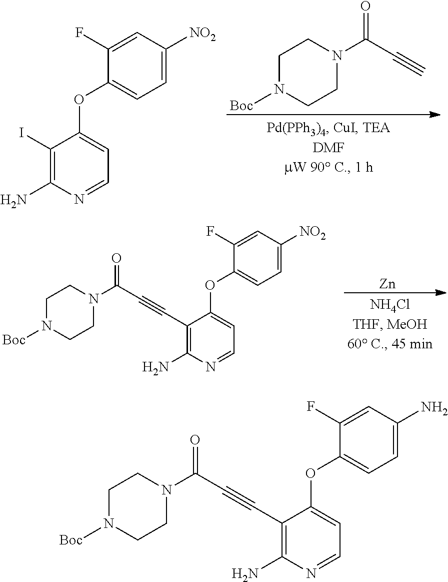

### Example 1

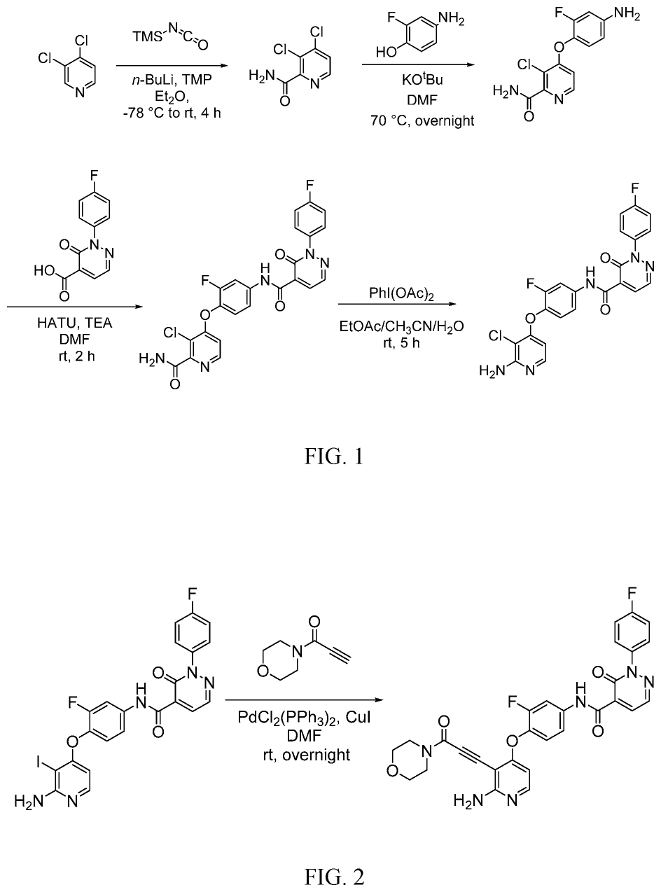

This example describes the synthesis of N-(4-(2-amino-3-chloropyridin-4-yloxy)-3-fluorophenyl)-2-(4-fluorophenyl)-3-oxo-2,3-dihydropyridazine-4-carboxamide in an aspect of the invention. See FIG. 1.

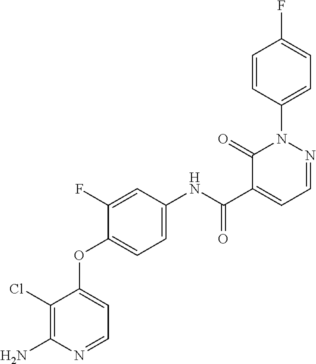 [SMILES: Nc1nccc(Oc2ccc(NC(=O)c3ccnn(-c4ccc(F)cc4)c3=O)cc2F)c1Cl]

### Step A: 3,4-Dichloropicolinamide

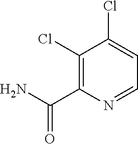 [SMILES: NC(=O)c1nccc(Cl)c1Cl]

To a solution of 2,2,6,6-tetramethylpiperidine (0.32 g, 2.23 mmol) in Et₂O (5 mL) was added n-BuLi (2 M in hexane, 0.89 mL, 2.23 mmol) via syringe over 15 min at 0° C. The resulting solution was stirred for 30 min at 0° C. and then cooled to −78° C. and stirring was continued for 30 min. To the mixture was slowly added a solution of 3,4-dichloropyridine (0.30 g, 2.03 mmol) in Et₂O (2 mL) via syringe for 15 min. The resulting mixture was stirred for 2 h at −78° C. and (trimethylsilyl)isocyanate (0.35 g, 3.04 mmol) was added to the mixture. After the addition, the cooling bath was removed and the reaction mixture was allowed to warm up to room temperature for 1 h. The reaction was quenched by acetic acid (4 mL) and water (10 mL). The mixture was allowed to stir overnight and the produced white solid was collected by filtration and washed with water/hexane to afford the 3,4-dichloropicolinamide (130 mg, 34%) as a white solid. ¹H-NMR (CDCl₃, Varian, 400 MHz): δ 5.66 (1H, brs), 7.49 (1H, brs), 7.58 (1H, d, J=5.2 Hz), 8.36 (1H, d, J=4.8 Hz)

### Step B: 4-(4-Amino-2-fluorophenoxy)-3-chloropicolinamide

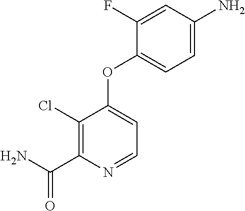 [SMILES: NC(=O)c1nccc(Oc2ccc(N)cc2F)c1Cl]

To a solution of 2-fluoro-4-nitrophenol (121 mg, 0.95 mmol) in DMF (10 mL) was added KO^{t}Bu (115 mg, 1.02 mmol). After being stirred at room temperature for 30 min, 4-(4-amino-2-fluorophenoxy)-3-chloropicolinamide (130 mg, 0.68 mmol) was added. The reaction mixture was heated overnight at 70° C. After cooling at room temperature, the mixture was diluted with EtOAc and water. The separated aqueous layer was extracted with EtOAc, the combined organic layer was dried over Na₂SO₄, filtered, and concentrated in vacuo. The residue was purified by column chromatography on SiO₂ (DCM/MeOH=50/1) to afford the 4-(4-amino-2-fluorophenoxy)-3-chloropicolinamide (51.0 mg, 27%) as a tan solid. ¹H-NMR (DMSO-d₆, Varian, 400 MHz): δ 5.52 (2H, s), 6.40-6.42 (1H, m), 6.48-6.52 (1H, m), 6.69 (1H, d, J=5.6 Hz), 7.00 (1H, t, J=8.8 Hz), 7.70 (1H, s), 8.00 (1H, s), 8.27 (1H, d, J=5.6 Hz).

### Step C: N-(4-(2-Carbamoyl-3-chloropyridin-4-yloxy)-3-fluorophenyl)-2-(4-fluorophenyl)-3-oxo-2,3-dihydropyridazine-4-carboxamide

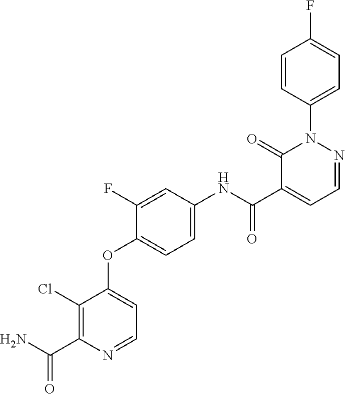 [SMILES: NC(=O)c1nccc(Oc2ccc(NC(=O)c3ccnn(-c4ccc(F)cc4)c3=O)cc2F)c1Cl]

A mixture of 4-(4-amino-2-fluorophenoxy)-3-chloropicolinamide (50.0 mg, 0.18 mmol), 2-(4-fluorophenyl)-3-oxo-2,3-dihydropyridazine-4-carboxylic acid (intermediate 15, 45.7 mg, 0.20 mmol), HATU (81.0 mg, 0.21 mmol), and TEA (0.03 mL, 0.21 mmol) in DMF (2 mL) was stirred for 2 h at room temperature. The mixture was diluted with EtOAc and water. The separated aqueous layer was extracted with EtOAc, the combined organic layer was dried over Na₂SO₄, filtered, and concentrated in vacuo to afford the N-(4-(2-carbamoyl-3-chloropyridin-4-yloxy)-3-fluorophenyl)-2-(4-fluorophenyl)-3-oxo-2,3-dihydropyridazine-4-carboxamide (70.0 mg, 79%) as a gray solid which was used to next step without further purification. ¹H-NMR (DMSO-d₆, Varian, 400 MHz): δ 5.95 (1H, d, J=5.2 Hz), 6.44 (2H, s), 7.33-7.43 (4H, m), 7.53 (1H, d, J=9.2 Hz), 7.66-7.69 (2H, m), 7.76 (1H, d, J=5.6 Hz), 7.98 (1H, d, J=10.0 Hz), 8.25 (1H, d, J=4.0 Hz), 8.37 (1H, d, J=4.0 Hz), 11.6 (1H, s).

### Step D: N-(4-(2-Amino-3-chloropyridin-4-yloxy)-3-fluorophenyl)-2-(4-fluorophenyl)-3-oxo-2,3-dihydropyridazine-4-carboxamide

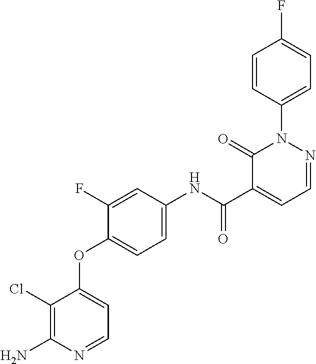 [SMILES: Nc1nccc(Oc2ccc(NC(=O)c3ccnn(-c4ccc(F)cc4)c3=O)cc2F)c1Cl]

A mixture of N-(4-(2-carbamoyl-3-chloropyridin-4-yloxy)-3-fluorophenyl)-2-(4-fluorophenyl)-3-oxo-2,3-dihydropyridazine-4-carboxamide (70.0 mg, 0.14 mmol) and PhI(OAc)₂ (67.9 mg, 0.21 mmol) in EtOAc/CH₃CN/H₂O (v/v/v=2/2/1, 5 mL) was stirred for 5 h at 0° C. The mixture was diluted with EtOAc and water. The separated aqueous layer was extracted with EtOAc, the combined organic layer was dried over Na₂SO₄, filtered, and concentrated in vacuo. The residue was purified by column chromatography on NH—SiO₂ (DCM/MeOH=50/1) and washed with Et₂O to afford the N-(4-(2-amino-3-chloropyridin-4-yloxy)-3-fluorophenyl)-2-(4-fluorophenyl)-3-oxo-2,3-dihydropyridazine-4-carboxamide (5.00 mg, 8%) as a pale yellow solid. ¹H-NMR (DMSO-d₆, Varian, 400 MHz): δ 5.95 (1H, d, J=5.2 Hz), 6.44 (2H, brs), 7.36-7.43 (3H, m), 7.52-7.56 (1H, m), 7.66-7.70 (2H, m), 7.76 (1H, d, J=5.6 Hz), 7.97-8.00 (1H, m), 8.25 (1H, d, J=3.6 Hz), 8.37 (1H, d, J=6.0 Hz), 11.66 (1H, s).

### Example 2

This example describes the synthesis of N-(4-(2-amino-3-(3-morpholino-3-oxoprop-1-ynyl)pyridin-4-yloxy)-3-fluorophenyl)-2-(4-fluorophenyl)-3-oxo-2,3-dihydropyridazine-4-carboxamide in an aspect of the invention. See FIG. 2.

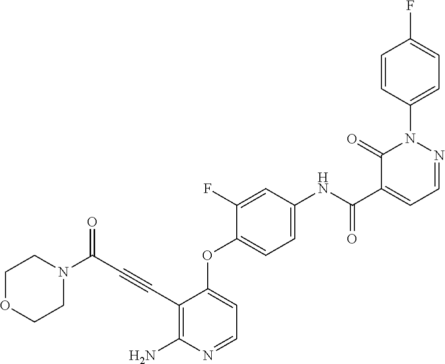 [SMILES: Nc1nccc(Oc2ccc(NC(=O)c3ccnn(-c4ccc(F)cc4)c3=O)cc2F)c1C#CC(=O)N1CCOCC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 150 mg, 0.27 mmol), 1-morpholinoprop-2-yn-1-one (intermediate 1, 74.4 mg, 0.53 mmol), PdCl₂(PPh₃)₂ (18.8 mg, 0.03 mmol), copper(I) iodide (5.09 mg, 0.03 mmol), and TEA (0.07 mL, 0.53 mmol) in DMF (5 mL) was stirred overnight at 80° C. The mixture was filtered through CELITE™, the solvent was removed by evaporation. The residue was dissolved in CH₃CN. After stirring for 3 h at room temperature, the precipitated solid was collected by filtration to afford the N-(4-(2-amino-3-(3-morpholino-3-oxoprop-1-ynyl)pyridin-4-yloxy)-3-fluorophenyl)-2-(4-fluorophenyl)-3-oxo-2,3-dihydropyridazine-4-carboxamide (25.0 mg, 16%) as a yellow solid. ¹H-NMR (DMSO-d₆, Varian, 400 MHz): δ 3.48-3.58 (6H, m), 3.70-3.72 (2H, m), 5.88 (1H, d, J=5.2 Hz), 6.57 (2H, brs), 7.35-7.40 (3H, m), 7.49-7.51 (1H, m), 7.63-7.66 (2H, m), 7.94-7.97 (1H, m), 8.22 (1H, d, J=4.4 Hz), 8.34 (1H, d, J=4.0 Hz), 11.63 (1H, s). * NH peak was not observed.

### Example 3

This example describes the synthesis of N-(4-(2-amino-3-(morpholinoprop-1-ynyl)pyridine-4-yloxy)-3-fluorophenyl)-2-(4-fluorophenyl)-3-oxo-2,3-dihydropyridazine-4-carboxamide in an aspect of the invention.

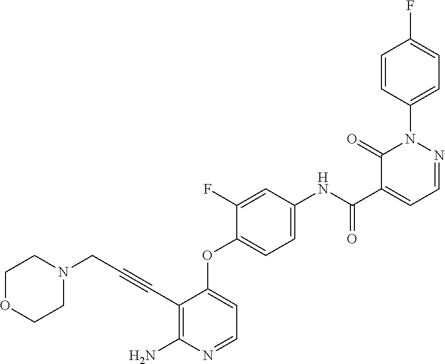 [SMILES: Nc1nccc(Oc2ccc(NC(=O)c3ccnn(-c4ccc(F)cc4)c3=O)cc2F)c1C#CCN1CCOCC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 130 mg, 0.23 mmol), 4-(prop-2-ynyl)morpholine (intermediate 2, 58.0 mg, 0.46 mmol), PdCl₂(PPh₃)₂ (16.3 mg, 0.02 mmol) and copper (I) iodide (4.41 mg, 0.02 mmol) in THF/TEA (v/v=1/1, 20 mL) was heated to reflux for 5 h at 90° C. The mixture was filtered through a CELITE™ pad, and the filtrate was concentrated in vacuo. The residue was purified by column chromatography on SiO₂ (EtOAc/MeOH=30/1 to EtOAc) to afford the N-(4-(2-amino-3-(morpholinoprop-1-ynyl)pyridine-4-yloxy)-3-fluorophenyl)-2-(4-fluorophenyl)-3-oxo-2,3-dihydropyridazine-4-carboxamide (60.0 mg, 46%) as a pale yellow solid. ¹H-NMR (DMSO-d₆, Varian, 400 MHz): δ 2.43-2.52 (4H, m), 3.52-3.62 (6H, m), 5.93 (1H, d, J=6.0 Hz), 6.26 (2H, s), 7.27 (1H, t, J=8.8 Hz), 7.34-7.46 (2H, m), 7.47-7.54 (1H, m), 7.63-7.72 (2H, m), 7.80 (1H, d, J=6.0 Hz), 7.92-8.00 (1H, m), 8.25 (1H, d, J=4.0 Hz), 8.37 (1H, d, J=3.6 Hz), 11.64 (1H, s)

### Example 4

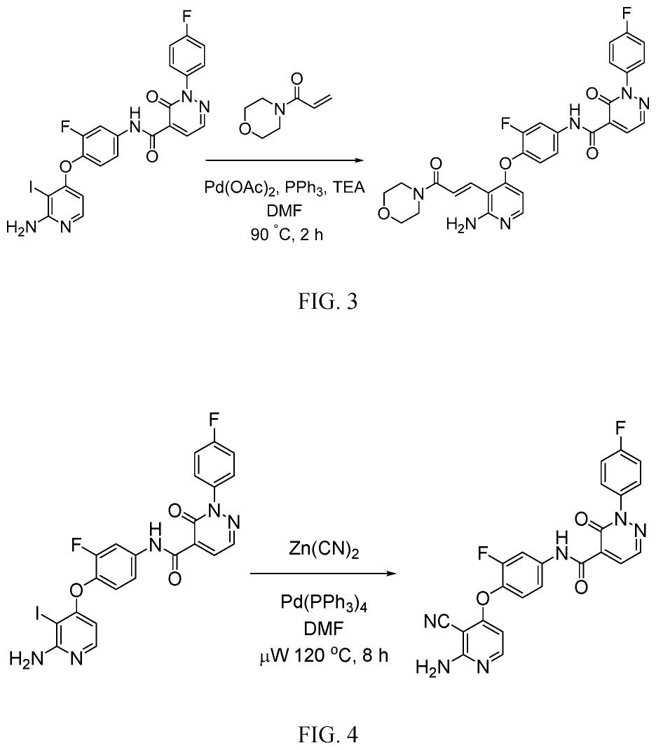

This example describes the synthesis of (E)-N-(4-(2-amino-3-(3-morpholino-3-oxoprop-1-enyl)pyridine-4-yloxy)-3-fluorophenyl)-2-(4-fluorophenyl)-3-oxo-2,3-dihydropyridazine-4-carboxamide in an aspect of the invention. See FIG. 3.

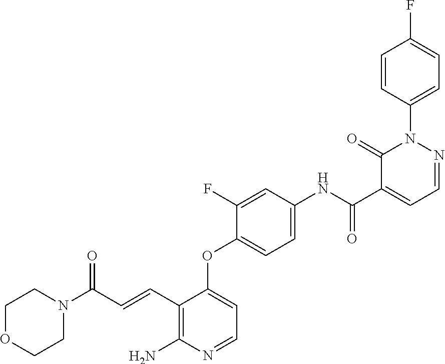 [SMILES: Nc1nccc(Oc2ccc(NC(=O)c3ccnn(-c4ccc(F)cc4)c3=O)cc2F)c1/C=C/C(=O)N1CCOCC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 100 mg, 0.18 mmol), 1-morpholinoprop-2-en-1-one (intermediate 3, 38.0 mg, 0.27 mmol), PPh₃ (4.67 mg, 0.02 mmol) and TEA (36.0 μL, 0.36 mmol) in DMF (5 mL) was stirred for 2 h at 90° C. The mixture was filtered through a CELITE™ pad, and the filtrate was concentrated in vacuo. The residue was purified by column chromatography on SiO₂ (EtOAc/MeOH=50/1) to afford the (E)-N-(4-(2-amino-3-(3-morpholino-3-oxoprop-1-enyl)pyridine-4-yloxy)-3-fluorophenyl)-2-(4-fluorophenyl)-3-oxo-2,3-dihydropyridazine-4-carboxamide (100 mg, 98%) as a pale-yellow solid. ¹H-NMR (CDCl₃, Varian, 400 MHz): δ 3.52-3.81 (8H, m), 4.89 (2H, s), 6.04 (1H, d, J=6.0 Hz), 7.06-7.13 (1H, m), 7.15 (1H, d, J=15.6 Hz), 7.19-7.30 (2H, m), 7.31-7.38 (1H, m), 7.55-7.64 (2H, m), 7.72 (1H, d, J=15.6 Hz), 7.86 (1H, d, J=5.6 Hz), 7.88-7.96 (1H, m), 8.24 (1H, d, J=4.0 Hz), 8.41 (1H, d, J=4.4 Hz), 11.80 (1H, s).

### Example 5

This example describes the synthesis of N-(4-(2-amino-3-(3-cyanopyridin-4-yloxy)-3-fluorophenyl)-2-(4-fluorophenyl)-3-oxo-2,3-dihydropyridazine-4-carboxamide in an aspect of the invention. See FIG. 4.

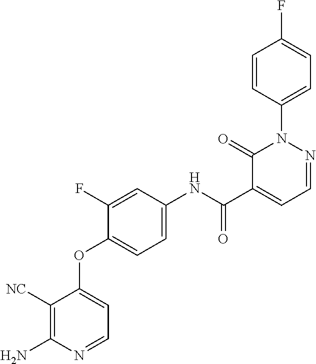 [SMILES: N#Cc1c(Oc2ccc(NC(=O)c3ccnn(-c4ccc(F)cc4)c3=O)cc2F)ccnc1N]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 90.0 mg, 0.16 mmol), Pd(PPh₃)₄ (18.5 mg, 0.02 mmol), and Zn(CN)₂ (37.7 mg, 0.32 mmol) in DMF (5 mL) was sealed and the reaction mixture was submitted to microwave irradiation for 8 h at 120° C. The mixture was filtered through a CELITE™ pad, and the filtrate was concentrated in vacuo. The residue was purified by column chromatography on SiO₂ (EtOAc/MeOH=50/1), and triturated in Et₂O to afford the N-(4-(2-amino-3-(3-cyanopyridin-4-yloxy)-3-fluorophenyl)-2-(4-fluorophenyl)-3-oxo-2,3-dihydropyridazine-4-carboxamide (50.0 mg, 68%) as a pale yellow solid. ¹H-NMR (DMSO-d₆, Varian, 400 MHz): δ 5.87 (1H, d, J=5.6 Hz), 7.06 (2H, brs), 7.32-7.48 (3H, m), 7.50-7.57 (1H, m), 7.60-7.69 (2H, m), 7.94-8.04 (2H, m), 8.21 (1H, d, J=4.0 Hz), 8.33 (1H, d, J=4.0 Hz), 11.64 (1H, s).

### Example 6

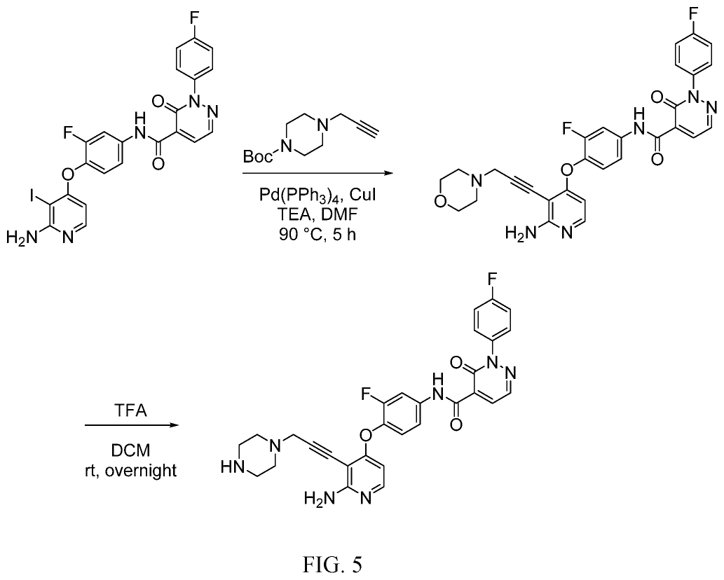

This example describes the synthesis of N-(4-(2-amino-3-(3-(piperazin-1-yl)prop-1-ynyl)pyridin-4-yloxy)-3-fluorophenyl)-2-(4-fluorophenyl)-3-oxo-2,3-dihydropyridazine-4-carboxamide in an aspect of the invention. See FIG. 5.

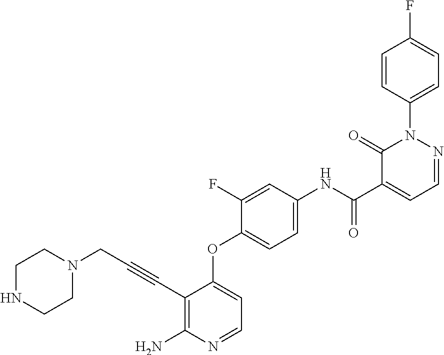 [SMILES: Nc1nccc(Oc2ccc(NC(=O)c3ccnn(-c4ccc(F)cc4)c3=O)cc2F)c1C#CCN1CCNCC1]

### Step A: tert-Butyl 4-(3-(2-amino-4-(2-fluoro-4-(2-(4-fluorophenyl)-3-oxo-2,3-dihydropyridazine-4-carboxamido)phenoxy)pyridin-3-yl)prop-2-ynyl)piperazine-1-carboxylate

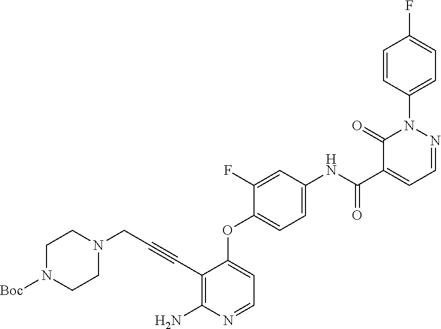 [SMILES: CC(C)(C)OC(=O)N1CCN(CC#Cc2c(Oc3ccc(NC(=O)c4ccnn(-c5ccc(F)cc5)c4=O)cc3F)ccnc2N)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 2.0 g, 3.56 mmol), tert-butyl 4-(prop-2-ynyl)piperazine-1-carboxylate (intermediate 4, 1.20 g, 5.34 mmol), Pd(PPh₃)₄ (0.41 g, 0.36 mmol), copper(I) iodide (0.13 g, 0.71 mmol), and TEA (2.0 mL, 14.25 mmol) in DMF (15 mL) was stirred for 5 h at 90° C. After being cooled at room temperature, the reaction mixture was dissolved in EtOAc and washed with saturated NH₄Cl (aq.). The organic layer was dried over Na₂SO₄, filtered and concentrated in vacuo. The residue was purified by column chromatography on SiO₂ (EtOAc/MeOH=95/5) to afford the tert-butyl 4-(3-(2-amino-4-(2-fluoro-4-(2-(4-fluorophenyl)-3-oxo-2,3-dihydropyridazine-4-carboxamido)phenoxy)pyridin-3-yl)prop-2-ynyl)piperazine-1-carboxylate (1.68 g, 71%) as a brown solid. ¹H-NMR (CDCl₃, Varian, 400 MHz): δ 1.45 (9H, s), 2.58 (4H, brs), 3.48 (4H, brs), 3.63 (2H, s), 5.07 (2H, brs), 5.99 (1H, d, J=5.6 Hz), 7.14 (1H, t, J=8.8 Hz), 7.23-7.26 (2H, m), 7.37 (1H, d, J=8.8 Hz), 7.58-7.61 (2H, m), 7.83 (1H, d, J=6.0 Hz), 7.90 (1H, d, J=11.6 Hz), 8.23 (1H, d, J=4.4 Hz), 8.40 (1H, d, J=4.4 Hz), 11.78 (1H, brs).

### Step B: N-(4-(2-Amino-3-(3-(piperazin-1-yl)prop-1-ynyl)pyridin-4-yloxy)-3-fluorophenyl)-2-(4-fluorophenyl)-3-oxo-2,3-dihydropyridazine-4-carboxamide

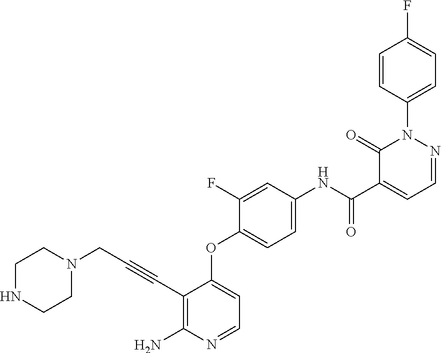 [SMILES: Nc1nccc(Oc2ccc(NC(=O)c3ccnn(-c4ccc(F)cc4)c3=O)cc2F)c1C#CCN1CCNCC1]

To a solution of tert-butyl 4-(3-(2-amino-4-(2-fluoro-4-(2-(4-fluorophenyl)-3-oxo-2,3-dihydropyridazine-4-carboxamido)phenoxy)pyridin-3-yl)prop-2-ynyl)piperazine-1-carboxylate (156 mg, 0.24 mmol) in DCM (2 mL) was added TFA (731 μL, 9.49 mmol) at room temperature. The reaction mixture was stirred overnight at room temperature. The excess TFA and solvent was removed by evaporation, and the residue was basified with saturated NaHCO₃(aq.) and extracted with EtOAc. The combined organic layer was washed with brine, dried over Na₂SO₄, filtered, and concentrated in vacuo. The residue was purified by column chromatography on NH—SiO₂ (DCM/MeOH=97/3) to afford the N-(4-(2-amino-3-(3-(piperazin-1-yl)prop-1-ynyl)pyridin-4-yloxy)-3-fluorophenyl)-2-(4-fluorophenyl)-3-oxo-2,3-dihydropyridazine-4-carboxamide (802 mg, 61%) as a yellow solid. ¹H-NMR (CDCl₃, Varian, 400 MHz): δ 2.59 (4H, brs), 2.93 (4H, t, J=4.4 Hz), 3.60 (2H, s), 5.06 (2H, brs), 6.00 (1H, d, J=6.0 Hz), 7.13 (1H, t, J=8.8 Hz), 7.22-7.26 (2H, m), 7.33 (1H, d, J=8.8 Hz), 7.58-7.61 (2H, m), 7.84 (1H, d, J=6.0 Hz), 7.90 (1H, d, J=11.6 Hz), 8.23 (1H, d, J=4.4 Hz), 8.40 (1H, d, J=4.4 Hz), 11.78 (1H, brs). *NH peak was not observed.

### Example 7

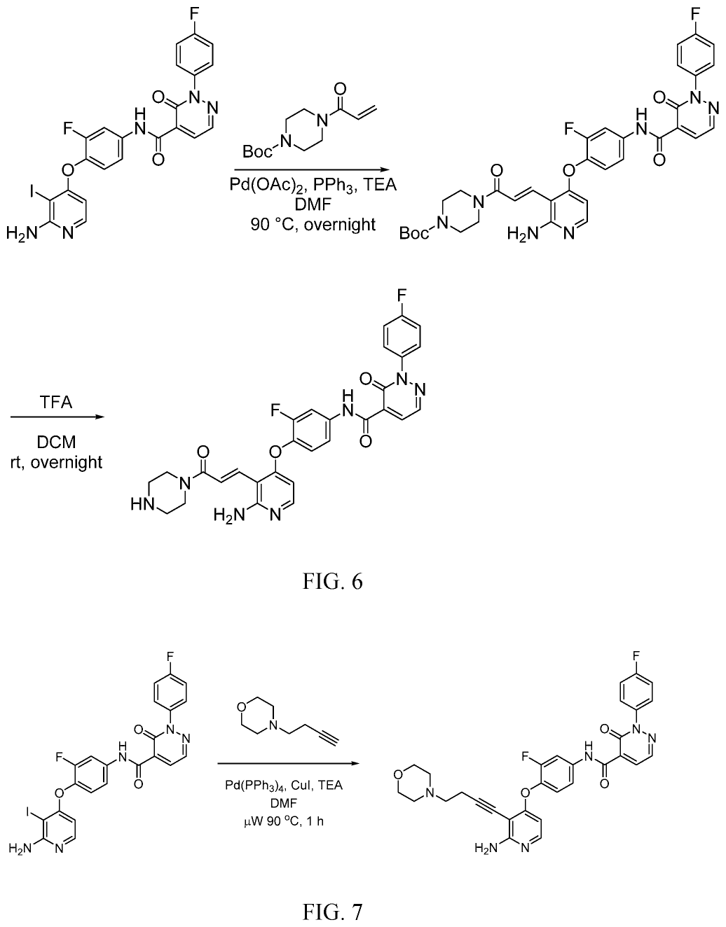

This example describes the synthesis of (E)-N-(4-(2-amino-3-(3-oxo-3-(piperazin-1-yl)prop-1-enyl)pyridin-4-yloxy)-3-fluorophenyl)-2-(4-fluorophenyl)-3-oxo-2,3-dihydropyridazine-4-carboxamide in an aspect of the invention. See FIG. 6.

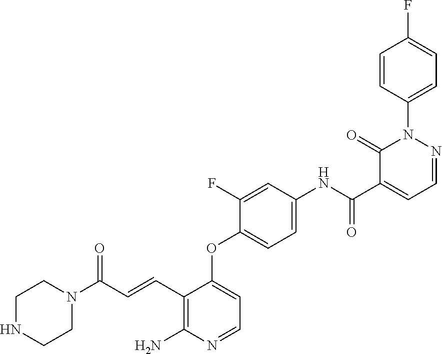 [SMILES: Nc1nccc(Oc2ccc(NC(=O)c3ccnn(-c4ccc(F)cc4)c3=O)cc2F)c1/C=C/C(=O)N1CCNCC1]

### Step A: (E)-tert-butyl 4-(3-(2-amino-4-(2-fluoro-4-(2-(4-fluorophenyl)-3-oxo-2,3-dihydropyridazine-4-carboxamido)phenoxy)pyridin-3-yl)acryloyl)piperazine-1-carboxylate

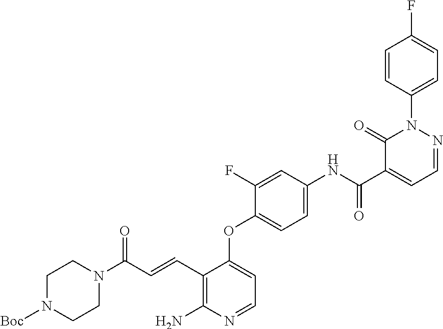 [SMILES: CC(C)(C)OC(=O)N1CCN(C(=O)/C=C/c2c(Oc3ccc(NC(=O)c4ccnn(-c5ccc(F)cc5)c4=O)cc3F)ccnc2N)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 200 mg, 0.36 mmol), tert-butyl 4-acryloykpiperazine-1-carboxylate (intermediate 6, 128 mg, 0.53 mmol), PPh₃ (9.35 mg, 0.04 mmol), TEA (99 μL, 0.71 mmol), and Pd(OAc)₂ (4.00 mg, 0.02 mmol) in DMF (3.6 mL) was stirred overnight at 90° C. After being cooled to room temperature, the reaction mixture was concentrated in vacuo. The residue was diluted EtOAc, washed with water and brine, dried over Na₂SO₄, filtered, and concentrated in vacuo. The residue was purified by column chromatography (EtOAc/MeOH=97/3 to 95/5) to afford the (E)-tert-butyl 4-(3-(2-amino-4-(2-fluoro-4-(2-(4-fluorophenyl)-3-oxo-2,3-dihydropyridazine-4-carboxamido)phenoxy)pyridin-3-yl)acryloyl)piperazine-1-carboxylate (208 mg, 87%) as a brown solid. ¹H-NMR (DMSO-d₆, Varian, 400 MHz): δ 1.41 (9H, s), 3.33 (4H, brs), 3.55 (4H brs), 5.90 (1H, d, J=5.2 Hz), 6.35 (2H, brs), 7.11 (1H, d, J=15.6 Hz), 7.35 (1H, t, J=8.8 Hz), 7.41 (2H, t, J=8.8 Hz), 7.53 (1H, d, J=9.6 Hz), 7.64 (1H, d, J=15.6 Hz), 7.66-7.70 (2H, m), 7.80 (1H, d, J=5.6 Hz), 7.98 (1H, dd, J=12.8, 1.6 Hz), 8.26 (1H, d, J=4.0 Hz), 8.37 (1H, d, J=4.4 Hz), 11.66 (1H, brs).

### Step B: (E)-N-(4-(2-amino-3-(3-oxo-3-(piperazin-1-yl)prop-1-enyl)pyridin-4-yloxy)-3-fluorophenyl)-2-(4-fluorophenyl)-3-oxo-2,3-dihydropyridazine-4-carboxamide

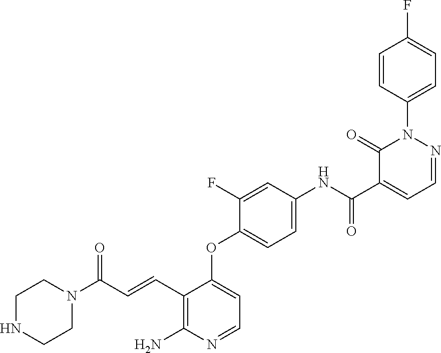 [SMILES: Nc1nccc(Oc2ccc(NC(=O)c3ccnn(-c4ccc(F)cc4)c3=O)cc2F)c1/C=C/C(=O)N1CCNCC1]

To a solution of (E)-tert-butyl 4-(3-(2-amino-4-(2-fluoro-4-(2-(4-fluorophenyl)-3-oxo-2,3-dihydropyridazine-4-carboxamido)phenoxy)pyridin-3-yl)acryloyl)piperazine-1-carboxylate (208 mg, 0.329 mmol) in DMF (2.2 ml) was added TFA (952 μL, 12.4 mmol) at room temperature. The reaction mixture was stirred overnight at room temperature. The reaction mixture was concentrated in vacuo. The residue was diluted with DCM, and then neutralized with TEA. The mixture was stirred at room temperature for 10 min and concentrated in vacuo. The residue was purified by column chromatography on NH—SiO₂ (EtOAc:MeOH=95:5) to afford the (E)-N-(4-(2-amino-3-(3-oxo-3-(piperazin-1-yl)prop-1-enyl)pyridin-4-yloxy)-3-fluorophenyl)-2-(4-fluorophenyl)-3-oxo-2,3-dihydropyridazine-4-carboxamide (132 mg, 74%) as a yellow solid. ¹H-NMR (DMSO-d₆, Varian, 400 MHz): δ 2.66 (4H, brs), 3.47 (4H brs), 5.90 (1H, d, J=5.2 Hz), 6.32 (2H, brs), 7.11 (1H, d, J=15.6 Hz), 7.35 (1H, t, J=8.8 Hz), 7.41 (2H, t, J=8.8 Hz), 7.52 (1H, d, J=8.8 Hz), 7.59 (1H, d, J=16.0 Hz), 7.66-7.70 (2H, m), 7.80 (1H, d, J=5.6 Hz), 7.98 (1H, d, J=12.8 Hz), 8.25 (1H, d, J=3.6 Hz), 8.37 (1H, d, J=4.4 Hz), 11.66 (1H, brs). *NH peak was not observed.

### Example 8

This example describes the synthesis of N-(4-(2-amino-3-(4-morpholinobut-1-ynyl)pyridin-4-yloxy)-3-fluorophenyl)-2-(4-fluorophenyl)-3-oxo-2,3-dihydropyridazine-4-carboxamide in an aspect of the invention. See FIG. 7.

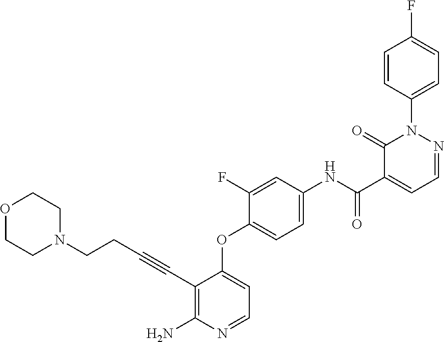 [SMILES: Nc1nccc(Oc2ccc(NC(=O)c3ccnn(-c4ccc(F)cc4)c3=O)cc2F)c1C#CCCN1CCOCC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 100 mg, 0.18 mmol), 4-(but-3-ynyl)morpholine (intermediate 9, 74.4 mg, 0.53 mmol), Pd(PPh₃)₄ (41.2 mg, 0.04 mmol), and copper (I) iodide (3.39 mg, 0.02 mmol) in DMF/TEA (v/v=1/0.15, 1.15 mL) was purged with N₂. The reaction mixture was subjected to microwave irradiation for 1 h at 90° C. After cooled to room temperature, the reaction mixture was filtered through a CELITE™ pad, and the filtrate was concentrated in vacuo. EtOAc and water were poured into the residue, and the separated aqueous layer was extracted with EtOAc. The combined organic layer was dried over Na₂SO₄, filtered, and concentrated in vacuo. The residue was purified by column chromatography (EtOAc/MeOH=5/1) to afford the N-(4-(2-amino-3-(4-morpholinobut-1-ynyl)pyridin-4-yloxy)-3-fluorophenyl)-2-(4-fluorophenyl)-3-oxo-2,3-dihydropyridazine-4-carboxamide (94.0 mg, 92%) as a pale yellow solid. ¹H-NMR (CDCl₃, Varian, 400 MHz): δ 2.54 (4H, m), 2.65-2.70 (4H, m), 3.71 (4H, m), 5.41 (2H, s), 5.94 (1H, d, J=5.2 Hz), 7.14 (1H, t, J=9.6 Hz), 7.23-7.26 (2H, m), 7.32-7.34 (1H, m), 7.57-7.61 (2H, m), 7.81 (1H, d, J=6.0 Hz), 7.87-7.91 (1H, m), 8.23 (1H, d, J=4.0 Hz), 8.40 (1H, d, J=4.0 Hz), 11.78 (1H, brs).

### Example 9

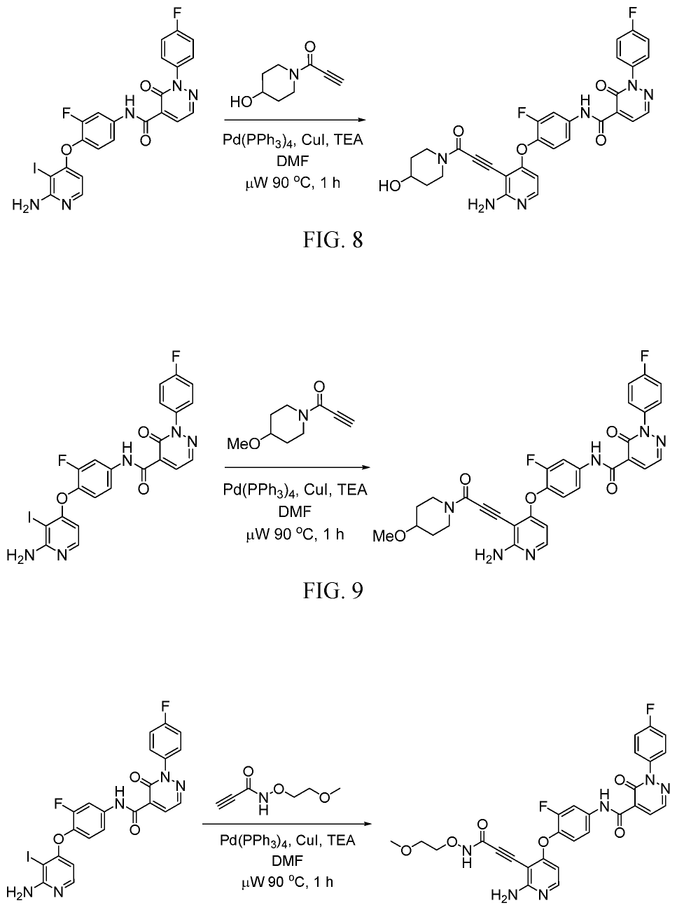

This example describes the synthesis of N-(4-(2-amino-3-(3-(4-hydroxypiperidin-1-yl)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention. See FIG. 8.

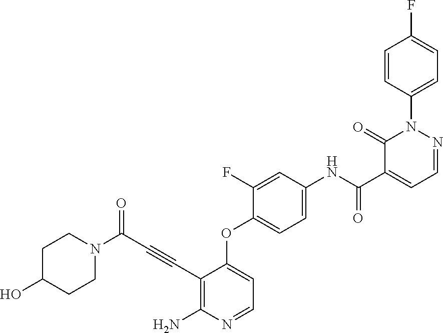 [SMILES: Nc1nccc(Oc2ccc(NC(=O)c3ccnn(-c4ccc(F)cc4)c3=O)cc2F)c1C#CC(=O)N1CCC(O)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 100 mg, 0.18 mmol), 1-(4-hydroxypipericin-1-yl)prop-2-yn-1-one (intermediate 7, 40.9 mg, 0.27 mmol), Pd(PPh₃)₄ (41.2 mg, 0.04 mmol), and copper (I) iodide (3.39 mg, 0.02 mmol) in DMF/TEA (v/v=1/0.15, 1.15 mL) was purged with N₂. The reaction mixture was subjected to microwave irradiation for 1 h at 90° C. After cooled to room temperature, the reaction mixture was filtered through a CELITE™ pad, and the filtrate was concentrated in vacuo. EtOAc and water were poured into the residue, and the separated aqueous layer was extracted with EtOAc. The combined organic layer was dried over Na₂SO₄, filtered, and concentrated in vacuo. The residue was purified by column chromatography (EtOAc/MeOH=10/1) to afford the N-(4-(2-amino-3-(3-(4-hydroxypiperidin-1-yl)-3-oxoprop-1-ynyl)pyridin-4-yloxy)-3-fluorophenyl)-2-(4-fluorophenyl)-3-oxo-2,3-dihydropyridazine-4-carboxamide (65.2 mg, 62%) as a pale yellow solid. ¹H-NMR (CDCl₃, Varian, 400 MHz): δ 1.68 (3H, m), 1.89 (2H, m), 3.36 (1H, m), 3.58 (1H, m), 3.97 (2H, m), 4.07 (1H, m), 4.16 (1H, m), 5.28 (2H, s), 6.00 (1H, m), 7.15 (1H, t, J=7.6 Hz), 7.26-7.28 (1H, m), 7.34-7.36 (1H, m), 7.59-7.61 (2H, m), 7.91-7.94 (2H, m), 8.25 (1H, m), 8.41 (1H, m), 11.82 (1H, brs).

### Example 10

This example describes the synthesis of N-(4-(2-amino-3-(3-(4-methoxypiperidin-1-yl)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention. See FIG. 9.

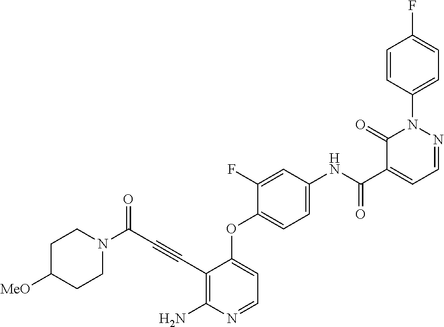 [SMILES: COC1CCN(C(=O)C#Cc2c(Oc3ccc(NC(=O)c4ccnn(-c5ccc(F)cc5)c4=O)cc3F)ccnc2N)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 90.0 mg, 0.16 mmol), 1-(4-methoxypiperidin-1-yl)prop-2-yn-1-one (intermediate 8, 40.2 mg, 0.24 mmol), Pd(PPh₃)₄ (37.1 mg, 0.03 mmol) and copper (I) iodide (3.05 mg, 0.02 mmol) in DMF/TEA (v/v=1/0.15, 3.30 mL) was purged with N₂. The reaction mixture was subjected to microwave irradiation for 1 h at 90° C. After cooled to room temperature, the reaction mixture was filtered through a, and the CELITE™ pad, and the filtrate was concentrated in vacuo. EtOAc and water were poured into the residue, and the separated aqueous layer was extracted with EtOAc. The combined organic layer was dried over Na₂SO₄, filtered, and concentrated in vacuo. The residue was purified by column chromatography (EtOAc) to afford the N-(4-(2-amino-3-(3-(4-methoxypiperidin-1-yl)-3-oxoprop-1-ynyl)pyri din-4-yloxy)-3-fluorophenyl)-2-(4-fluorophenyl)-3-oxo-2,3-dihydropyridazine-4-carboxamide (33.8 mg, 35%) as a pale yellow solid. ¹H-NMR (CDCl₃, Varian, 400 MHz): δ 1.62 (2H, m), 1.84 (2H, m), 3.34 (3H, s), 3.46 (1H, m), 3.67 (1H, m), 3.84 (1H, m), 4.04 (1H, m), 5.23 (2H, s), 6.44 (1H, d, J=4.0 Hz), 7.15 (1H, t, J=8.0 Hz), 7.23-7.26 (2H, m), 7.34 (2H, m), 7.53-7.58 (2H, m), 7.91-7.92 (2H, m), 8.24 (1H, d, J=4.0 Hz), 8.41 (1H, d, J=4.0 Hz), 11.76 (1H, brs).

### Example 11

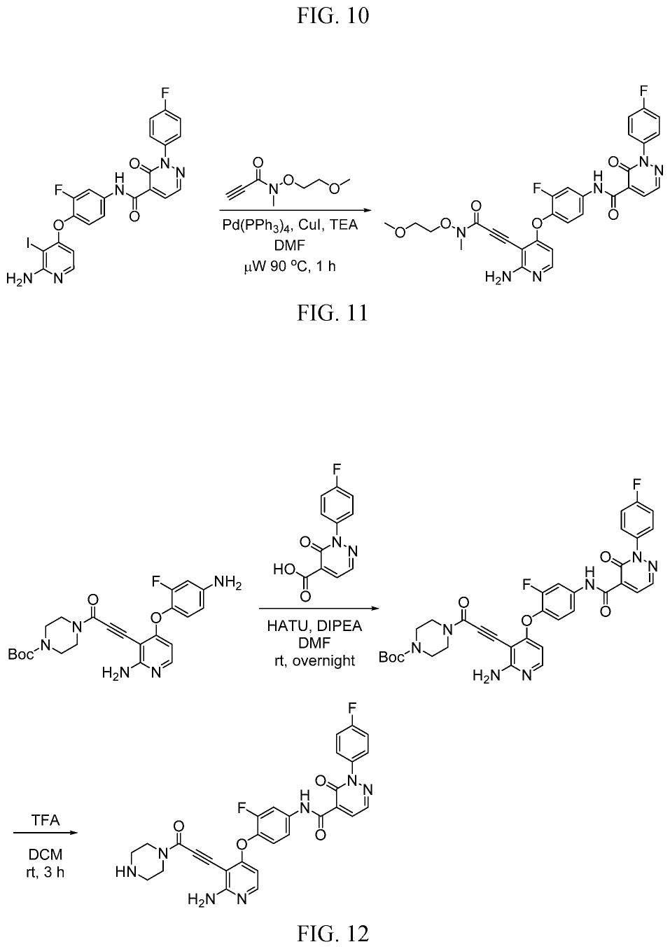

This example describes the synthesis of N-(4-(2-amino-3-(3-(2-methoxyethoxyamino)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention. See FIG. 10.

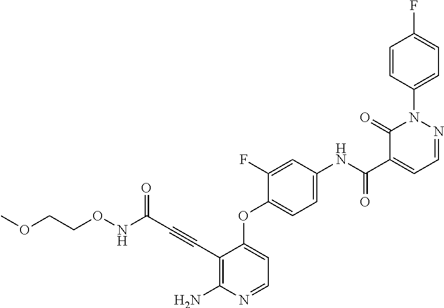 [SMILES: COCCONC(=O)C#Cc1c(Oc2ccc(NC(=O)c3ccnn(-c4ccc(F)cc4)c3=O)cc2F)ccnc1N]

To a solution of N-(4-(2-amino-3-iodopyridin-4-yloxy)-3-fluorophenyl)-2-(4-fluorophenyl)-3-oxo-2,3-dihydropyridazine-4-carboxamide (intermediate 19, 200 mg, 0.36 mmol), N-(2-methoxyethoxy)propiolamide (intermediate 10, 102 mg, 0.71 mmol) and TEA (199 μL, 1.42 mmol) in DMF (1.8 ml) were added copper (I) iodide (14.0 mg, 0.071 mmol) and Pd(PPh₃)₄ (41.0 mg, 0.036 mmol) under N₂ at room temperature. The reaction mixture was subjected to microwave irradiation for 1 h at 90° C. The mixture was concentrated in vacuo, and the residue was purified by column chromatography (EtOAc/MeOH=95/5) to afford the N-(4-(2-amino-3-(3-(2-methoxyethoxyamino)-3-oxoprop-1-ynyl)pyridin-4-yloxy)-3-fluorophenyl)-2-(4-fluorophenyl)-3-oxo-2,3-dihydropyridazine-4-carboxamide (17.1 mg, 13%) as a brown solid. ¹H-NMR (CDCl₃, Varian, 400 MHz): δ 3.22 (3H, s), 3.60-3.69 (2H, m), 4.10-4.15 (2H, m), 5.39 (2H, brs), 5.92 (1H, d, J=5.6 Hz), 7.15 (1H, brs), 7.23-7.26 (4H, m), 7.35 (1H, d, J=8.0 Hz), 7.55-7.61 (2H, m), 7.92 (1H, d, J=10.0 Hz), 8.23 (1H, d, J=4.0 Hz), 8.40 (1H, d, J=4.0 Hz), 11.82 (1H, brs).

### Example 12

This example describes the synthesis of N-(4-(2-amino-3-(3-((2-methoxyethoxy)(methyl)amino)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention. See FIG. 11.

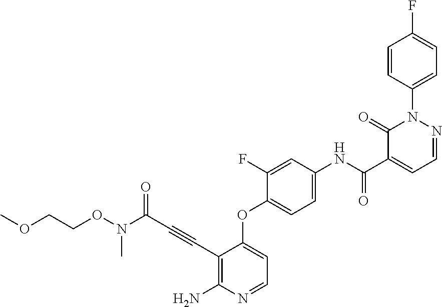 [SMILES: COCCON(C)C(=O)C#Cc1c(Oc2ccc(NC(=O)c3ccnn(-c4ccc(F)cc4)c3=O)cc2F)ccnc1N]

To a solution of N-(4-(2-amino-3-iodopyridin-4-yloxy)-3-fluorophenyl)-2-(4-fluorophenyl)-3-oxo-2,3-dihydropyridazine-4-carboxamide (intermediate 19, 300 mg, 0.53 mmol), N-(2-methoxyethoxy)-N-methylpropiolamide (intermediate 11, 153 mg, 0.96 mmol) and TEA (298 μL, 2.14 mmol) in DMF (2.7 mL) were added copper (I) iodide (20.0 mg, 0.11 mmol) and Pd(PPh₃)₄ (62.0 mg, 0.053 mmol) under N₂ at room temperature. The reaction mixture was subjected to microwave irradiation for 1 h at 90° C. The mixture was concentrated in vacuo, and the residue was purified by column chromatography (EtOAc/MeOH=95/5) to afford the N-(4-(2-amino-3-(3-((2-methoxyethoxy)(methyl)amino)-3-oxoprop-1-ynyl)pyridin-4-yloxy)-3-fluorophenyl)-2-(4-fluorophenyl)-3-oxo-2,3-dihydropyridazine-4-carboxamide (95.2 mg, 30%) as a brown solid. ¹H-NMR (CDCl₃, Varian, 400 MHz): δ 3.30 (3H, s), 3.43 (3H, s), 3.55-3.64 (2H, m), 4.31-4.42 (2H, m), 5.33 (2H, brs), 5.93 (1H, d, J=6.0 Hz), 7.23-7.26 (3H, m), 7.36 (1H, d, J=8.8 Hz), 7.58-7.61 (2H, m), 7.90-7.95 (2H, m), 8.24 (1H, d, J=4.4 Hz), 8.40 (1H, d, J=4.4 Hz), 11.82 (1H, brs).

### Example 13

This example describes the synthesis of N-(4-(2-amino-3-(3-oxo-3-(piperazin-1-yl)prop-1-ynyl)pyridin-4-yloxy)-3-fluorophenyl)-2-(4-fluorophenyl)-3-oxo-2,3-dihydropyridazine-4-carboxamide in an aspect of the invention. See FIG. 12.

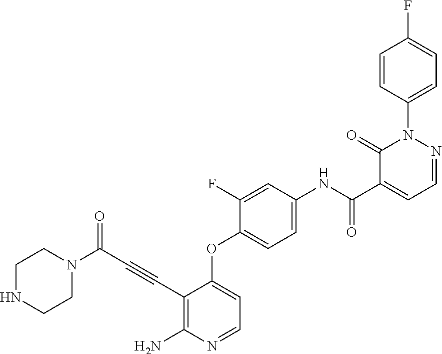 [SMILES: Nc1nccc(Oc2ccc(NC(=O)c3ccnn(-c4ccc(F)cc4)c3=O)cc2F)c1C#CC(=O)N1CCNCC1]

### Step A: tert-Butyl 4-(3-(2-amino-4-(2-fluoro-4-(2-(4-fluorophenyl)-3-oxo-2,3-dihydropyridazine-4-carboxamido)phenoxy)pyridine-3-yl)piperazine-1-carboxylate

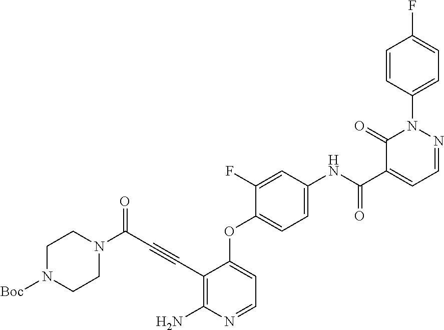 [SMILES: CC(C)(C)OC(=O)N1CCN(C(=O)C#Cc2c(Oc3ccc(NC(=O)c4ccnn(-c5ccc(F)cc5)c4=O)cc3F)ccnc2N)CC1]

A mixture of tert-butyl 4-(3-(2-amino-4-(4-amino-2-fluorophenoxy)pyridin-3-yl)propioloyl)piperazine-1-carboxylate (intermediate 20, 636 mg, 1.40 mmol), 2-(4-fluorophenyl)-3-oxo-2,3-dihydropyridazine-4-carboxylic acid (intermediate 15, 654 mg, 2.79 mmol), HATU (797 mg, 2.09 mmol) and DIPEA (976 μL, 5.59 mmol) in DMF (14 mL) was stirred overnight at room temperature. The reaction mixture was partitioned between water and EtOAc. The aqueous layer was extracted with EtOAc. The combined organic layer was washed with water and brine, dried over Na₂SO₄, filtered, and concentrated in vacuo. The residue was purified by column chromatography (EtOAc/MeOH=97/3 to 95/5) to afford the tert-butyl 4-(3-(2-amino-4-(2-fluoro-4-(2-(4-fluorophenyl)-3-oxo-2,3-dihydropyridazine-4-carboxamido)phenoxy)pyridine-3-yl)piperazine-1-carboxylate (476 mg, 51%) as a yellow solid. ¹H-NMR (CDCl₃, Varian, 400 MHz): δ 1.46 (9H, s), 3.45 (4H, brs), 3.65 (2H brs), 3.82 (2H, brs), 5.27 (2H, brs), 5.98 (1H, d, J=5.6 Hz), 7.15 (1H, t, J=8.4 Hz), 7.23-7.26 (2H, m), 7.36 (1H, d, J=9.2 Hz), 7.58-7.62 (2H, m), 7.92-7.95 (2H, m), 8.24 (1H, d, J=4.0 Hz), 7.41 (1H, d, J=4.4 Hz), 11.82 (1H, brs).

### Step B: N-(4-(2-Amino-3-(3-oxo-3-(piperazin-1-yl)prop-1-ynyl)pyridin-4-yloxy)-3-fluorophenyl)-2-(4-fluorophenyl)-3-oxo-2,3-dihydropyridazine-4-carboxamide

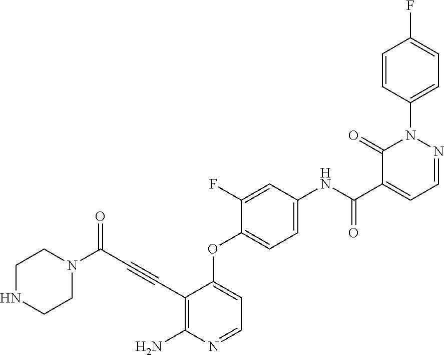 [SMILES: Nc1nccc(Oc2ccc(NC(=O)c3ccnn(-c4ccc(F)cc4)c3=O)cc2F)c1C#CC(=O)N1CCNCC1]

To a solution of tert-butyl 4-(3-(2-amino-4-(2-fluoro-4-(2-(4-fluorophenyl)-3-oxo-2,3-dihydropyridazine-4-carboxamido)phenoxy)pyridine-3-yl)piperazine-1-carboxylate (476 mg, 0.71 mmol) in DCM (4 mL) was added TFA (1.09 mL, 14.2 mmol) at room temperature. The reaction mixture was stirred for 3 h at room temperature. The reaction mixture was concentrated in vacuo, and diluted with DCM, and then neutralized with TEA. The mixture was stirred for 10 min at room temperature and concentrated in vacuo. The residue was triturated with DCM to afford the N-(4-(2-amino-3-(3-oxo-3-(piperazin-1-yl)prop-1-ynyl)pyridin-4-yloxy)-3-fluorophenyl)-2-(4-fluorophenyl)-3-oxo-2,3-dihydropyridazine-4-carboxamide (306 mg, 75%) as a yellow solid. ¹H-NMR (DMSO-d₆, Varian, 400 MHz): δ 3.09 (2H, brs), 3.18 (2H brs), 3.71 (2H, brs), 3.95 (2H, brs), 5.91 (1H, d, J=5.6 Hz), 6.67 (2H, brs), 7.38-7.44 (3H, m), 7.56 (1H, d, J=8.4 Hz), 7.66-7.70 (2H, m), 8.00 (1H, d, J=10.4 Hz), 8.25 (1H, d, J=4.0 Hz), 8.38 (1H, d, J=4.4 Hz), 8.69 (1H, brs), 11.67 (1H, brs). *NH peak was not observed.

### Example 14

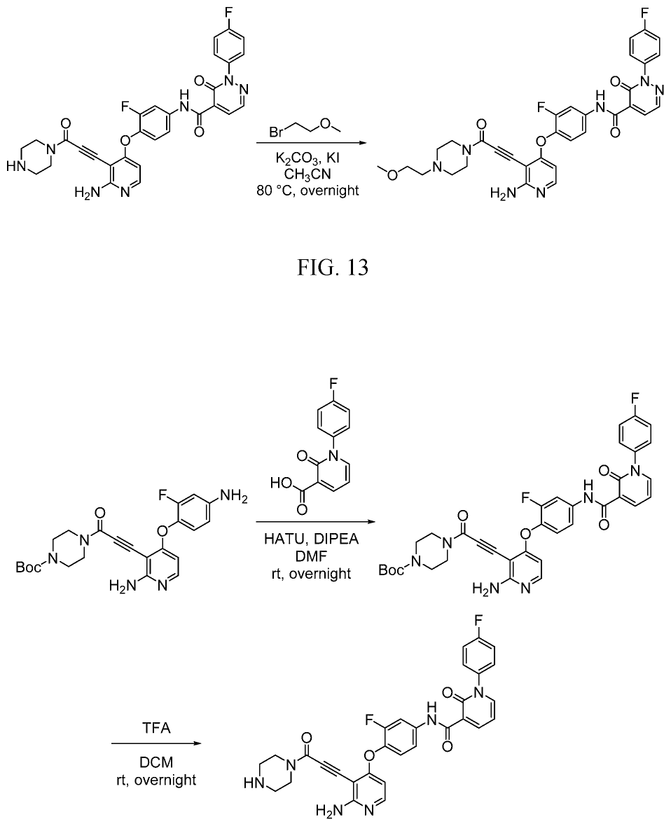

This example describes the synthesis of N-(4-(2-amino-3-(3-(4-(2-methoxyethyl)piperazin-1-yl)-3-oxoprop-1-ynyl)pyridin-4-yloxy)-3-fluorophenyl)-2-(4-fluorophenyl)-3-oxo-2,3-dihydropyridazine-4-carboxamide in an aspect of the invention. See FIG. 13.

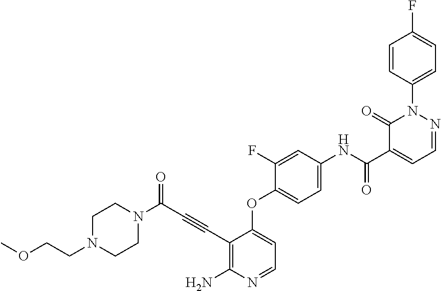 [SMILES: COCCN1CCN(C(=O)C#Cc2c(Oc3ccc(NC(=O)c4ccnn(-c5ccc(F)cc5)c4=O)cc3F)ccnc2N)CC1]

A mixture of N-(4-(2-amino-3-(3-oxo-3-(piperazin-1-yl)prop-1-ynyl)pyridin-4-yloxy)-3-fluorophenyl)-2-(4-fluorophenyl)-3-oxo-2,3-dihydropyridazine-4-carboxamide (Example 13, 40.0 mg, 0.07 mmol), 1-bromo-2-methoxyethane (8.75 mg, 0.06 mmol), potassium iodide (11.6 mg, 0.07 mmol) and K₂CO₃ (9.67 mg, 0.07 mmol) in CH₃CN (2 mL) was heated overnight at 80° C. in a sealed vessel. After being cooled to room temperature, EtOAc and water were poured into the reaction mixture and the separated aqueous layer was extracted with EtOAc. The combined organic layer was dried over Na₂SO₄, filtered, and concentrated in vacuo. The residue was purified by column chromatography (EtOAc) to afford the N-(4-(2-amino-3-(3-(4-(2-methoxyethyl)piperazin-1-yl)-3-oxoprop-1-ynyl)pyridin-4-yloxy)-3-fluorophenyl)-2-(4-fluorophenyl)-3-oxo-2,3-dihydropyridazine-4-carboxamide (43.0 mg, 98%) as a pale yellow solid. ¹H-NMR (CDCl₃, Varian, 400 MHz): δ 2.59 (4H, m), 2.59 (2H, t, J=5.2 Hz), 3.35 (3H, s), 3.50 (2H, t, J=4.8 Hz), 3.72 (2H, m), 3.88 (2H, m), 5.26 (2H, s), 5.98 (1H, d, J=6.0 Hz), 7.17 (1H, t, J=8.4 Hz), 7.23-7.26 (2H, m), 7.35 (1H, d, J=8.8 Hz), 7.60 (2H, q, J=4.4 Hz), 7.91-7.94 (2H, m), 8.24 (1H, d, J=4.0 Hz), 8.41 (1H, d, J=4.4 Hz), 11.82 (1H, brs).

### Example 15

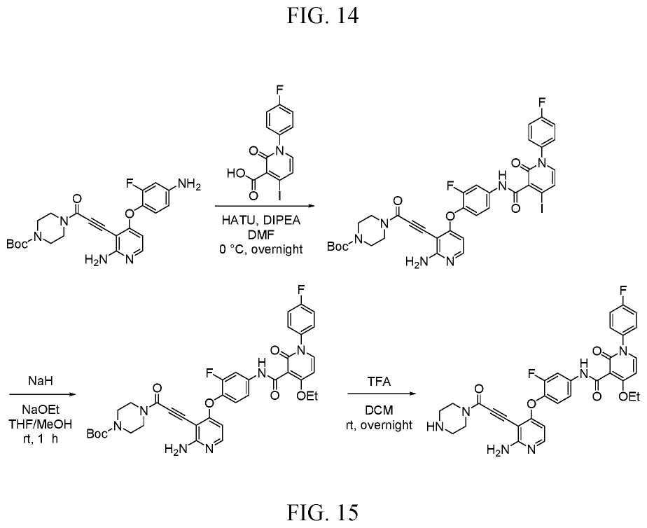

This example describes the synthesis of N-(4-(2-amino-3-(3-oxo-3-(piperazin-1-yl)prop-1-ynyl)pyridin-4-yloxy)-3-fluorophenyl)-1-(4-fluorophenyl)-2-oxo-1,2-dihydropyridine-3-carboxamide in an aspect of the invention. See FIG. 14.

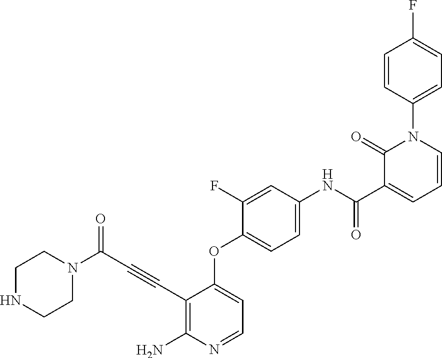 [SMILES: Nc1nccc(Oc2ccc(NC(=O)c3cccn(-c4ccc(F)cc4)c3=O)cc2F)c1C#CC(=O)N1CCNCC1]

### Step A: tert-Butyl 4-(3-(2-amino-4-(2-fluoro-4-(1-(4-fluorophenyl)-2-oxo-1,2-dihydropyridine-3-carboxamido)phenoxy)pyridin-3-yl)propioloyl)piperazine-1-carboxylate

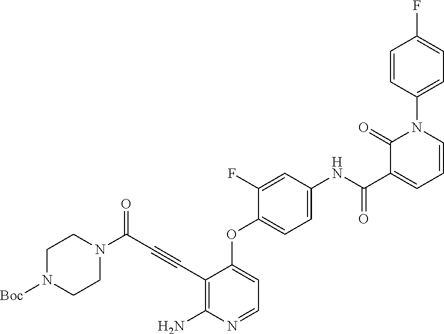 [SMILES: CC(C)(C)OC(=O)N1CCN(C(=O)C#Cc2c(Oc3ccc(NC(=O)c4cccn(-c5ccc(F)cc5)c4=O)cc3F)ccnc2N)CC1]

A mixture of tert-butyl 4-(3-(2-amino-4-(4-amino-2-fluorophenoxy)pyridin-3-yl)propioloyl)piperazine-1-carboxylate (intermediate 20, 100 mg, 0.22 mmol), 1-(4-fluorophenyl)-2-oxo-1,2-dihydropyridine-3-carboxylic acid (intermediate 16, 77.0 mg, 0.33 mmol), HATU (150 mg, 0.33 mmol), and DIPEA (153 μL, 0.88 mmol) in DMF (2.2 mL) was stirred for 2 h at room temperature. The mixture was partitioned between water and EtOAc. The organic layer was washed with water and brine, dried over Na₂SO₄, filtered, and concentrated in vacuo. The residue was purified by column chromatography (EtOAc/MeOH=97/3 to 95/5) to afford the tert-Butyl 4-(3-(2-amino-4-(2-fluoro-4-(1-(4-fluorophenyl)-2-oxo-1,2-dihydropyridine-3-carboxamido)phenoxy)pyridin-3-yl)propioloyl)piperazine-1-carboxylate (156 mg, 70%) as a brown solid. 1H-NMR (CDCl₃, Varian, 400 MHz): δ 1.46 (9H, s), 3.45 (4H, brs), 3.65 (2H brs), 3.82 (2H, brs), 5.29 (2H, brs), 5.98 (1H, d, J=6.0 Hz), 6.64 (1H, t, J=6.8 Hz), 7.11 (1H, t, J=8.8 Hz), 7.26-7.30 (3H, m), 7.33 (1H, d, J=8.8 Hz), 7.40-7.42 (2H, m), 7.64 (1H, d, J=6.4 Hz), 7.90-7.96 (2H, m), 12.01 (1H, brs).

### Step B: N-(4-(2-Amino-3-(3-oxo-3-(piperazin-1-yl)prop-1-ynyl)pyridin-4-yloxy)-3-fluorophenyl)-1-(4-fluorophenyl)-2-oxo-1,2-dihydropyridine-3-carboxamide

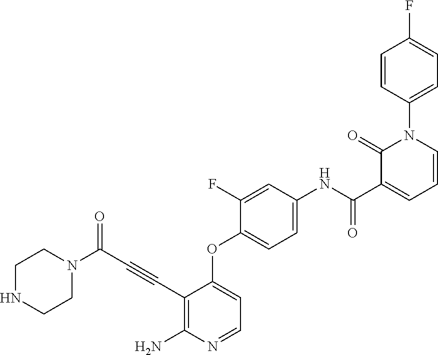 [SMILES: Nc1nccc(Oc2ccc(NC(=O)c3cccn(-c4ccc(F)cc4)c3=O)cc2F)c1C#CC(=O)N1CCNCC1]

To a solution of tert-Butyl 4-(3-(2-amino-4-(2-fluoro-4-(1-(4-fluorophenyl)-2-oxo-1,2-dihydropyridine-3-carboxamido)phenoxy)pyridin-3-yl)propioloyl)piperazine-1-carboxylate (103 mg, 0.15 mmol) in DMF (2 mL) was added TFA (476 μL, 6.17 mmol) at room temperature. The reaction mixture was stirred overnight at room temperature. The reaction mixture was concentrated in vacuo, the residue was diluted with DCM, and then neutralized with TEA. The mixture was stirred at room temperature for 10 minutes and concentrated in vacuo. The residue was purified by column chromatography on NH—SiO₂ (EtOAc/MeOH=95/5) to afford the N-(4-(2-Amino-3-(3-oxo-3-(piperazin-1-yl)prop-1-ynyl)pyridin-4-yloxy)-3-fluorophenyl)-1-(4-fluorophenyl)-2-oxo-1,2-dihydropyridine-3-carboxamide (54.6 mg, 62%) as a yellow solid. 1H-NMR (CDCl₃, Varian, 400 MHz): δ 2.86 (4H, brs), 3.65 (2H, t, J=5.0 Hz), 3.82 (2H, t, J=4.6 Hz), 5.28 (2H, brs), 5.99 (1H, d, J=5.6 Hz), 6.63 (1H, t, J=7.0 Hz), 7.10 (1H, t, J=8.4 Hz), 7.26-7.33 (3H, m), 7.39-7.43 (2H, m), 7.64 (1H, dd, J=6.4, 2.0 Hz), 7.90-7.95 (2H, m), 8.75 (1H, dd, J=7.2, 1.6 Hz), 12.00 (1H, brs). *NH peak was not observed.

### Example 16

This example describes the synthesis of N-(4-(2-amino-3-(3-oxo-3-(piperazin-1-yl)prop-1-ynyl)pyridin-4-yloxy)3-fluorophenyl)-4-ethoxy-1-(4-fluorophenyl)-2-oxo-1,2-dihydropyridine-3-carboxamide in an aspect of the invention. See FIG. 15.

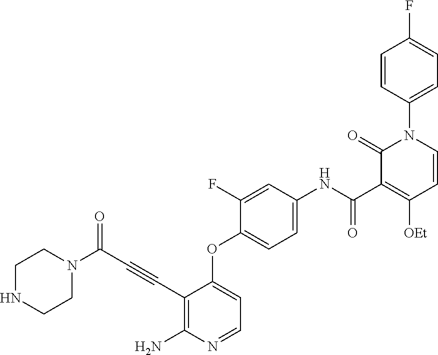 [SMILES: CCOc1ccn(-c2ccc(F)cc2)c(=O)c1C(=O)Nc1ccc(Oc2ccnc(N)c2C#CC(=O)N2CCNCC2)c(F)c1]

### Step A: tert-Butyl 4-(3-(2-amino-4-(2-fluoro-4-(1-(4-fluorophenyl)-4-iodo-2-oxo-1,2-dihydropyridine-3-carboxamido)phenoxy)pyridin-3-yl)piperazine-1-carboxylate

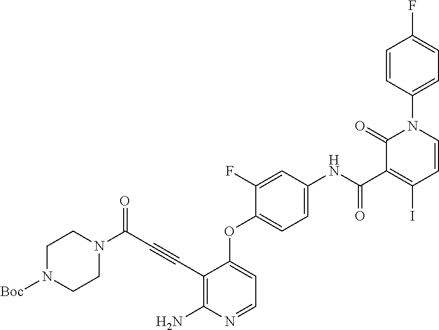 [SMILES: CC(C)(C)OC(=O)N1CCN(C(=O)C#Cc2c(Oc3ccc(NC(=O)c4c(I)ccn(-c5ccc(F)cc5)c4=O)cc3F)ccnc2N)CC1]

A mixture of tert-butyl 4-(3-(2-amino-4-(4-amino-2-fluorophenoxy)pyridin-3-yl)propioloyl)piperazine-1-carboxylate (intermediate 20, 110 mg, 0.24 mmol), 1-(4-(fluorophenyl)-4-iodo-2-oxo-1,2-dihydropyridine-3-carboxylic acid (intermediate 17, 130 mg, 0.36 mmol), HATU (138 mg, 0.36 mmol), and DIPEA (0.17 mL, 0.97 mmol) in DMF (2 mL) was stirred for 1 h at 0° C. EtOAc and water were poured into the reaction mixture and the separated aqueous layer was extracted with EtOAc. The combined organic layer was dried over Na₂SO₄, filtered, and concentrated in vacuo. The residue was purified by column chromatography (EtOAc/MeOH=97/3 to 95/5) to afford the tert-butyl 4-(3-(2-amino-4-(2-fluoro-4-(1-(4-fluorophenyl)-4-iodo-2-oxo-1,2-dihydropyridine-3-carboxamido)phenoxy)pyridin-3-yl)piperazine-1-carboxylate (72 mg, 37%) as a yellow solid. ¹H-NMR (CDCl₃, Varian, 400 MHz): δ 1.45 (9H, s), 3.45 (4H, m), 3.64 (2H, brs), 3.82 (2H, brs), 5.31 (2H, brs), 5.92 (1H, d, J=6.0 Hz), 6.51 (1H, t, J=7.2 Hz), 7.07-7.31 (3H, m), 7.39 (1H, brs), 7.68 (1H, d, J=7.6 Hz), 7.92-7.91 (2H, m), 8.14 (1H, d, J=8.4 Hz), 8.50 (1H, d, J=4.4 Hz), 11.56 (1H, s).

### Step B: tert-Butyl (4-(3-(2-amino-4-(4-(4-ethoxy-1-(4-fluorophenyl)-2-oxo-1,2-dihydropyridine-3-carboxamido)-2-flouorophenoxy)pyridine-3-yl)propioloyl)piperazine-1-carboxylate

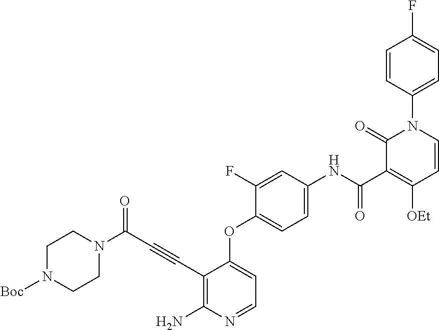 [SMILES: CCOc1ccn(-c2ccc(F)cc2)c(=O)c1C(=O)Nc1ccc(Oc2ccnc(N)c2C#CC(=O)N2CCN(C(=O)OC(C)(C)C)CC2)c(F)c1]

Sodium hydride (5.11 mg, 0.12 mmol, 55%) was added slowly in THF/EtOH (v/v=1:1, 2 mL) under N₂ atmosphere and the mixture was stirred for 5 min at room temperature. The reaction mixture was added to a solution of tert-butyl 4-(3-(2-amino-4-(2-fluoro-4-(1-(4-fluorophenyl)-4-iodo-2-oxo-1,2-dihydropyridine-3-carboxamido)phenoxy)pyridin-3-yl)piperazine-1-carboxylate (72 mg, 0.09 mmol) in THF/EtOH (v/v=1/1, 2 mL) and stirred for 1 h at room temperature. The reaction mixture was concentrated in vacuo. The residue was partitioned with EtOAc and water. The aqueous layer was extracted with EtOAc. The combined organic layer was washed with brine, dried over Na₂SO₄, filtered, and concentrated in vacuo. The residue was purified by column chromatography on SiO₂ (EtOAc/MeOH=95/5) to afford the tert-butyl (4-(3-(2-amino-4-(4-(4-ethoxy-1-(4-fluorophenyl)-2-oxo-1,2-dihydropyridine-3-carboxamido)-2-flouorophenoxy)pyridine-3-yl)propioloyl)piperazine-1-carboxylate (16 mg, 25%) as a yellow solid. ¹H-NMR (CDCl₃, Varian, 400 MHz): δ 1.45 (9H, s), 1.59 (3H, d, J=6.8 Hz), 3.45 (4H, s), 3.65 (2H, s), 3.82 (2H, s), 4.34-4.39 (2H, m), 5.23 (2H, s), 5.96 (1H, d, J=5.6 Hz), 6.37 (1H, d, J=8.0 Hz), 7.06 (1H, t, J=8.4 Hz), 7.22-7.29 (3H, m), 7.34-7.38 (2H, m), 7.52 (1H, d, J=7.6 Hz), 7.89-7.94 (2H, m), 11.65 (1H, s).

### Step C: N-(4-(2-Amino-3-(3-oxo-3-(piperazin-1-yl)prop-1-ynyl)pyridin-4-yloxy)₃-fluorophenyl)-4-ethoxy-1-(4-fluorophenyl)-2-oxo-1,2-dihydropyridine-3-carboxamide

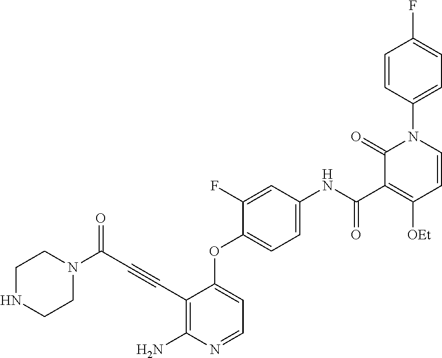 [SMILES: CCOc1ccn(-c2ccc(F)cc2)c(=O)c1C(=O)Nc1ccc(Oc2ccnc(N)c2C#CC(=O)N2CCNCC2)c(F)c1]

To a solution of tert-butyl (4-(3-(2-amino-4-(4-(4-ethoxy-1-(4-fluorophenyl)-2-oxo-1,2-dihydropyridine-3-carboxamido)-2-flouorophenoxy)pyridine-3-yl)propioloyl)piperazine-1-carboxylate (16 mg, 0.02 mmol) in DCM (2 mL) was added TFA (0.07 mL, 0.10 mmol) and stirred for 2 h. at room temperature The reaction mixture was concentrated in vacuo. The residue was diluted with DCM, and then TEA was added to the mixture until pH 7.0. The mixture was stirred at room temperature for 10 min and concentrated in vacuo. The residue was purified by column chromatography on NH—SiO₂ (EtOAc/MeOH=95/5) to afford the N-(4-(2-amino-3-(3-oxo-3-(piperazin-1-yl)prop-1-ynyl)pyridin-4-yloxy)₃-fluorophenyl)-4-ethoxy-1-(4-fluorophenyl)-2-oxo-1,2-dihydropyridine-3-carboxamide (11 mg, 81%) as a yellow solid. ¹H-NMR (CDCl₃, Varian, 400 MHz): δ 1.58 (3H, d, J=6.8 Hz), 2.86 (4H, s), 3.65 (2H, s), 3.82 (2H, s), 4.33-4.39 (2H, m), 5.24 (2H, s), 5.37 (1H, d, J=6.0 Hz), 6.37 (1H, d, J=8.0 Hz), 7.05 (1H, t, J=8.8 Hz), 7.24-7.35 (4H, m), 7.37 (1H, d, J=4.8 Hz), 7.52 (1H, d, J=7.6 Hz), 7.91 (2H, d, J=10.4 Hz), 11.64 (1H, s). * NH peak was not observed.

### Example 17

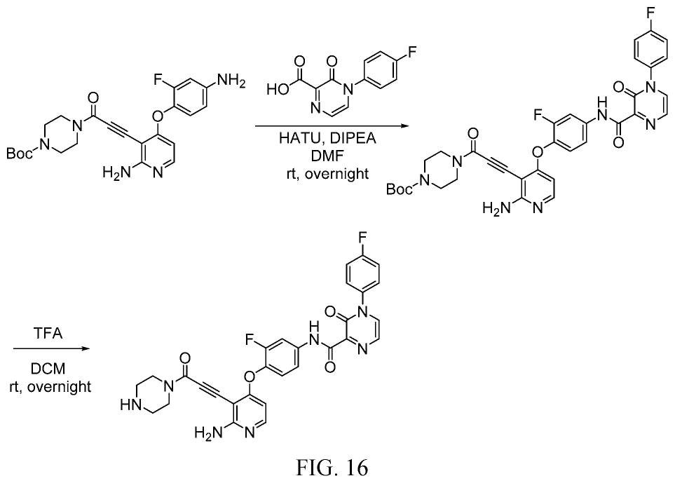

This example describes the synthesis of N-(4-(2-amino-3-(3-oxo-3-(piperazin-1-yl)prop-1-ynyl)pyridin-4-yloxy)-3-fluorophenyl)-4-(4-fluorophenyl)-3-oxo-3,4-dihydropyrazine-2-carboxamide in an aspect of the invention. See FIG. 16.

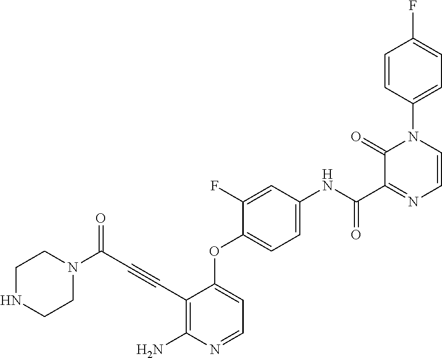 [SMILES: Nc1nccc(Oc2ccc(NC(=O)c3nccn(-c4ccc(F)cc4)c3=O)cc2F)c1C#CC(=O)N1CCNCC1]

### Step A: tert-Butyl 4-(3-(2-amino-4-(2-fluoro-4-(4-(4-fluorophenyl)-3-oxo-3,4-dihydropyrazine-2-carboxamido)phenoxy)pyridin-3-yl)propioloyl)piperazine-1-carboxylate

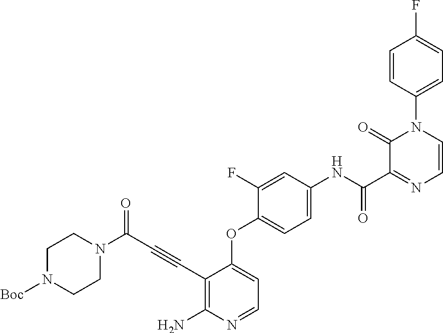 [SMILES: CC(C)(C)OC(=O)N1CCN(C(=O)C#Cc2c(Oc3ccc(NC(=O)c4nccn(-c5ccc(F)cc5)c4=O)cc3F)ccnc2N)CC1]

A mixture of tert-butyl 4-(3-(2-amino-4-(4-amino-2-fluorophenoxy)pyridin-3-yl)propioloyl)piperazine-1-carboxylate (intermediate 20, 100 mg, 0.22 mmol), 4-(4-fluorophenyl)-3-oxo-3,4-dihydropyrazine-2-carboxylic acid (intermediate 18, 77 mg, 0.33 mmol), HATU (125 mg, 0.33 mmol), and DIPEA (0.15 mL, 0.88 mmol) in DMF (2 mL) was stirred overnight at room temperature. EtOAc and water were poured into the reaction mixture and the separated aqueous layer was extracted with EtOAc. The combined organic layer was dried over Na₂SO₄, filtered, and concentrated in vacuo. The residue was purified by column chromatography (EtOAc/MeOH=5/1) to afford the tert-butyl 4-(3-(2-amino-4-(2-fluoro-4-(4-(4-fluorophenyl)-3-oxo-3,4-dihydropyrazine-2-carboxamido)phenoxy)pyridin-3-yl)propioloyl)piperazine-1-carboxylate (140 mg, 95%) as a pale yellow solid. ¹H-NMR (DMSO-d₆, Varian, 400 MHz): δ 2.81 (9H, s), 3.45 (4H, m), 3.65 (2H, m), 3.82 (2H, m), 5.26 (2H, brs), 6.00 (1H, d, J=6.0 Hz), 7.16 (1H, t, J=9.6 Hz), 7.26-7.34 (2H, m), 7.39 (1H, d, J=6.8 Hz), 7.44-7.46 (2H, m), 7.52 (1H, d, J=3.2 Hz), 7.93 (2H, t, J=6.0 Hz), 8.02 (1H, s), 11.81 (1H, s).

### Step B: N-(4-(2-Amino-3-(3-oxo-3-(piperazin-1-yl)prop-1-ynyl)pyridin-4-yloxy)-3-fluorophenyl)-4-(4-fluorophenyl)-3-oxo-3,4-dihydropyrazine-2-carboxamide

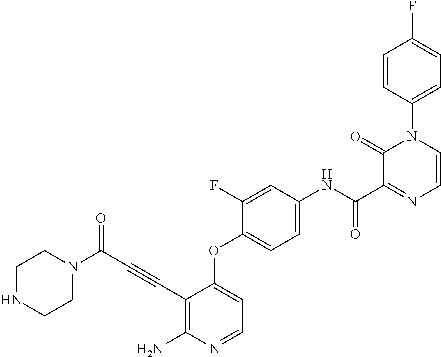 [SMILES: Nc1nccc(Oc2ccc(NC(=O)c3nccn(-c4ccc(F)cc4)c3=O)cc2F)c1C#CC(=O)N1CCNCC1]

TFA (0.16 mL, 2.10 mmol) was added to a solution of tert-butyl 4-(3-(2-amino-4-(2-fluoro-4-(4-(4-fluorophenyl)-3-oxo-3,4-dihydropyrazine-2-carboxamido)phenoxy)pyridin-3-yl)propioloyl)piperazine-1-carboxylate (140 mg, 0.21 mmol) in DCM (4 mL). The mixture was stirred overnight at room temperature. The excess TFA was removed by evaporation, and DCM was poured into the residue. The mixture was neutralized with saturated NaHCO₃(aq.) at 0° C. The separated aqueous layer was extracted with DCM, and the combined organic layer was dried over Na₂SO₄, filtered, and concentrated in vacuo. The residue was purified by column chromatography (EtOAc/MeOH=5/1) to afford the N-(4-(2-amino-3-(3-oxo-3-(piperazin-1-yl)prop-1-ynyl)pyridin-4-yloxy)-3-fluorophenyl)-4-(4-fluorophenyl)-3-oxo-3,4-dihydropyrazine-2-carboxamide (58.7 mg, 49%) as a pale yellow solid. ¹H-NMR (DMSO-d₆, Varian, 400 MHz): δ 2.83 (4H, m), 3.66 (2H, m), 3.81 (2H, m), 5.24 (2H, brs), 6.00 (1H, d, J=5.6 Hz), 7.14 (2H, t, J=8.8 Hz), 7.32 (2H, t, J=8.0 Hz), 7.37 (1H, d, J=8.4 Hz), 7.44-7.46 (2H, m), 7.51 (1H, d, J=4.4 Hz), 7.91-7.97 (2H, m), 10.80 (1H, s). * NH peak was not observed.

### Example 18

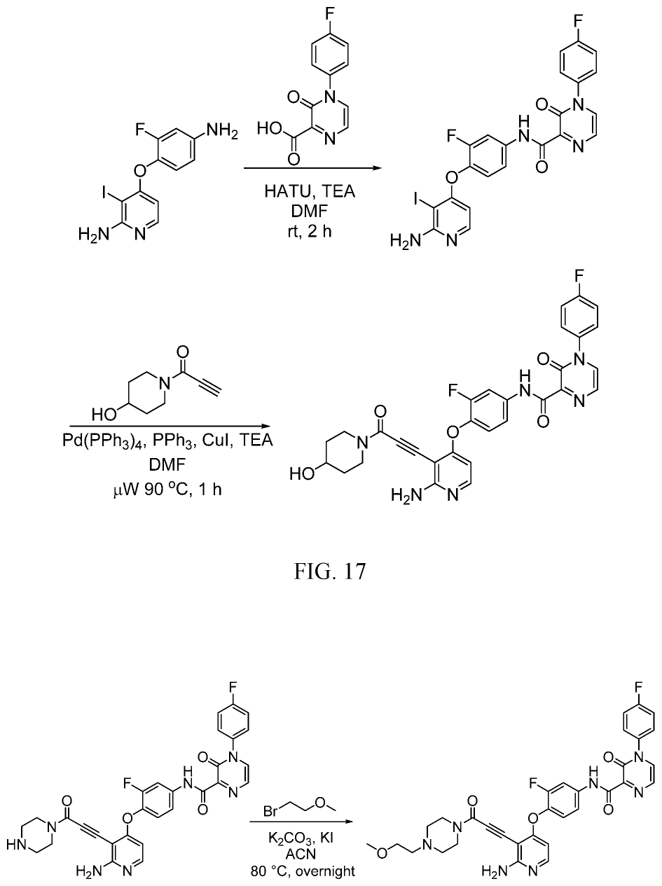

This example describes the synthesis of N-(4-(2-amino-3-(3-(4-hydroxypiperidin-1-yl)-3-oxoprop-1-ynyl)pyridin-4-yloxy)-3-fluorophenyl)-4-(4-fluorophenyl)-3-oxo-3,4-dihydropyrazine-2-carboxamide in an aspect of the invention. See FIG. 17.

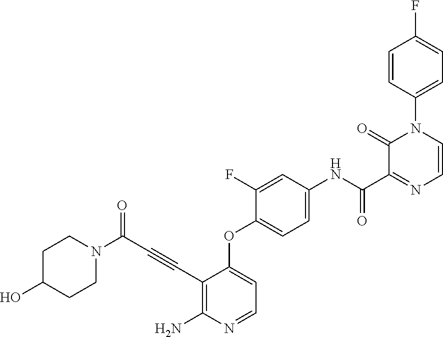 [SMILES: Nc1nccc(Oc2ccc(NC(=O)c3nccn(-c4ccc(F)cc4)c3=O)cc2F)c1C#CC(=O)N1CCC(O)CC1]

### Step A: N-(4-(2-Amino-3-iodopyridin-4-yloxy)-3-fluorophenyl)-4-(4-fluorophenyl)-3-oxo-3,4-dihydropyrazine-2-carboxamide

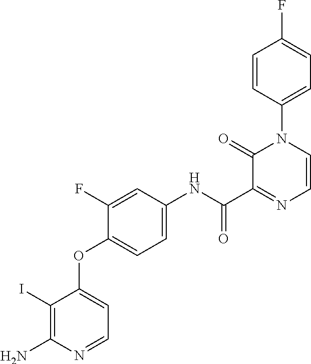 [SMILES: Nc1nccc(Oc2ccc(NC(=O)c3nccn(-c4ccc(F)cc4)c3=O)cc2F)c1I]

A mixture of 4-(4-amino-2-fluorophenoxy)-3-iodopyridin-2-amine (step D of intermediate 19, 500 mg, 1.45 mmol), 4-(4-fluorophenyl)-3-oxo-3,4-dihydropyrazine-2-carboxylic acid (intermediate 18, 509 mg, 2.17 mmol), HATU (606 mg, 1.59 mmol), and TEA (505 μL, 3.62 mmol) in DMF (7 mL) was stirred for 2 h at room temperature. The reaction mixture was partitioned between water and EtOAc. The aqueous layer was extracted with EtOAc. The combined organic layer was washed with water and brine, dried over Na₂SO₄, filtered, and concentrated in vacuo. The residue was triturated with Et₂O to afford the N-(4-(2-amino-3-iodopyridin-4-yloxy)-3-fluorophenyl)-4-(4-fluorophenyl)-3-oxo-3,4-dihydropyrazine-2-carboxamide (826 mg, quant.) as a yellow solid. ¹H-NMR (CDCl₃, Varian, 400 MHz): δ 5.08 (2H, brs), 5.90 (1H, d, J=5.2 Hz), 7.15 (1H, t, J=8.8 Hz), 7.31 (2H, t, J=8.4 Hz), 7.38 (1H, d, J=8.8 Hz), 7.43-7.47 (2H, m), 7.50 (1H, d, J=4.0 Hz), 7.79 (1H, d, J=5.6 Hz), 7.93 (1H, s), 7.94 (1H, dd, J=14.4, 2.0 Hz), 11.79 (1H, brs).

### Step B: N-(4-(2-Amino-3-(3-(4-hydroxypiperidin-1-yl)-3-oxoprop-1-ynyl)pyridin-4-yloxy)-3-fluorophenyl)-4-(4-fluorophenyl)-3-oxo-3,4-dihydropyrazine-2-carboxamide

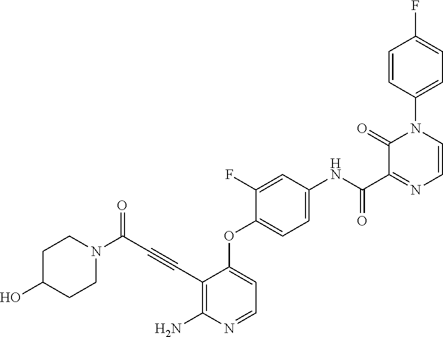 [SMILES: Nc1nccc(Oc2ccc(NC(=O)c3nccn(-c4ccc(F)cc4)c3=O)cc2F)c1C#CC(=O)N1CCC(O)CC1]

To a solution of N-(4-(2-amino-3-iodopyridin-4-yloxy)-3-fluorophenyl)-4-(4-fluorophenyl)-3-oxo-3,4-dihydropyrazine-2-carboxamide (200 mg, 0.36 mmol), 1-(4-hydroxypiperidin-1-yl)prop-2-yn-1-one (intermediate 7, 164 mg, 1.07 mmol), and TEA (174 L, 1.25 mmol) in DMF (1.8 mL) were added CuI (14.0 mg, 0.071 mmol), PPh₃ (28.0 mg, 0.107 mmol) and Pd(PPh₃)₄ (41.0 mg, 0.036 mmol) under N₂ at room temperature. The reaction mixture was subjected to microwave irradiation for 1 h at 90° C. The mixture was concentrated in vacuo, and the residue was purified by column chromatography on NH—SiO₂ (EtOAc/MeOH=95/5 to 93/7) to afford the N-(4-(2-amino-3-(3-(4-hydroxypiperidin-1-yl)-3-oxoprop-1-ynyl)pyridin-4-yloxy)-3-fluorophenyl)-4-(4-fluorophenyl)-3-oxo-3,4-dihydropyrazine-2-carboxamide (33.0 mg, 16%) as a yellow solid. ¹H-NMR (DMSO-d₆, Varian, 400 MHz): δ 1.23-1.39 (2H, m), 1.72 (2H, brs), 3.14-3.20 (1H, m), 3.45-3.48 (1H, m), 3.72 (1H, brs), 3.85-3.89 (1H, m), 4.03-4.06 (1H, m), 4.78 (1H, brs), 5.92 (1H, d, J=6.4 Hz), 6.55 (2H, brs), 7.36-7.47 (3H, m), 7.46 (1H, d, J=9.2 Hz), 7.62-7.64 (3H, m), 7.89 (1H, d, J=5.6 Hz), 7.74 (1H, d, J=12.4 Hz), 7.99 (1H, s), 11.30 (1H, brs).

### Example 19

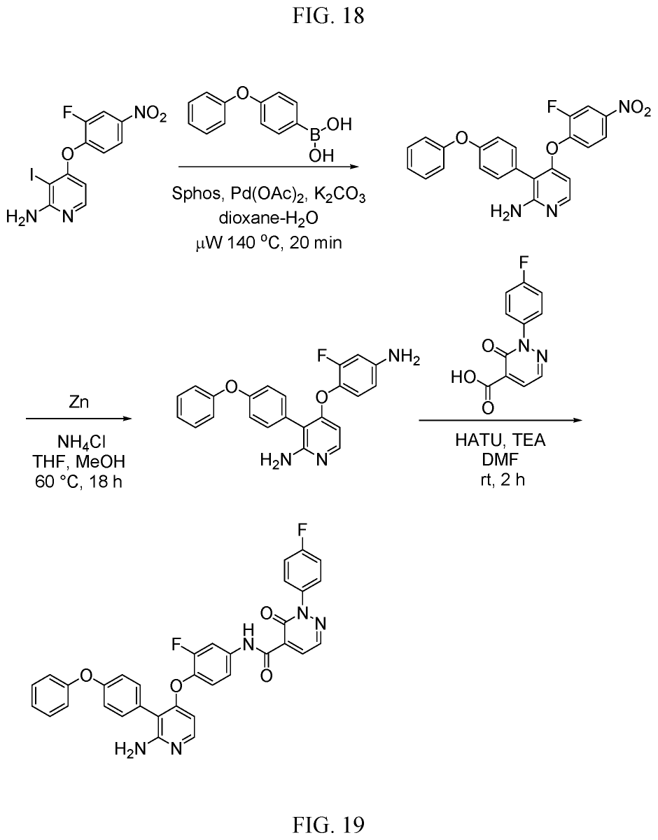

This example describes the synthesis of N-(4-(2-amino-3-(3-(4-(2-methoxyethyl)piperazin-1-yl)-3-oxoprop-1-ynyl)pyridin-4-yloxy)-3-fluorophenyl)-4-(4-fluorophenyl)-3-oxo-3,4-dihydropyrazine-2-carboxamide in an aspect of the invention. See FIG. 18.

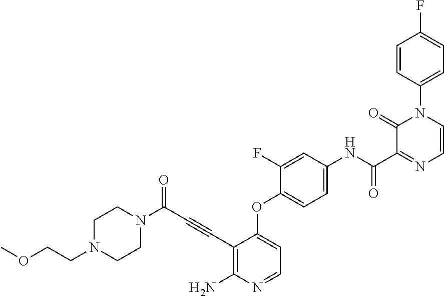 [SMILES: COCCN1CCN(C(=O)C#Cc2c(Oc3ccc(NC(=O)c4nccn(-c5ccc(F)cc5)c4=O)cc3F)ccnc2N)CC1]

A mixture of N-(4-(2-amino-3-(3-oxo-3-(piperazin-1-yl)prop-1-ynyl)pyridin-4-yloxy)-3-fluorophenyl)-4-(4-fluorophenyl)-3-oxo-3,4-dihydropyrazine-2-carboxamide (Example 17, 459 mg, 0.08 mmol), 1-bromo-2-methoxyethane (7.55 μL, 0.08 mmol), KI (13.0 mg, 0.08 mmol) and K₂CO₃ (11.0 mg, 0.08 mmol) in CH₃CN (2 mL) was stirred overnight at 80° C. in a sealed vial. The reaction mixture was partitioned with EtOAc and water, extracted with EtOAc. The combined organic layer was washed with brine, dried over Na₂SO₄, and concentrated in vacuo. The residue was purified by column chromatography on NH—SiO₂ (EtOAc/MeOH=95/5) to afford the N-(4-(2-amino-3-(3-(4-(2-methoxyethyl)piperazin-1-yl)-3-oxoprop-1-ynyl)pyridin-4-yloxy)-3-fluorophenyl)-4-(4-fluorophenyl)-3-oxo-3,4-dihydropyrazine-2-carboxamide (38.9 mg, 77%) as a yellow solid. ¹H-NMR (CDCl₃, Varian, 400 MHz): δ 2.51 (4H, brs), 2.59 (2H, t, J=5.2 Hz), 3.34 (3H, s), 3.50 (2H, t, J=5.2 Hz), 3.71 (2H, brs), 3.87 (2H, brs), 5.30 (2H, brs), 5.99 (1H, d, J=5.6 Hz), 7.13 (1H, t, J=8.8 Hz), 7.27-7.37 (3H, m), 7.44-7.48 (2H, m), 7.54 (1H, d, J=4.0 Hz), 7.91-7.97 (3H, m), 11.80 (1H, brs).

### Example 20

This example describes the synthesis of N-(4-(2-amino-3-(4-phenoxyphenyl)pyridin-4-yloxy)-3-fluorophenyl)-2-4-(fluorophenyl)-3-oxo-2,3-dihydropyridazine-4-carboxamide in an aspect of the invention. See FIG. 19.

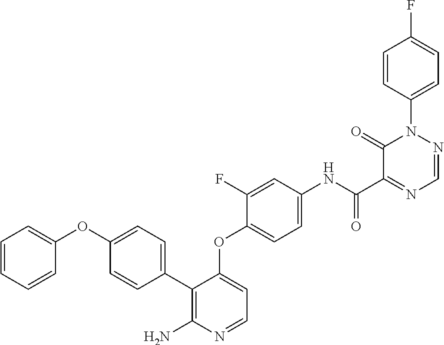 [SMILES: Nc1nccc(Oc2ccc(NC(=O)c3ncnn(-c4ccc(F)cc4)c3=O)cc2F)c1-c1ccc(Oc2ccccc2)cc1]

### Step A: 4-(2-Fluoro-4-nitrophenoxy)-3-(4-phenoxyphenyl)pyridine-2-amine

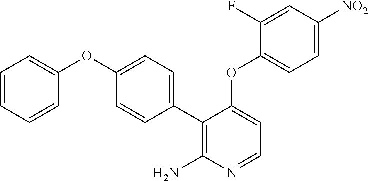 [SMILES: Nc1nccc(Oc2ccc([N+](=O)[O-])cc2F)c1-c1ccc(Oc2ccccc2)cc1]

To a solution of 4-(2-fluoro-4-nitrophenoxy)-3-iodopyridin-2-amine (step D of intermediate 19, 300 mg, 0.80 mmol) in dioxane/H₂O (v/v=1/1, 2.0 mL) were added 4-phenoxyphenylboronic acid (257 mg, 1.20 mmol), Sphos (32.8 mg, 0.08 mmol), K₂CO₃ (332 mg, 2.40 mmol) and Pd(OAc)₂ (9.00 mg, 0.04 mmol) under N₂ at room temperature. The reaction mixture was subjected to microwave irradiation for 20 min at 140° C. After being cooled at room temperature, Na₂SO₄ was added to the mixture which was subsequently filtered through a plug of silica gel and concentrated in vacuo. The residue was purified by medium pressure liquid chromatography (MPLC) (EtOAc/MeOH=9/1) to afford the 4-(2-fluoro-4-nitrophenoxy)-3-(4-phenoxyphenyl)pyridine-2-amine (290 mg, 87%) as a yellow solid. ¹H-NMR (CDCl₃, Varian, 400 MHz): δ 4.82 (2H, s), 6.27 (1H, d, J=6.0 Hz), 6.98-7.09 (4H, m), 7.11-7.16 (2H, m), 7.29-7.37 (4H, m), 7.88-8.05 (3H, m).

### Step B: 4-(4-Amino-2-fluorophenoxy)-3-(4-phenoxyphenyl)pyridine-2-amine

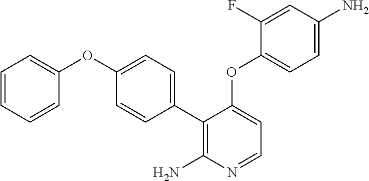 [SMILES: Nc1ccc(Oc2ccnc(N)c2-c2ccc(Oc3ccccc3)cc2)c(F)c1]

A mixture of 4-(2-fluoro-4-nitrophenoxy)-3-(4-phenoxyphenyl)pyridine-2-amine (290 mg, 0.69 mmol), zinc (450 mg, 6.95 mmol), and ammonium chloride (370 mg, 6.95 mmol) in THF/MeOH (v/v=1/1, 7.0 mL) was stirred for 1 h at 60° C. After being cooled at room temperature, the mixture was filtered and the filtrate was partitioned between EtOAc and saturated NaHCO₃(aq.). The aqueous layer was extracted with EtOAc. The combined organic layer was washed with brine, dried over Na₂SO₄, filtered, and concentrated in vacuo. The residue was purified by column chromatography on SiO₂ (EtOAc/MeOH=95/5) to afford the 4-(4-amino-2-fluorophenoxy)-3-(4-phenoxypheny)pyridin-2-amine (260 mg, 97%) as a yellow oil. ¹H-NMR (CDCl₃, Varian, 400 MHz): δ 6.97-7.04 (6H, m), 7.08-7.14 (4H, m), 7.31-7.37 (6H, m). * NH₂ peak was not observed.

### Step C: N-(4-(2-Amino-3-(4-phenoxyphenyl)pyridin-4-yloxy)-3-fluorophenyl)-2-4-(fluorophenyl)-3-oxo-2,3-dihydropyridazine-4-carboxamide

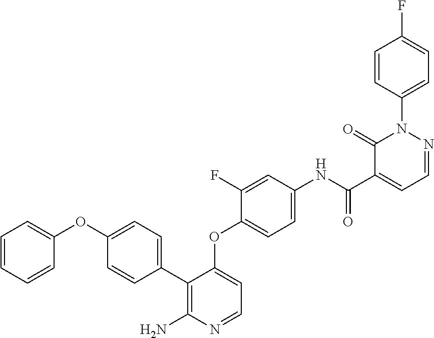 [SMILES: Nc1nccc(Oc2ccc(NC(=O)c3ccnn(-c4ccc(F)cc4)c3=O)cc2F)c1-c1ccc(Oc2ccccc2)cc1]

A mixture of 4-(4-amino-2-fluorophenoxy)-3-(4-phenoxypheny)pyridin-2-amine (260 mg, 0.67 mmol), 2-(4-fluorophenyl)-3-oxo-2,3-dihydropyridazine-4-carboxylic acid (intermediate 15, 238 mg, 1.01 mmol), HATU (283 mg, 0.74 mmol), and TEA (0.23 mL, 1.69 mmol) in DMF (5.0 mL) was stirred for 2 h at room temperature. Water was poured into the mixture, the precipitated product was collected by vacuum filtration, washed with water and ether. The solid was dried under vacuum to afford the N-(4-(2-amino-3-(4-phenoxyphenyl)pyridin-4-yloxy)-3-fluorophenyl)-2-4-(fluorophenyl)-3-oxo-2,3-dihydropyridazine-4-carboxamide (210 mg, 52%) as a yellow solid. ¹H-NMR (CDCl₃, Varian, 400 MHz): δ 4.52 (2H, s), 6.10 (1H, d, J=6.0), 7.03-7.11 (4H, m), 7.13-7.15 (1H, m), 7.20-7.22 (4H, m), 7.24-7.43 (4H, m), 7.56-7.61 (2H, m), 7.84 (1H, dd, J=12.4 Hz), 7.89 (1H, d, J=6.0 Hz), 8.21 (1H, d, J=4.4 Hz), 8.38 (1H, d, J=4.4 Hz), 11.73 (1H, s).

### Example 21

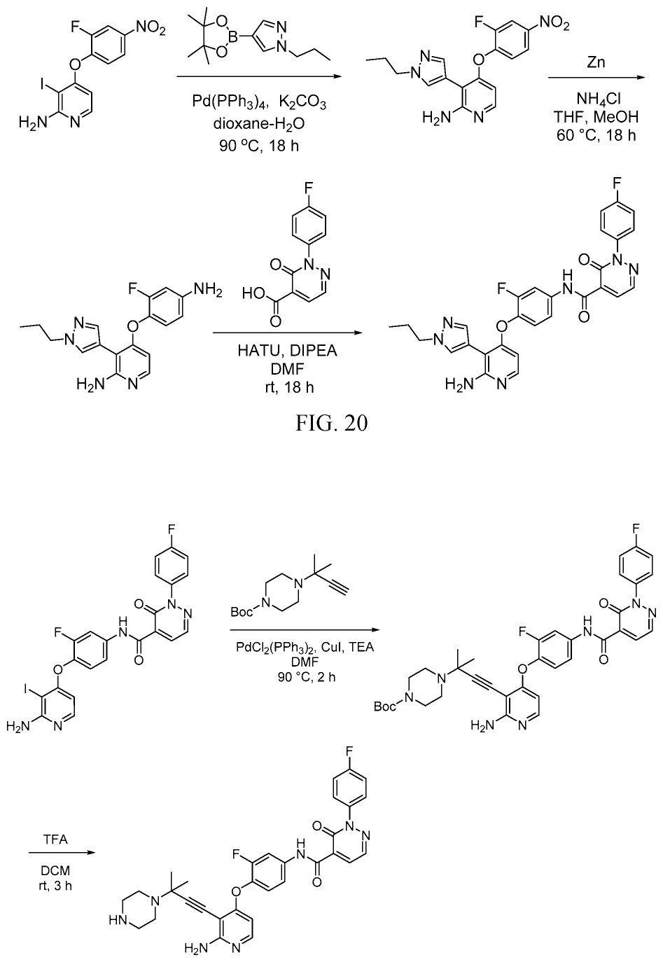

This example describes the synthesis of N-(4-(2-amino-3-(1-propyl-1H-pyrazol-4-yl)pyridin-4-yloxy)-3-fluorophenyl)-2-(4-fluorophenyl)-3-oxo-2,3-dihydropyridazine-4-carboxamide in an aspect of the invention. See FIG. 20.

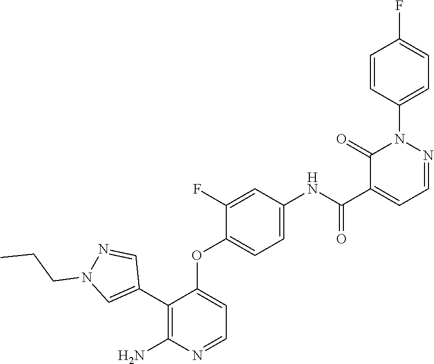 [SMILES: CCCn1cc(-c2c(Oc3ccc(NC(=O)c4ccnn(-c5ccc(F)cc5)c4=O)cc3F)ccnc2N)cn1]

### Step A: 4-(2-Fluoro-4-nitrophenoxy)-3-(1-propyl-1H-pyrazol-4-yl)pyridine-2-amine

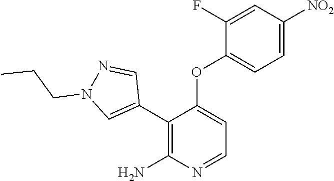 [SMILES: CCCn1cc(-c2c(Oc3ccc([N+](=O)[O-])cc3F)ccnc2N)cn1]

To a degassed solution of 4-(2-fluoro-4-nitrophenoxy)-3-iodopyridin-2-amine (200 mg, 0.533 mmol), 1-methyl-4-(4,4,5,5-tetramethyl-1,3,2-dixaborolan-yl)-1H-pyrazole (189 mg, 0.800 mmol) in 1,4-dioxane (4.0 mL) were added a degassed solution of K₂CO₃ (221 mg, 1.60 mmol) in H₂O (2.0 mL) and Pd(PPh₃)₄ (61 mg, 0.05 mmol). The reaction mixture was stirred for 18 h at 90° C. After being cooled at room temperature, to the mixture was added saturated NaHCO₃(aq.) and extracted with EtOAc. The combined organic layer was dried over Na₂SO₄, filtered, and concentrated in vacuo. The residue was purified by column chromatography on SiO₂ (DCM/MeOH=9/1) to afford the 4-(2-fluoro-4-nitrophenoxy)-3-(1-propyl-1H-pyrazol-4-yl)pyridine-2-amine (156 mg, 82%) as a yellow solid. ¹H-NMR (CDCl₃, Varian, 400 MHz): δ 0.86 (3H, t, J=7.6 Hz), 1.82-1.91 (2H, m), 4.08 (2H, t, J=7.2 Hz), 4.84 (2H, s), 6.30 (1H, d, J=5.2 Hz), 7.02 (1H, t, J=8.0 Hz), 7.58 (1H, s), 7.66 (1H, s), 7.96-8.04 (3H, m).

### Step B: 4-(4-Amino-2-fluorophenoxy)-3-(1-propyl-1H-pyrazol-4-yl)pyridin-2-amine

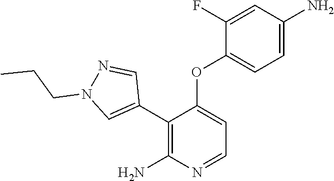 [SMILES: CCCn1cc(-c2c(Oc3ccc(N)cc3F)ccnc2N)cn1]

A mixture of 4-(2-fluoro-4-nitrophenoxy)-3-(1-propyl-1H-pyrazol-4-yl)pyridine-2-amine (156 mg, 0.43 mmol), zinc (285 mg, 4.37 mmol), and ammonium chloride (234 mg, 4.37 mmol) in THF/MeOH (v/v=1/1, 4.0 mL) was stirred for 18 h at 60° C. After being cooled at room temperature, the mixture was filtered and the filtrate was partitioned between EtOAc and saturated NaHCO₃(aq.). The aqueous layer was extracted with EtOAc. The combined organic layer was washed with brine, dried over Na₂SO₄, filtered, and concentrated in vacuo. The residue was purified by column chromatography on SiO₂ (EtOAc/MeOH=95/5) to afford the 4-(4-amino-2-fluorophenoxy)-3-(1-propyl-1H-pyrazol-4-yl)pyridin-2-amine (140 mg, 98%) as a yellow solid. ¹H-NMR (CD₃OD, Varian, 400 MHz): δ 0.93 (3H, t, J=7.6 Hz), 1.88-1.97 (2H, m), 4.17 (2H, t, J=7.2 Hz), 6.08 (1H, d, J=6.4 Hz), 6.49-6.55 (2H, m), 6.85-6.89 (1H, m), 7.54-7.57 (1H, m), 7.61-7.68 (1H, m), 7.70 (1H, s), 7.80 (1H, d, J=6.0 Hz), 7.87 (1H, s). * NH₂ peak was not observed.

### Step C: N-(4-(2-Amino-3-(1-propyl-1H-pyrazol-4-yl)pyridin-4-yloxy)-3-fluorophenyl)-2-(4-fluorophenyl)-3-oxo-2,3-dihydropyridazine-4-carboxamide

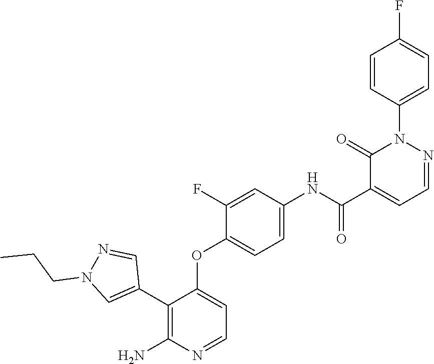 [SMILES: CCCn1cc(-c2c(Oc3ccc(NC(=O)c4ccnn(-c5ccc(F)cc5)c4=O)cc3F)ccnc2N)cn1]

A mixture of 4-(4-amino-2-fluorophenoxy)-3-(1-propyl-1H-pyrazol-4-yl)pyridin-2-amine (80 mg, 0.24 mmol), 2-(4-fluorophenyl)-3-oxo-2,3-dihydropyridazine-4-carboxylic acid (intermediate 15, 57 mg, 0.24 mmol), HATU (102 mg, 0.27 mmol) and DIPEA (0.1 mL, 0.611 mmol) in DMF (5.0 mL) was stirred for 18 h at room temperature. The reaction was quenched by water and extracted with EtOAc. The organic layer was dried over Na₂SO₄, filtered, and concentrated in vacuo. The residue was purified by column chromatography on SiO₂ (EtOAc/MeOH=9/1) to afford the N-(4-(2-amino-3-(1-propyl-1H-pyrazol-4-yl)pyridin-4-yloxy)-3-fluorophenyl)-2-(4-fluorophenyl)-3-oxo-2,3-dihydropyridazine-4-carboxamide (52 mg, 39%) as a white solid. ¹H-NMR (CDCl₃, Varian, 400 MHz): δ 0.94 (3H, t, J=7.2 Hz), 1.89-2.06 (2H, m), 4.14 (2H, t, J=7.6 Hz), 5.62 (2H, brs), 6.14 (1H, d, J=6.4 Hz), 7.07 (1H, t, J=8.4 Hz), 7.22-7.28 (2H, m), 7.31 (1H, d, J=8.8 Hz), 7.57-7.60 (2H, m), 7.69 (1H, s), 7.75 (1H, s), 7.82 (1H, d, J=6.0 Hz), 7.89 (1H, dd, J=12.2 Hz), 8.22 (1H, d, J=4.4 Hz), 7.39 (1H, d, J=4.0 Hz), 11.78 (1H, s).

### Example 22

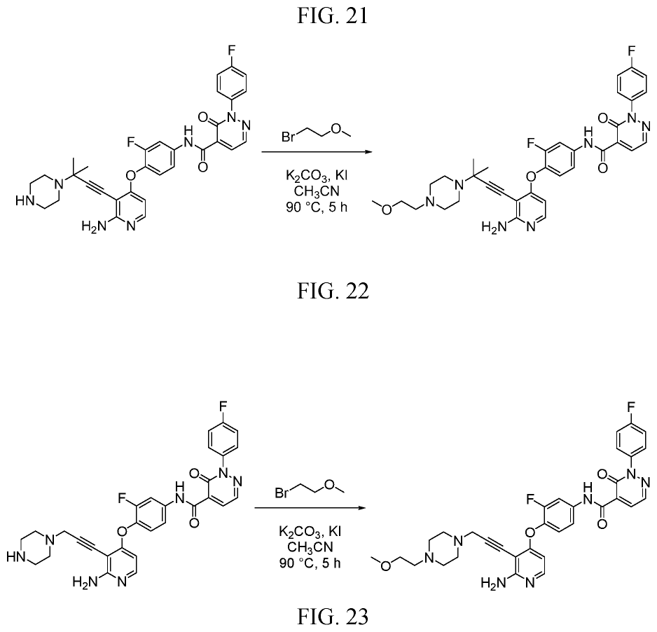

This example describes the synthesis of N-(4-(2-amino-3-(3-methyl-3-(piperazin-1-yl)but-1-ynyl)pyridin-4-yloxy)-3-fluorophenyl)-2-(4-fluorophenyl)-3-oxo-2,3-dihydropyridazine-4-carboxamide in an aspect of the invention. See FIG. 21.

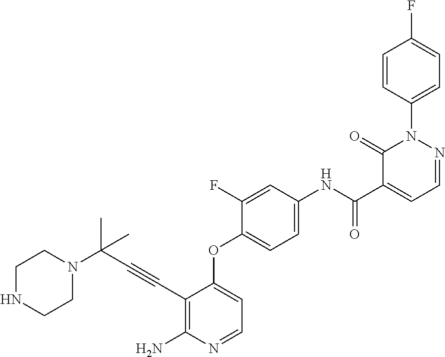 [SMILES: CC(C)(C#Cc1c(Oc2ccc(NC(=O)c3ccnn(-c4ccc(F)cc4)c3=O)cc2F)ccnc1N)N1CCNCC1]

### Step A: tert-Butyl 4-(4-(2-amino-4-(2-fluoro-4-(2-(4-fluorophenyl)-3-oxo-2,3-dihydropyridazine-4-carboxamido)phenoxy)pyridin-3-yl)-2-methylbut-3-yn-2-yl)piperazine-1-carboxylate

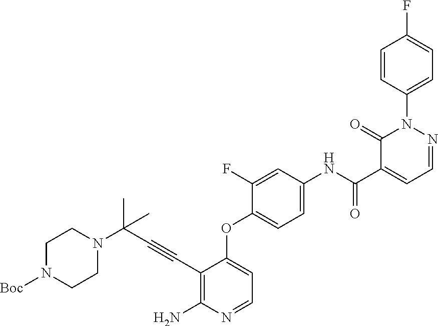 [SMILES: CC(C)(C)OC(=O)N1CCN(C(C)(C)C#Cc2c(Oc3ccc(NC(=O)c4ccnn(-c5ccc(F)cc5)c4=O)cc3F)ccnc2N)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 200 mg, 0.36 mmol), tert-butyl 4-(2-methylbut-3-yn-2-yl)piperazine-1-carboxylate (intermediate 12, 135 mg, 0.53 mmol), Pd(PPh₃)₄ (41.2 mg, 0.04 mmol), and copper(I) iodide (14.0 mg, 0.07 mmol) in DMF (3.0 mL) was purged with N₂. The reaction mixture was stirred for 2 h at 90° C. After cooled at room temperature, EtOAc and water were added the mixture, and the separated aqueous layer was extracted with EtOAc. The combined organic layer was dried over Na₂SO₄, filtered, and concentrated in vacuo. The residue was purified by MPLC (EtOAc to EtOAc/MeOH=9/1) to afford the tert-butyl 4-(4-(2-amino-4-(2-fluoro-4-(2-(4-fluorophenyl)-3-oxo-2,3-dihydropyridazine-4-carboxamido)phenoxy)pyridin-3-yl)-2-methylbut-3-yn-2-yl)piperazine-1-carboxylate (110 mg, 45%) as a pale yellow solid. ¹H-NMR (CDCl₃, Varian, 400 MHz): δ 1.43 (9H, s), 1.47 (6H, s), 2.61-2.64 (4H, m), 3.43-3.45 (4H, m), 5.07 (2H, s), 6.04 (1H, s), 7.10 (1H, t, J=8.8 Hz), 7.22-7.26 (2H, m), 7.32 (1H, d, J=9.2 Hz), 7.59-7.61 (2H, m), 7.82 (1H, brs), 7.89 (1H, dd, J=12.2 Hz), 8.22 (1H, d, J=4.4 Hz), 8.40 (1H, d, J=4.0 Hz).

### Step B: N-(4-(2-Amino-3-(3-methyl-3-(piperazin-1-yl)but-1-ynyl)pyridin-4-yloxy)-3-fluorophenyl)-2-(4-fluorophenyl)-3-oxo-2,3-dihydropyridazine-4-carboxamide

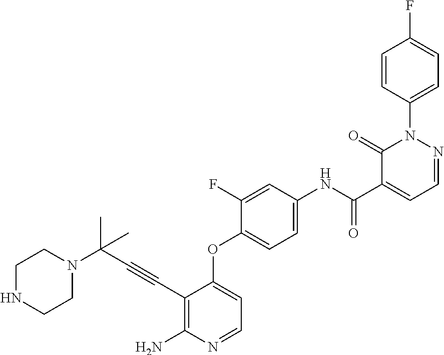 [SMILES: CC(C)(C#Cc1c(Oc2ccc(NC(=O)c3ccnn(-c4ccc(F)cc4)c3=O)cc2F)ccnc1N)N1CCNCC1]

To a solution of tert-butyl 4-(4-(2-amino-4-(2-fluoro-4-(2-(4-fluorophenyl)-3-oxo-2,3-dihydropyridazine-4-carboxamido)phenoxy)pyridin-3-yl)-2-methylbut-3-yn-2-yl)piperazine-1-carboxylate (0.11 g, 0.16 mmol) in DCM (4.0 mL) was added TFA (0.12 mL, 1.60 mmol) at room temperature. The reaction mixture was stirred for 18 h at room temperature. The reaction mixture was basified with saturated NaHCO₃(aq.) and extracted with EtOAc. The organic layer was dried over Na₂SO₄, filtered, and concentrated in vacuo. The residue was purified by column chromatography on SiO₂ (EtOAc/MeOH=95/5) afford the N-(4-(2-amino-3-(3-methyl-3-(piperazin-1-yl)but-1-ynyl)pyridin-4-yloxy)-3-fluorophenyl)-2-(4-fluorophenyl)-3-oxo-2,3-dihydropyridazine-4-carboxamide (72 mg, 77%) as a yellow solid. ¹H-NMR (CDCl₃, Varian, 400 MHz): δ1.45 (6H, s), 2.63 (4H, s), 2.90-2.92 (4H, m), 5.01 (2H, s), 6.06 (1H, d, J=6.0 Hz), 7.09 (1H, t, J=8.8 Hz), 7.22-7.24 (2H, m), 7.32 (1H, d, J=8.4 Hz), 7.57-7.61 (2H, m), 7.84 (1H, d, J=6.0 Hz), 7.89 (2H, dd, J=12.2 Hz), 8.22 (1H, d, J=4.0 Hz), 8.40 (1H, d, J=4.4 Hz), 11.75 (1H, s).

### Example 23

This example describes the synthesis of N-(4-(2-amino-3-(3-(4-(2-methoxyethyl)piperazin-1-yl)-3-methylbut-1-ynyl)pyridine-4-yloxy)-3-fluorophenyl)-2-(4-fluorophenyl)-3-oxo-2,3-dihydropyridazine-4-carboxamide in an aspect of the invention. See FIG. 22.

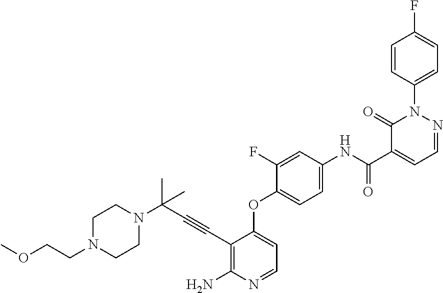 [SMILES: COCCN1CCN(C(C)(C)C#Cc2c(Oc3ccc(NC(=O)c4ccnn(-c5ccc(F)cc5)c4=O)cc3F)ccnc2N)CC1]

A mixture of N-(4-(2-amino-3-(3-methyl-3-(piperazin-1-yl)but-1-ynyl)pyridin-4-yloxy)-3-fluorophenyl)-2-(4-fluorophenyl)-3-oxo-2,3-dihydropyridazine-4-carboxamide (Example 22, 40 mg, 0.07 mmol), 1-bromo-2-methoxyethane (7.65 μL, 0.08 mmol), potassium iodide (11.3 mg, 0.07 mmol), and K₂CO₃ (9.44 mg, 0.07 mmol) in CH₃CN (2.0 mL) was heated for 5 h at 90° C. After being cooled at room temperature, the reaction mixture was washed with saturated NaHCO₃(aq.) and extracted with EtOAc. The organic layer was dried over Na₂SO₄, filtered, and concentrated in vacuo. The residue was purified by column chromatography on NH—SiO₂ (EtOAc to EtOAc/MeOH=95/5) to afford the N-(4-(2-amino-3-(3-(4-(2-methoxyethyl)piperazin-1-yl)-3-methylbut-1-ynyl)pyridine-4-yloxy)-3-fluorophenyl)-2-(4-fluorophenyl)-3-oxo-2,3-dihydropyridazine-4-carboxamide (30 mg, 68%) as a white solid. ¹H-NMR (CDCl₃, Varian, 400 MHz): δ 1.45 (6H, s), 2.54-2.57 (6H, m), 2.74 (4H, brs), 3.33 (3H, s), 3.49 (2H, t, J=6.0 Hz), 5.02 (2H, s), 6.03 (1H, d, J=5.6 Hz), 7.11 (1H, t, J=8.8 Hz), 7.23-7.24 (2H, m), 7.31 (1H, d, J=9.2 Hz), 7.57-7.61 (2H, m), 7.83 (1H, d, J=6.0 Hz), 7.88 (1H, dd, J=12.4 Hz), 8.22 (1H, d, J=4.4 Hz), 8.39 (1H, d, J=4.4 Hz), 11.75 (1H, s).

### Example 24

This example describes the synthesis of N-(4-(2-amino-3-(3-(4-(2-methoxyethyl)piperazin-1-yl)prop-2-ynyl)pyridine-4-yloxy)-3-fluorophenyl)-3-oxo-2,3-dihydropyridazine-4-carboxamide in an aspect of the invention. See FIG. 23.

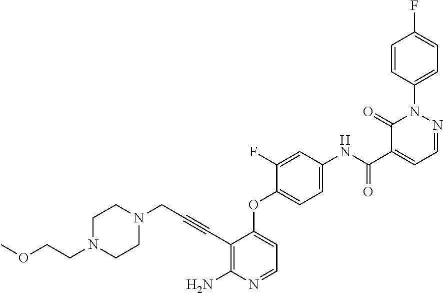 [SMILES: COCCN1CCN(CC#Cc2c(Oc3ccc(NC(=O)c4ccnn(-c5ccc(F)cc5)c4=O)cc3F)ccnc2N)CC1]

A mixture of N-(4-(2-amino-3-(3-(piperazin-1-yl)prop-1-ynyl)pyridin-4-yloxy)-3-fluorophenyl)-2-(4-fluorophenyl)-3-oxo-2,3-dihydropyridazine-4-carboxamide (Example 6, 40 mg, 0.07 mmol), 1-bromo-2-methoxyethane (8.03 μL, 0.08 mmol), potassium iodide (11.9 mg, 0.07 mmol), and K₂CO₃ (9.92 mg, 0.07 mmol) in CH₃CN (2.0 mL) was heated for 5 h at 90° C. After being cooled at room temperature, the reaction mixture was washed with saturated NaHCO₃(aq.) and extracted with EtOAc. The organic layer was dried over Na₂SO₄, filtered and concentrated in vacuo. The residue was purified by column chromatography on NH—SiO₂ (EtOAc/MeOH=95/5) to afford the N-(4-(2-amino-3-(3-(4-(2-methoxyethyl)piperazin-1-yl)prop-2-ynyl)pyridine-4-yloxy)-3-fluorophenyl)-3-oxo-2,3-dihydropyridazine-4-carboxamide (29 mg, 65%) as a white solid. ¹H-NMR (CDCl₃, Varian, 400 MHz): δ 2.57-2.69 (10H, m), 3.34 (3H, s), 3.49-3.51 (2H, m), 3.59 (2H, s), 5.09 (2H, s), 5.97 (1H, d, J=6.0 Hz), 7.14 (1H, t, J=8.8 Hz), 7.16-7.24 (2H, m), 7.33 (1H, d, J=4.8 Hz), 7.58-7.61 (2H, m), 7.82 (1H, d, J=5.6 Hz), 7.89 (1H, d, J=12.0 Hz), 8.22 (1H, d, J=4.0 Hz), 8.40 (1H, d, J=4.0 Hz), 11.77 (1H, s).

### Example 25

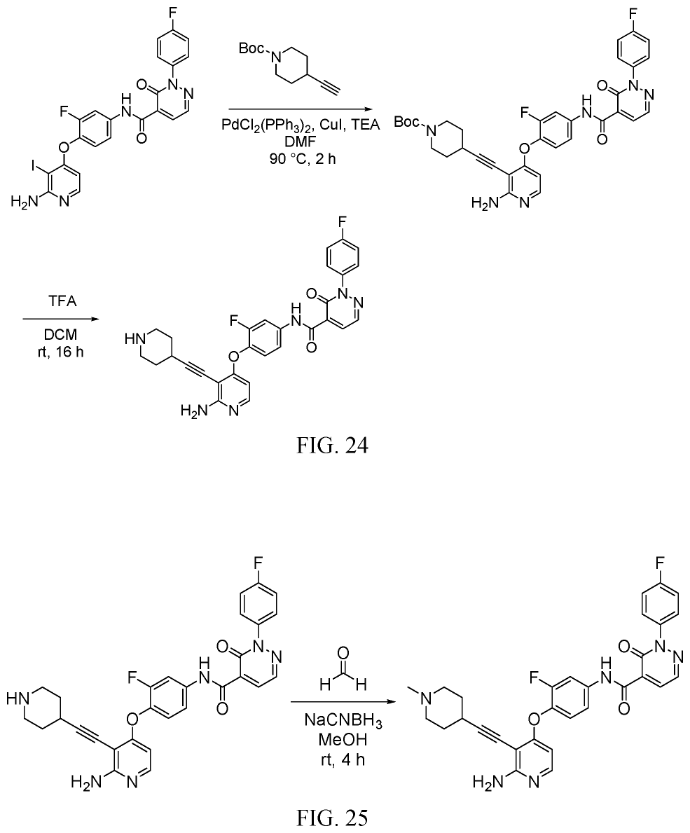

This example describes the synthesis of N-(4-(2-amino-3-(piperidin-4-ylethynyl)pyridine-4-yloxy)-3-fluorophenyl)-2-(4-fluorophenyl)-3-oxo-2,3-dihydropyridazine-4-carboxamide in an aspect of the invention. See FIG. 24.

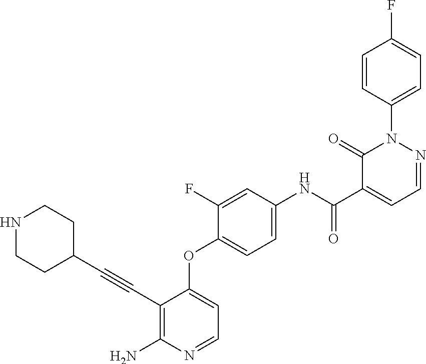 [SMILES: Nc1nccc(Oc2ccc(NC(=O)c3ccnn(-c4ccc(F)cc4)c3=O)cc2F)c1C#CC1CCNCC1]

### Step A: tert-Butyl 4-((2-amino-4-(2-fluoro-4-(2-(4-fluorophenyl)-3-oxo-2,3-dihydropyridazine-4-carboxamido)phenoxy)pyridin-3-yl)ethynyl)piperidine-1-carboxylate

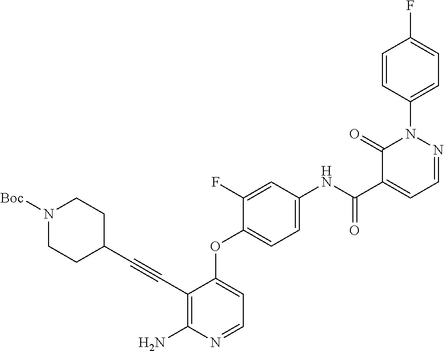 [SMILES: CC(C)(C)OC(=O)N1CCC(C#Cc2c(Oc3ccc(NC(=O)c4ccnn(-c5ccc(F)cc5)c4=O)cc3F)ccnc2N)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 200 mg, 0.36 mmol), tert-butyl 4-ethynylpiperidine-1-carboxylate (112 mg, 0.53 mmol), Pd(PPh₃)₄ (41.0 mg, 0.04 mmol), and copper (I) iodide (14.0 mg, 0.07 mmol) in DMF (3.0 mL) was purged with N₂. The reaction mixture was stirred for 2 h at 90° C. After being cooled at room temperature, EtOAc and saturated NH₄C₁ (aq.) were poured into the mixture, and the separated aqueous layer was extracted with EtOAc. The combined organic layer was dried over Na₂SO₄, filtered, and concentrated in vacuo. The residue was purified by MPLC (EtOAc/Hex=9/1) to afford the tert-butyl 4-((2-amino-4-(2-fluoro-4-(2-(4-fluorophenyl)-3-oxo-2,3-dihydropyridazine-4-carboxamido)phenoxy)pyridin-3-yl)ethynyl)piperidine-1-carboxylate (180 mg, 79%) as a pale yellow solid. ¹H-NMR (CD₃OD, Varian, 400 MHz): δ 1.35 (9H, s), 1.50-1.59 (2H, m), 1.60-1.79 (2H, m), 3.11-3.15 (2H, m), 3.60-3.64 (2H, m), 5.89 (1H, d, J=7.6 Hz), 7.03 (1H, t, J=8.8 Hz), 7.13 (2H, t, J=8.4 Hz), 7.23 (1H, d, J=8.8 Hz), 7.36-7.39 (3H, m), 7.44-7.56 (2H, m), 7.66 (1H, d, J=5.6 Hz), 7.80 (1H, d, J=11.6 Hz), 8.14 (1H, d, J=4.0 Hz), 8.28 (1H, d, J=4.0 Hz), 11.71 (1H, s).

### Step B: N-(4-(2-Amino-3-(piperidin-4-ylethynyl)pyridine-4-yloxy)-3-fluorophenyl)-2-(4-fluorophenyl)-3-oxo-2,3-dihydropyridazine-4-carboxamide

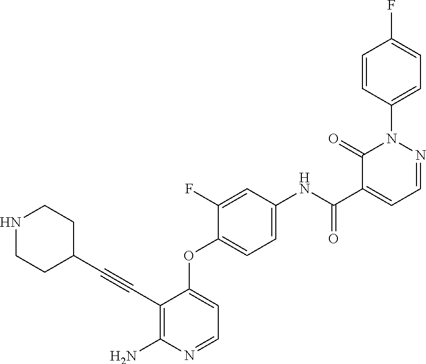 [SMILES: Nc1nccc(Oc2ccc(NC(=O)c3ccnn(-c4ccc(F)cc4)c3=O)cc2F)c1C#CC1CCNCC1]

To a solution of tert-butyl 4-((2-amino-4-(2-fluoro-4-(2-(4-fluorophenyl)-3-oxo-2,3-dihydropyridazine-4-carboxamido)phenoxy)pyridin-3-yl)ethynyl)piperidine-1-carboxylate (0.18 g, 0.28 mmol) in DCM (4.0 mL) was added TFA (0.21 mL, 2.80 mmol) at room temperature. The reaction mixture was stirred for 16 h at room temperature. The reaction mixture was basified with saturated NaHCO₃(aq.) and extracted with EtOAc. The organic layer was dried over Na₂SO₄, filtered, and concentrated in vacuo. The residue was purified by column chromatography on NH—SiO₂ (DCM/MeOH=97/3) afford the N-(4-(2-amino-3-(piperidin-4-ylethynyl)pyridine-4-yloxy)-3-fluorophenyl)-2-(4-fluorophenyl)-3-oxo-2,3-dihydropyridazine-4-carboxamide (0.14 g, 92%) as a yellow solid. ¹H-NMR (CDCl₃, Varian, 400 MHz): δ 1.61-1.72 (2H, m), 1.86-1.94 (2H, m), 2.69-2.75 (2H, m), 2.81-2.85 (1H, m), 3.06-3.11 (2H, m), 5.04 (2H, s), 6.01 (1H, d, J=6.0 Hz), 7.12 (1H, t, J=8.4 Hz), 7.22-7.24 (2H, m), 7.32 (1H, d, J=9.2 Hz), 7.57-7.61 (2H, m), 7.81 (1H, d, J=6.0 Hz), 7.88 (1H, dd, J=12.2 Hz), 8.22 (1H, d, J=4.4 Hz), 8.39 (1H, d, J=4.4 Hz), 11.76 (1H, s).

### Example 26

This example describes the synthesis of N-(4-(2-amino-3-((1-methylpiperidin-4-yl)ethynyl)pyridin-4-yloxy)-3-fluorophenyl)-2-(4-fluorophenyl)-3-oxo-2,3-dihydropyridazine-4-carboxamide in an aspect of the invention. See FIG. 25.

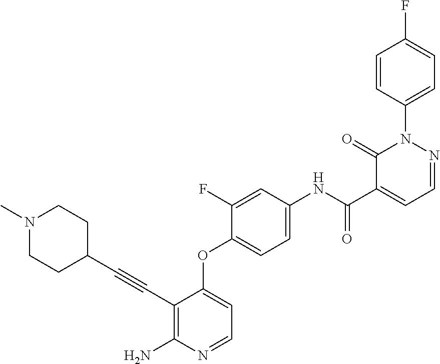 [SMILES: CN1CCC(C#Cc2c(Oc3ccc(NC(=O)c4ccnn(-c5ccc(F)cc5)c4=O)cc3F)ccnc2N)CC1]

A mixture of N-(4-(2-amino-3-(piperidin-4-ylethynyl)pyridine-4-yloxy)-3-fluorophenyl)-2-(4-fluorophenyl)-3-oxo-2,3-dihydropyridazine-4-carboxamide (Example 25, 30.0 mg, 0.05 mmol) and formaldehyde (21 μL, 0.28 mmol, 37%) in MeOH (5.0 mL) was stirred for 30 min at room temperature. After stirring, NaCNBH₃ (35.0 mg, 0.05 mmol) was added to the mixture and stirred for 4 h at room temperature. The reaction was quenched by saturated NaHCO₃(aq.) and diluted with DCM. The separated aqueous layer was extracted with DCM, the combined organic layer was dried over Na₂SO₄, filtered, and concentrated in vacuo. The residue was purified by column chromatography on NH—SiO₂ (EtOAc) to afford the N-(4-(2-amino-3-((1-methylpiperidin-4-yl)ethynyl)pyridin-4-yloxy)-3-fluorophenyl)-2-(4-fluorophenyl)-3-oxo-2,3-dihydropyridazine-4-carboxamide (12.0 mg, 39%) as a yellow solid. ¹H-NMR (CDCl₃, Varian, 400 MHz): δ 1.77-1.81 (2H, m), 1.91-1.95 (4H, m), 2.25 (3H, s), 2.67 (3H, m), 5.03 (2H, s), 6.00 (1H, d, J=6.0 Hz), 7.12 (1H, t, J=8.0 Hz), 7.23 (2H, d, J=8.8 Hz), 7.32 (1H, d, J=8.8 Hz), 7.57-7.61 (2H, m), 7.81 (1H, d, J=6.0 Hz), 7.89 (1H, dd, J=12.2 Hz), 8.22 (1H, d, J=4.4 Hz), 8.40 (1H, d, J=4.0 Hz), 11.76 (1H, s).

### Example 27

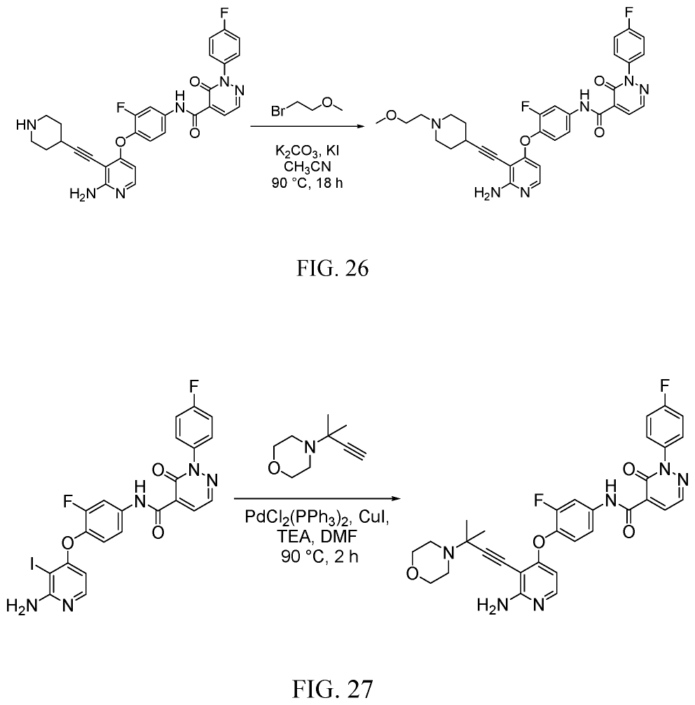

This example describes the synthesis of N-(4-(2-amino-3-((1-(2-methoxyethyl)piperidin-4-yl)ethynyl)pyridin-4-yloxy)-3-fluorophenyl)-2-(4-fluorophenyl)-3-oxo-2,3-dihydropyridazine-4-carboxamide in an aspect of the invention. See FIG. 26.

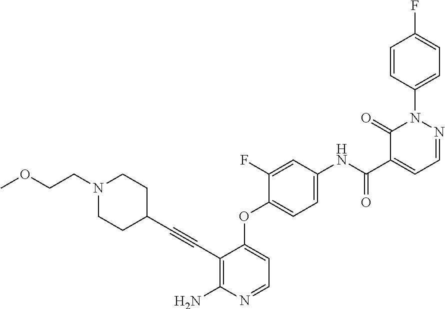 [SMILES: COCCN1CCC(C#Cc2c(Oc3ccc(NC(=O)c4ccnn(-c5ccc(F)cc5)c4=O)cc3F)ccnc2N)CC1]

A mixture of N-(4-(2-amino-3-(piperidin-4-ylethynyl)pyridine-4-yloxy)-3-fluorophenyl)-2-(4-fluorophenyl)-3-oxo-2,3-dihydropyridazine-4-carboxamide (Example 25, 140 mg, 0.26 mmol), 1-bromo-2-methoxyethane (29 μL, 0.31 mmol), potassium iodide (43 mg, 26 mmol), and K₂CO₃ (36 mg, 0.26 mmol) in CH₃CN (5.0 mL) was heated to 90° C. for 18 h. After being cooled to room temperature, the reaction mixture was washed with saturated NaHCO₃(aq.) and extracted with EtOAc. The organic layer was dried over Na₂SO₄, filtered, and concentrated in vacuo. The residue was purified by column chromatography on NH—SiO₂ (EtOAc/MeOH=95/5) to afford the N-(4-(2-amino-3-((1-(2-methoxyethyl)piperidin-4-yl)ethynyl)pyridin-4-yloxy)-3-fluorophenyl)-2-(4-fluorophenyl)-3-oxo-2,3-dihydropyridazine-4-carboxamide (67 mg, 43%) as a yellow solid. ¹H-NMR (CDCl₃, Varian, 400 MHz): δ 1.75-1.83 (2H, m), 1.96-2.16 (2H, m), 2.32 (2H, m), 2.54-2.57 (2H, m), 2.78-2.83 (3H, m), 3.29 (3H, s), 3.48-3.51 (2H, m), 5.03 (2H, m), 5.99 (1H, d, J=6.0 Hz), 7.11 (1H, t, J=8.8 Hz), 7.21-7.24 (2H, m), 7.32 (1H, d, J=8.4 Hz), 7.57-7.60 (2H, m), 7.80 (1H, d, J=6.4 Hz), 7.88 (1H, dd, J=12.0 Hz), 8.22 (1H, d, J=4.0 Hz), 8.39 (1H, d, J=4.4 Hz), 11.75 (1H, s).

### Example 28

This example describes the synthesis of N-(4-(2-amino-3-(3-methyl-3-morpholinobut-1-ynl)pyridin-4-yloxy)-3-fluorophenyl)-2-(4-fluorophenyl)-3-oxo-2,3-dihydropyridazine-4-carboxamide in an aspect of the invention. See FIG. 27.

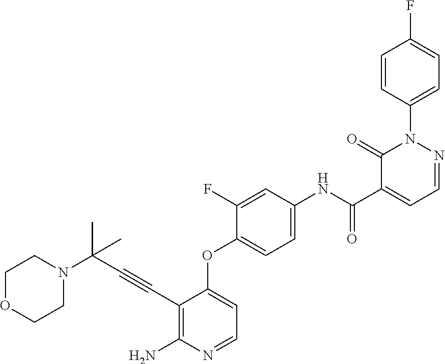 [SMILES: CC(C)(C#Cc1c(Oc2ccc(NC(=O)c3ccnn(-c4ccc(F)cc4)c3=O)cc2F)ccnc1N)N1CCOCC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 50 mg, 0.09 mmol), 4-(2-methylbut-3-yn-2-yl)morpholine (intermediate 13, 14 mg, 0.09 mmol), Pd(PPh₃)₄ (10 mg, 8.91 μmol) and copper (I) iodide (4.0 mg, 0.02 mmol) in DMF (1.0 mL) was purged with N₂. The reaction mixture was stirred for 2 h at 90° C. After cooled at room temperature, EtOAc and saturated NH₄C₁ (aq.) were poured into the residue, and the separated aqueous layer was extracted with EtOAc. The combined organic layer was dried over Na₂SO₄, filtered, and concentrated in vacuo. The residue was purified by MPLC (Hex/EtOAc=1/9) to afford the N-(4-(2-amino-3-(3-methyl-3-morpholinobut-1-ynl)pyridin-4-yloxy)-3-fluorophenyl)-2-(4-fluorophenyl)-3-oxo-2,3-dihydropyridazine-4-carboxamide (10 mg, 19%) as a pale yellow solid. ¹H-NMR (CDCl₃, Varian, 400 MHz): δ 1.25 (6H, s), 2.64-2.69 (4H, m), 3.72-3.74 (4H, m), 5.10 (2H, s), 6.05 (1H, d, J=5.6 Hz), 7.10 (1H, t, J=8.4 Hz), 7.23 (2H, d, J=8.4 Hz), 7.32 (1H, d, J=9.2 Hz), 7.57-7.61 (2H, m), 7.84 (1H, brs), 7.89 (1H, dd, J=12.4 Hz), 8.22 (1H, d, J=4.0 Hz), 8.39 (1H, d, J=4.0 Hz), 11.76 (1H, s).

### Example 29

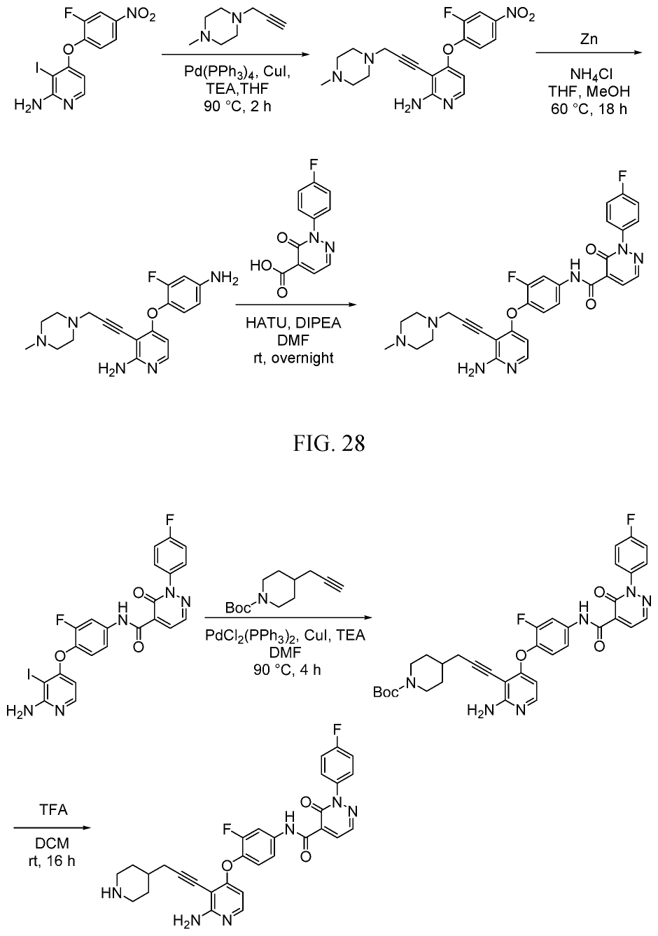

This example describes the synthesis of N-(4-(2-amino-3-(3-(4-methylpiperazin-1-yl)prop-1-ynyl)pyridin-4-yloxy)-3-fluorophenyl)-2-(4-fluorophenyl)-3-oxo-2,3-dihydropyridazine-4-carboxamide in an aspect of the invention. See FIG. 28.

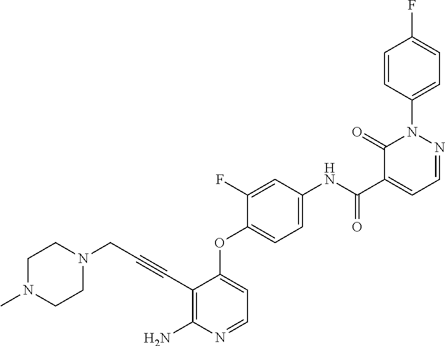 [SMILES: CN1CCN(CC#Cc2c(Oc3ccc(NC(=O)c4ccnn(-c5ccc(F)cc5)c4=O)cc3F)ccnc2N)CC1]

### Step A: 4-(2-Fluoro-4-nitrophenoxy)-3-(3-(4-methylpiperazin-1-yl)prop-1-ynyl)pyridin-2-amine

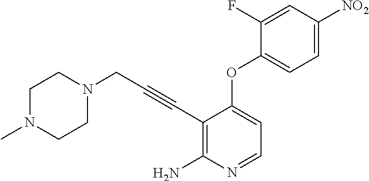 [SMILES: CN1CCN(CC#Cc2c(Oc3ccc([N+](=O)[O-])cc3F)ccnc2N)CC1]

A mixture of 4-(2-fluoro-4-nitrophenoxy)-3-iodopyridin-2-amine (step D of intermediate 19, 200 mg, 0.53 mmol), 1-methyl-4-(prop-2-ynyl)piperazine (intermediate 14, 110 mL, 0.80 mmol), Pd(PPh₃)₄ (62 mg, 53.0 μmol), and copper(I) iodide (20.0 mg, 0.10 mmol) in DMF (2.0 mL) was purged with N₂. The reaction mixture was stirred for 2 h at 90° C. After cooled to room temperature, EtOAc and saturated NH₄C₁ (aq.) were poured into the residue, and the separated aqueous layer was extracted with EtOAc. The combined organic layer was dried over Na₂SO₄, filtered, and concentrated in vacuo. The residue was purified by MPLC (EtOAc/MeOH) to afford the 4-(2-fluoro-4-nitrophenoxy)-3-(3-(4-methylpiperazin-1-yl)prop-1-ynyl)pyridin-2-amine (139 mg, 67%) as a yellow solid. ¹H-NMR (CDCl₃, Varian, 400 MHz): δ 2.68 (3H, s), 2.29-2.53 (8H, m), 3.51 (2H, s), 5.28 (2H, s), 6.20 (1H, d, J=6.0 Hz), 7.37 (1H, t, J=8.0 Hz), 8.04 (1H, d, J=3.6 Hz), 8.05-8.06 (1H, m), 8.10 (1H, d, J=11.6 Hz).

### Step B: 4-(4-Amino-2-fluorophenyl)-3-(3-(4-methylpiperazin-1-yl)prop-1-ynyl)pyridin-2-amine

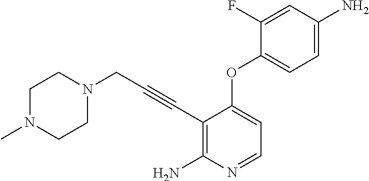 [SMILES: CN1CCN(CC#Cc2c(Oc3ccc(N)cc3F)ccnc2N)CC1]

A mixture of 4-(2-fluoro-4-nitrophenoxy)-3-(3-(4-methylpiperazin-1-yl)prop-1-ynyl)pyridin-2-amine (130 mg, 0.36 mmol), zinc (236 mg, 3.61 mmol), and NH₄C₁ (193 mg, 3.61 mmol) in THF-MeOH (v/v=1/1, 6 mL) was stirred for 18 h at 60° C. After being cooled at room temperature, the solvent was evaporated in vacuo and the residue was dissolved with EtOAc. The organic layer was washed with saturated NaHCO₃(aq.) and dried over Na₂SO₄, filtered, and concentrated in vacuo. The residue was purified by MPLC (DCM/MeOH) to afford the 4-(4-Amino-2-fluorophenyl)-3-(3-(4-methylpiperazin-1-yl)prop-1-ynyl)pyridin-2-amine (49.0 mg, 39%) as a yellow solid. ¹H-NMR (CDCl₃, Varian, 400 MHz): δ 2.29 (3H, s), 2.52 (4H, brs), 2.70 (4H, brs), 3.62 (2H, s), 5.09 (2H, s), 5.94 (1H, d, J=6.0 Hz), 6.41-6.44 (1H, m), 6.49 (1H, dd, J=11.8 Hz), 6.94 (1H, t, J=8.4 Hz), 7.78 (1H, d, J=5.6 Hz). * NH₂ peak was not observed.

### Step C: N-(4-(2-Amino-3-(3-(4-methylpiperazin-1-yl)prop-1-ynyl)pyridin-4-yloxy)-3-fluorophenyl)-2-(4-fluorophenyl)-3-oxo-2,3-dihydropyridazine-4-carboxamide

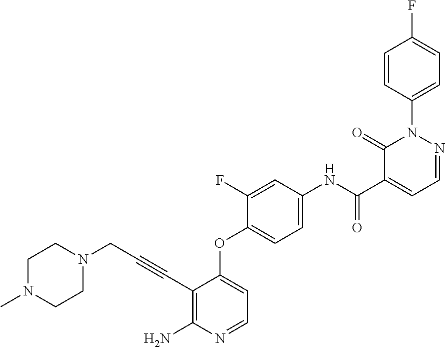 [SMILES: CN1CCN(CC#Cc2c(Oc3ccc(NC(=O)c4ccnn(-c5ccc(F)cc5)c4=O)cc3F)ccnc2N)CC1]

A mixture of 4-(4-amino-2-fluorophenyl)-3-(3-(4-methylpiperazin-1-yl)prop-1-ynyl)pyridin-2-amine (49.0 mg, 0.14 mmol), 2-(4-fluorophenyl)-3-oxo-2,3-dihydropyridazine-4-carboxylic acid (intermediate 15, 32.0 mg, 0.14 mmol), HATU (58.0 mg, 0.15 mmol) and DIPEA (60.0 μL, 0.34 mmol) in DMF (3.0 mL) was stirred for 1 h at 50° C. After cooled to room temperature, EtOAc and saturated NH₄C₁ (aq.) were poured into the residue, and the separated aqueous layer was extracted with EtOAc. The combined organic layer was dried over Na₂SO₄, filtered, and concentrated in vacuo. The residue was purified by column chromatography on NH—SiO₂ (DCM/MeOH=100/1) to afford the N-(4-(2-Amino-3-(3-(4-methylpiperazin-1-yl)prop-1-ynyl)pyridin-4-yloxy)-3-fluorophenyl)-2-(4-fluorophenyl)-3-oxo-2,3-dihydropyridazine-4-carboxamide (25 mg, 31%) as a yellow solid. ¹H-NMR (CDCl₃, Varian, 400 MHz): δ 2.29 (3H, s), 2.50 (4H, brs), 2.68 (4H, brs), 3.60 (2H, s), 5.08 (2H, s), 5.97 (1H, d, J=6.0 Hz), 7.13 (1H, t, J=8.8 Hz), 7.22-7.26 (2H, m), 7.33 (1H, d, J=8.0 Hz), 7.57-7.61 (2H, m), 7.82 (1H, d, J=5.6 Hz), 7.89 (1H, dd, J=12.4 Hz), 8.22 (1H, d, J=4.4 Hz), 8.40 (1H, d, J=4.4 Hz), 11.77 (1H, s).

### Example 30

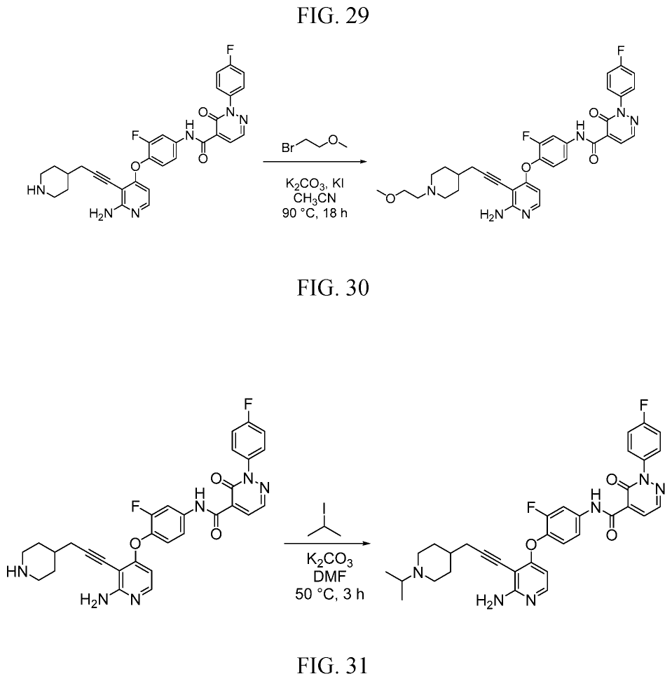

This example describes the synthesis of N-(4-(2-amino-3-(3-(piperidin-4-yl)prop-1-ynyl)pyridin-4-yloxy)-3-fluorophenyl)-2-(4-fluorophenyl)-3-oxo-2,3-dihydropyridazine-4-carboxamide in an aspect of the invention. See FIG. 29.

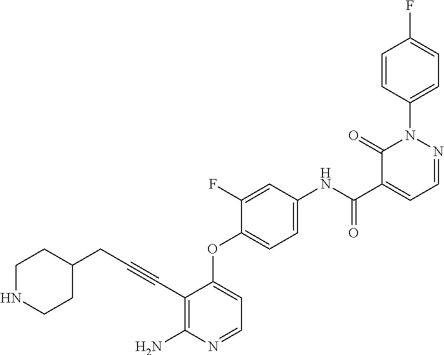 [SMILES: Nc1nccc(Oc2ccc(NC(=O)c3ccnn(-c4ccc(F)cc4)c3=O)cc2F)c1C#CCC1CCNCC1]

### Step A: tert-Butyl 4-(3-(2-amino-4-(2-fluoro-4-(2-(4-fluorophenyl)-3-oxo-2,3-dihydropyridazine-4-carboxamido)phenoxy)pyridine-3-yl)prop-2-ynyl)piperidine-1-carboxylate

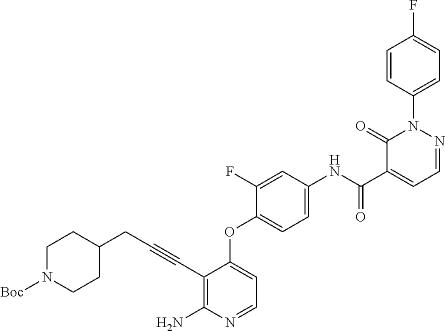 [SMILES: CC(C)(C)OC(=O)N1CCC(CC#Cc2c(Oc3ccc(NC(=O)c4ccnn(-c5ccc(F)cc5)c4=O)cc3F)ccnc2N)CC1]

A mixture of N-(4-(2-amino-3-iodopyridin-4-yloxy)-3-fluorophenyl)-2-(4-fluorophenyl)-3-oxo-2,3-dihydropyridazine-4-carboxamide (intermediate 19, 200 mg, 0.36 mmol), tert-butyl 4-(prop-2-ynyl)piperidine-1-carboxylate (0.12 mg, 0.53 mmol), Pd(PPh₃)₄ (41 mg, 0.04 mmol), and copper(I) iodide (14 mg, 0.07 mmol) in DMF (3.0 mL) was purged with N₂. The reaction mixture was stirred for 4 h at 90° C. After cooled to room temperature, EtOAc and saturated NH₄Cl (aq.) were poured into the residue, and the separated aqueous layer was extracted with EtOAc. The combined organic layers were dried over Na₂SO₄, filtered, and concentrated in vacuo. The residue was purified by MPLC (Hex/EtOAc=1/4) to afford the tert-butyl 4-(3-(2-amino-4-(2-fluoro-4-(2-(4-fluorophenyl)-3-oxo-2,3-dihydropyridazine-4-carboxamido)phenoxy)pyridine-3-yl)prop-2-ynyl)piperidine-1-carboxylate (150 mg, 64%) as a brown oil. ¹H-NMR (MeOD, Varian, 400 MHz): δ 1.44-1.60 (10H, m), 1.71-1.71 (2H, brs), 1.81 (2H, d, J=13.2 Hz), 2.45 (2H, d, J=6.4 Hz), 2.69 (2H, brs), 5.14 (2H, s), 5.99 (1H, d, J=5.2 Hz), 7.12 (1H, t, J=8.4 Hz), 7.22-7.27 (3H, m), 7.32 (1H, d, J=9.2 Hz), 7.65-7.69 (2H, m), 7.80 (1H, brs), 7.78-7.91 (1H, m), 8.23 (1H, d, J=4.0 Hz), 8.40 (1H, d, J=4.0 Hz), 11.7 (1H, s). *NH peak was not observed.

### Step B: N-(4-(2-Amino-3-(3-(piperidin-4-yl)prop-1-ynyl)pyridin-4-yloxy)-3-fluorophenyl)-2-(4-fluorophenyl)-3-oxo-2,3-dihydropyridazine-4-carboxamide

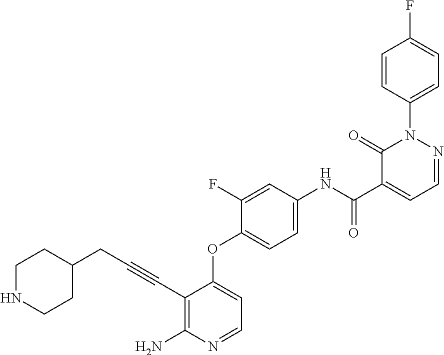 [SMILES: Nc1nccc(Oc2ccc(NC(=O)c3ccnn(-c4ccc(F)cc4)c3=O)cc2F)c1C#CCC1CCNCC1]

To a solution of tert-butyl 4-(3-(2-amino-4-(2-fluoro-4-(2-(4-fluorophenyl)-3-oxo-2,3-dihydropyridazine-4-carboxamido)phenoxy)pyridine-3-yl)prop-2-ynyl)piperidine-1-carboxylate (0.17 g, 0.26 mmol) in DCM (6.0 mL) was added TFA (0.20 mL, 2.60 mmol) at room temperature. The reaction mixture was stirred for 16 h at room temperature. The reaction mixture was basified with saturated NaHCO₃(aq.) and extracted with EtOAc. The organic layer was dried over Na₂SO₄, filtered, and concentrated in vacuo. The residue was purified by column chromatography on SiO₂ (EtOAc/MeOH=95/5) afford the N-(4-(2-amino-3-(3-(piperidin-4-yl)prop-1-ynyl)pyridin-4-yloxy)-3-fluorophenyl)-2-(4-fluorophenyl)-3-oxo-2,3-dihydropyridazine-4-carboxamide (62 mg, 43%) as a yellow solid. ¹H-NMR (CDCl₃, Varian, 400 MHz): δ 1.68-1.71 (1H, m), 1.82 (2H, d, J=12.4 Hz), 2.43 (2H, d, J=6.8 Hz), 2.56-2.62 (2H, m), 3.08 (2H, d, J=12.0 Hz), 5.03 (2H, s), 6.00 (1H, d, J=6.0 Hz), 7.12 (1H, t, J=8.8 Hz), 7.22-7.26 (3H, m), 7.32 (1H, d, J=9.2 Hz), 7.57-7.61 (2H, m), 7.81 (1H, d, J=6.0 Hz), 7.89 (1H, dd, J=2.0 Hz, 12.2 Hz), 8.23 (1H, d, J=4.0 Hz), 8.40 (1H, d, J=4.4 Hz), 11.7 (1H, s). *NH₂ peak was not observed.

### Example 31

This example describes the synthesis of N-(4-(2-amino-3-(3-(1-(2-methoxyethyl)piperidin-4-yl)prop-1-ynyl)pyridin-4-yloxy)-3-fluorophenyl)-2-(4-fluorophenyl)-3-oxo-2,3-dihydropyridazine-4-carboxamide in an aspect of the invention. See FIG. 30.

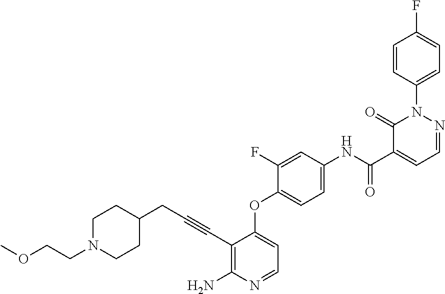 [SMILES: COCCN1CCC(CC#Cc2c(Oc3ccc(NC(=O)c4ccnn(-c5ccc(F)cc5)c4=O)cc3F)ccnc2N)CC1]

A mixture of N-(4-(2-amino-3-(3-(piperidin-4-yl)prop-1-ynyl)pyridin-4-yloxy)-3-fluorophenyl)-2-(4-fluorophenyl)-3-oxo-2,3-dihydropyridazine-4-carboxamide (Example 30, 30 mg, 0.05 mmol), 1-bromo-2-methoxyethane (6.03 μL, 0.06 mmol), potassium iodide (8.9 mg, 0.05 mmol), and K₂CO₃ (7.4 mg, 0.05 mmol) in CH₃CN (1.0 mL) was heated for 18 h at 90° C. After being cooled at room temperature, the reaction mixture was washed with saturated NaHCO₃(aq.) and extracted with EtOAc. The organic layer was dried over Na₂SO₄, filtered and concentrated in vacuo. The residue was purified by column chromatography on NH—SiO₂ (EtOAc/MeOH=95/5) to afford the N-(4-(2-amino-3-(3-(1-(2-methoxyethyl)piperidin-4-yl)prop-1-ynyl)pyridin-4-yloxy)-3-fluorophenyl)-2-(4-fluorophenyl)-3-oxo-2,3-dihydropyridazine-4-carboxamide (15 mg, 45%) as a yellow solid. ¹H-NMR (CDCl₃, Varian, 400 MHz): δ 1.82 (3H, d, J=12.8 Hz), 1.99 (2H, t, J=10.4 Hz), 2.43 (2H, d, J=7.2 Hz), 2.55 (2H, t, J=6.0 Hz), 2.96 (2H, d, J=11.2 Hz), 3.34 (3H, s), 3.50 (2H, t, J=5.2 Hz), 5.03 (2H, s), 5.99 (1H, d, J=5.6 Hz), 7.12 (1H, t, J=8.8 Hz), 7.22-7.24 (3H, m), 7.32 (2H, d, J=8.8 Hz), 7.57-7.61 (2H, m), 7.80 (1H, d, J=6.0 Hz), 7.89 (1H, dd, J=2.0 Hz, 12.2 Hz), 8.23 (1H, d, J=4.0 Hz), 8.40 (1H, d, J=4.4 Hz), 11.7 (1H, s).

### Example 32

This example describes the synthesis of N-(4-(2-amino-3-(3-(1-isopropylpiperidin-4-yl)prop-1-ynyl)pyridin-4-yloxy)-3-fluorophenyl)-2-(4-fluorophenyl)-3-oxo-2,3-dihydropyridazine-4-carboxamide in an aspect of the invention. See FIG. 31.

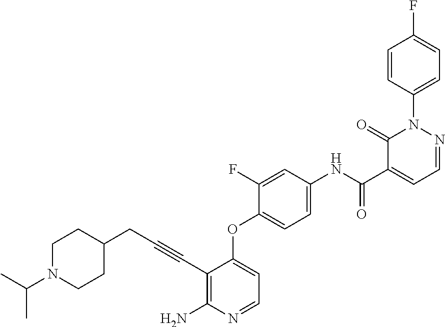 [SMILES: CC(C)N1CCC(CC#Cc2c(Oc3ccc(NC(=O)c4ccnn(-c5ccc(F)cc5)c4=O)cc3F)ccnc2N)CC1]

A mixture of the N-(4-(2-amino-3-(3-(piperidin-4-yl)prop-1-ynyl)pyridin-4-yloxy)-3-fluorophenyl)-2-(4-fluorophenyl)-3-oxo-2,3-dihydropyridazine-4-carboxamide (Example 30, 50 mg, 0.09 mmol), 2-iodopropane (0.02 mL, 0.18 mmol), and K₂CO₃ (25 mg, 0.18 mmol) in DMF (2.0 mL) was heated for 3 h at 50° C. After being cooled at room temperature, the reaction mixture was washed with water and extracted with EtOAc. The organic layer was dried over Na₂SO₄, filtered, and concentrated in vacuo. The residue was purified by column chromatography on NH—SiO₂ (DCM/MeOH=97/3) to afford the N-(4-(2-amino-3-(3-(1-isopropylpiperidin-4-yl)prop-1-ynyl)pyridin-4-yloxy)-3-fluorophenyl)-2-(4-fluorophenyl)-3-oxo-2,3-dihydropyridazine-4-carboxamide (12 mg, 22%) as a white solid. ¹H-NMR (MeOD, Varian, 400 MHz): δ 0.90 (6H, d, J=6.4 Hz), 1.15-1.21 (2H, m), 1.39-1.40 (1H, m), 1.66 (2H, d, J=12.0 Hz), 2.04 (2H, t, J=10.0 Hz), 2.40 (2H, d, J=12.0 Hz), 2.59-2.67 (1H, m), 2.70 (2H, d, J=12.0 Hz), 5.95 (1H, d, J=5.6 Hz), 6.17 (2H, s), 7.23 (1H, t, J=9.2 Hz), 7.41 (2H, t, J=8.4 Hz), 7.47 (1H, d, J=7.2 Hz), 7.65-7.68 (2H, m), 7.78 (1H, d, J=5.6 Hz), 7.96 (1H, dd, J=12.4 Hz, 2.4 Hz), 8.25 (1H, d, J=4.4 Hz), 8.37 (1H, d, J=4.0 Hz), 11.6 (1H, s).

### Example 33

This example illustrates an enzymatic assay to determine the inhibitory activity of exemplary compounds of Formula (I) in an aspect of the invention.

All the kinase reactions were performed in 5 μL using tyrosine kinase buffer with 0.2 μg/μL poly (Glu4, Tyr1) substrate, 10 μM ATP, serial dilution of the inhibitor, and incubated at room temperature for 60 min. After the indicated incubation times, 5 μL ADP-GLO™ reagent (Promega, Madison, Wis.) was added to the reactions and the plate was incubated at room temperature for 40 min. Then, 10 μL of kinase detection reagent was added and after an incubation time of 40 min, luminescence was recorded and IC₅₀ values were determined (Table 1). All 384-well assay plates were read using a GLOMAX™ Discover Microplate Luminometer from Promega (Madison, Wis.). To plot, analyze the data and calculate all kinase reaction biochemical values, both Microsoft Excel and Prism from GraphPad 7 Software (La Jolla, Calif.) were used.

TABLE 1

| Example | Enzymatic assay IC₅₀ (nM) | | |
|---|---|---|---|
| Number | C-MET | AXL | MER |
| 1 | <1000 | <1000 | <1000 |
| 2 | <1000 | <100 | <10 |
| 3 | <1000 | <1000 | <1000 |
| 4 | <100 | <1000 | <100 |
| 5 | <10 | <100 | <1000 |
| 6 | <1000 | <1000 | <1000 |
| 7 | <1000 | <1000 | <1000 |
| 8 | <1000 | <100 | <1000 |
| 9 | <100 | <10 | <100 |
| 10 | <1000 | <100 | <1000 |
| 11 | <10 | <100 | <1000 |
| 12 | <100 | <100 | <100 |
| 13 | <100 | <100 | <100 |
| 14 | <10 | <10 | <10 |
| 15 | <100 | <100 | <100 |
| 20 | >1000 | <1000 | <1000 |
| 21 | >1000 | <10 | <100 |
| 22 | <100 | <10 | <100 |
| 23 | <100 | <10 | <100 |
| 24 | <100 | <10 | <10 |
| 25 | <10 | <10 | >1000 |
| 26 | <10 | <10 | >1000 |
| 27 | <10 | <10 | <10 |
| 28 | <10 | <10 | <100 |
| 29 | <10 | <10 | <10 |
| 30 | <10 | <10 | <10 |
| 31 | <10 | <100 | <100 |
| 32 | <100 | <100 | <100 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of Formula (I) or a pharmaceutically acceptable salt 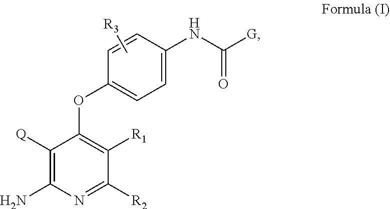
wherein
R¹ is H, alkyl, haloalkyl, halo, or CN;
R² is H, alkyl, haloalkyl, halo, or CN;
R³ is H or halo;
Q is H, CN, halo, alkyl, alkenyl, alkynyl, cycloalkyl, heterocyclyl, heteroaryl, or aryl, wherein said alkenyl or alkynyl is selected from the group consisting of
—CH═CR⁴(CX′)ₘ(CH₂)ₙNR⁵R⁶, —C≡C(CX′)ₘ(CH₂)ₙNR⁵R⁶,
—CH═CR⁴(CX′)ₘ(CH₂)ₙCHR⁵R⁶, —C≡C(CX′)ₘ(CH₂)ₙCHR⁵R⁶,
—CH═CR⁴(CX′)ₘ(CH₂)ₙNR⁷OR⁸, and —C≡C(CX′)ₘ(CH₂)ₙNR⁷OR⁸;
wherein
R⁴ is hydrogen or halo;
X′ is H₂, (C₁₋₆ alkyl)₂, or ═O;
m is 0 or 1;
n is 0 or 1-3;
—NR⁵R⁶ either forms a 4-7 membered heterocyclic ring or does not form a ring structure, the heterocyclic ring being either heteroaryl or heterocyclyl ring,
when —NR⁵R⁶ forms a 4-7 membered heterocyclic ring, the 4-7 membered heterocyclic ring includes an optional second heteroatom in addition to the nitrogen of —NR⁵R⁶ and is optionally substituted with one or more substituent groups independently selected from the group consisting of linear C₁-C₆ alkyl, branched C₃-C₆ alkyl, hydroxy, C₁-C₆ alkoxyalkyl, carboxylic acid, linear C₁-C₄ alkyl carboxylic acid, and branched C₃-C₄ alkyl carboxylic acid;
when —NR⁵R⁶ does not form a ring structure, R⁵ is selected from the group consisting of hydrogen, linear C₁-C₆ alkyl, and branched C₃-C₆ alkyl, and R⁶ is selected from the group consisting of hydrogen, linear C₁-C₆ alkyl optionally substituted with at least one fluoro or at least one hydroxy, branched C₃-C₆ alkyl optionally substituted with at least one fluoro or at least one hydroxy, and cycloalkyl optionally substituted with at least one fluoro or at least one hydroxy;
—CHR⁵R⁶ either forms a 4-7 membered heterocyclic ring or does not form a ring structure, the heterocyclic ring being either heteroaryl or heterocyclyl ring,
when —CHR⁵R⁶ forms a 4-7 membered heterocyclic ring, the 4-7 membered heterocyclic ring includes one or two heteroatoms and is optionally substituted with one or more substituent groups independently selected from the group consisting of linear C₁-C₆ alkyl, branched C₃-C₆ alkyl, hydroxy, C₁-C₆ alkoxyalkyl, carboxylic acid, linear C₁-C₄ alkyl carboxylic acid, and branched C₃-C₄ alkyl carboxylic acid;
when —CHR⁵R⁶ does not form a ring structure, R⁵ is selected from the group consisting of hydrogen, linear C₁-C₆ alkyl, and branched C₃-C₆ alkyl, and R⁶ is selected from the group consisting of hydrogen, linear C₁-C₆ alkyl optionally substituted with at least one fluoro or at least one hydroxy, branched C₃-C₆ alkyl optionally substituted with at least one fluoro or at least one hydroxy, and cycloalkyl optionally substituted with at least one fluoro or at least one hydroxy;
—NR⁷OR⁸ does not form a ring structure, R⁷ is selected from the group consisting of hydrogen, linear C₁-C₆ alkyl, and branched C₃-C₆ alkyl, and R⁸ is selected from the group consisting of hydrogen, linear C₁-C₆ alkyl optionally substituted with at least one fluoro, hydroxy, or alkoxy group, branched C₃-C₆ alkyl optionally substituted with at least one fluoro, hydroxy, or alkoxy group, and cycloalkyl optionally substituted with at least one fluoro, hydroxy, or alkoxy group;
G is 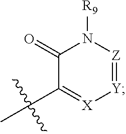
wherein
R⁹ is phenyl substituted with alkyl, haloalkyl, halo, and/or CN;
each X, Y, and Z is independently CR¹⁰ or N; and
R¹⁰ is H, C₁-C₆ alkyl, or C₁-C₆ alkoxy.

2. The compound of claim 1 or a pharmaceutically acceptable salt thereof, wherein both R¹ and R² are hydrogen.

3. The compound of claim 1 or a pharmaceutically acceptable salt thereof, wherein R³ is a halo.

4. The compound of claim 1 or a pharmaceutically acceptable salt thereof, wherein Q is CN, halo, optionally substituted phenyl, optionally substituted heterocyclyl, or an alkenyl or alkynyl moiety selected from the group consisting of —CH═CR⁴(CX′)ₘ(CH₂)ₙNR⁵R⁶, —C≡C(CX′)ₘ(CH₂)ₙNR⁵R⁶, —CH═CR⁴(CX′)ₘ(CH₂)ₙCHR⁵R⁶, —C≡C(CX′)ₘ(CH₂)ₙCHR⁵R⁶, —CH═CR⁴(CX′)ₘ(CH₂)ₙNR⁷OR⁸, and —C≡C(CX′)ₘ(CH₂)ₙNR⁷OR⁸,
wherein
R⁴ is hydrogen or halo;
X′ is H₂, (C₁₋₆ alkyl)₂, or ═O;
m is 0 or 1;
n is 0 or 1;
—NR⁵R⁶ is morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl,
—CHR⁵R⁶ is tetrahydropyranyl, morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl,
R⁷ is selected from the group consisting of hydrogen, linear C₁-C₆ alkyl, and branched C₃-C₆ alkyl, and
R⁸ is selected from the group consisting of linear C₁-C₆ alkyl optionally substituted with at least one alkoxy group and branched C₃-C₆ alkyl optionally substituted with at least one alkoxy group.

5. The compound of claim 1 or a pharmaceutically acceptable salt thereof, wherein
R⁹ is phenyl substituted with alkyl, haloalkyl, halo, and/or CN; and
either (i) X is N, and Y and Z are CH, (ii) X and Y are CH, and Z is N, or (iii) X, Y, and Z are each CR¹⁰.

6. The compound of claim 1 or a pharmaceutically acceptable salt thereof, wherein the compound of Formula (I) is a compound Formula (Ib): 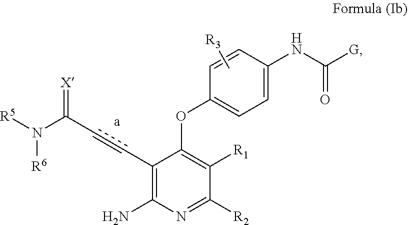
wherein [symbol] is —C≡C— or —CH═CH—.

7. The compound of claim 6 or a pharmaceutically acceptable salt thereof, wherein both R¹ and R² are hydrogen.

8. The compound of claim 6 or a pharmaceutically acceptable salt thereof, wherein R³ is a halo.

9. The compound of claim 6 or a pharmaceutically acceptable salt thereof, wherein
X′ is H₂, (C₁₋₆ alkyl)₂, or ═O; and
—NR⁵R⁶ is morpholinyl, piperazinyl, or piperidinyl, each of which is optionally substituted with one or more substituent groups independently selected from the group consisting of a nitrogen protecting group, alkyl, hydroxy, alkoxy, and alkoxyalkyl.

10. The compound of claim 6 or a pharmaceutically acceptable salt thereof, wherein
R⁹ is phenyl substituted with alkyl, haloalkyl, halo, and/or CN; and
either (i) X is N, and Y and Z are CH, (ii) X and Y are CH, and Z is N, or (iii) X, Y, and Z are each CR¹⁰.

11. The compound of claim 1 or a pharmaceutically acceptable salt thereof, wherein the compound of Formula (I) is a compound Formula (Ic): 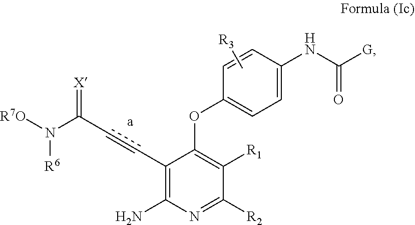
wherein [symbol] is —C≡C— or —CH═CH—.

12. The compound of claim 11 or a pharmaceutically acceptable salt thereof, wherein both R¹ and R² are hydrogen.

13. The compound of claim 11 or a pharmaceutically acceptable salt thereof, wherein R³ is a halo.

14. The compound of claim 11 or a pharmaceutically acceptable salt thereof, wherein
X′ is H₂, (C₁₋₆ alkyl)₂, or ═O;
R⁷ is selected from the group consisting of linear C₁-C₆ alkyl and branched C₃-C₆ alkyl; and
R⁸ is selected from the group consisting of linear C₁-C₆ alkyl and branched C₃-C₆ alkyl.

15. The compound of claim 11 or a pharmaceutically acceptable salt thereof, wherein
R⁹ is phenyl substituted with alkyl, haloalkyl, halo, and/or CN; and
either (i) X is N, and Y and Z are CH, (ii) X and Y are CH, and Z is N, or (iii) X, Y, and Z are each CR¹⁰.

16. A compound of claim 1 selected from 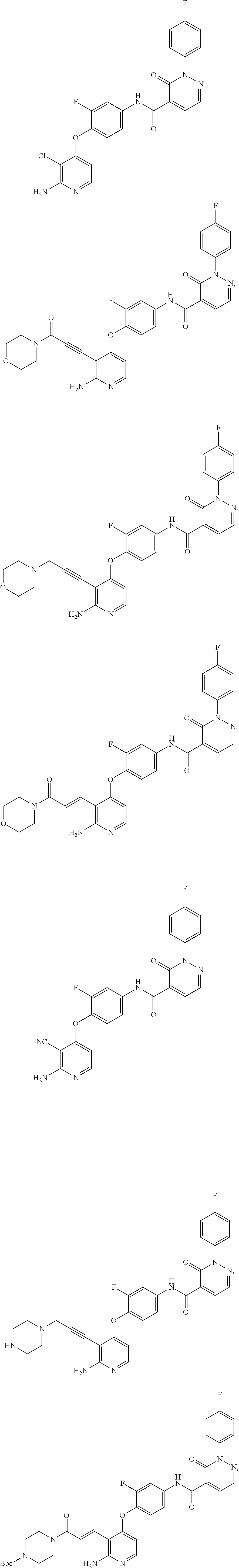 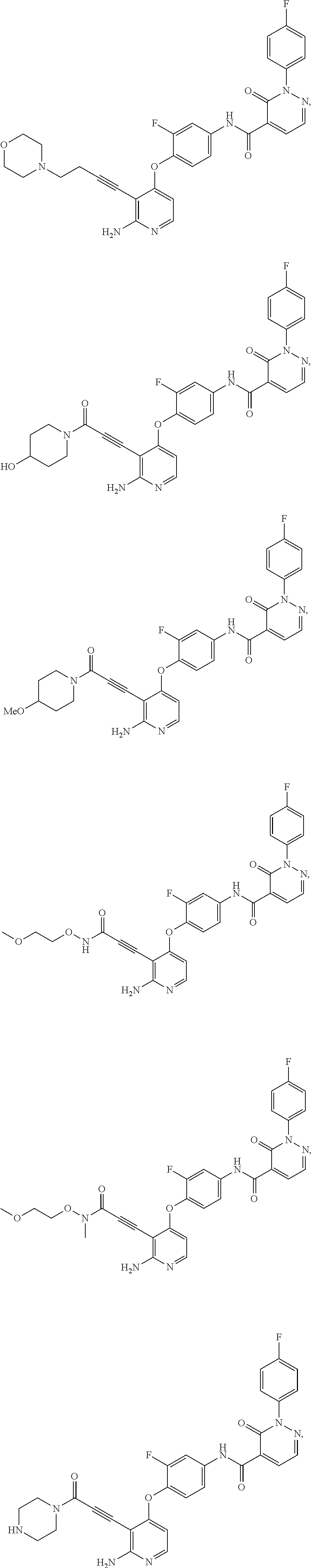 [SMILES: Nc1nccc(Oc2ccc(NC(=O)c3ccnn(-c4ccc(F)cc4)c3=O)cc2F)c1C#CCCN1CCOCC1 ; Nc1nccc(Oc2ccc(NC(=O)c3ccnn(-c4ccc(F)cc4)c3=O)cc2F)c1C#CC(=O)N1CCC(O)CC1 ; COC1CCN(C(=O)C#Cc2c(Oc3ccc(NC(=O)c4ccnn(-c5ccc(F)cc5)c4=O)cc3F)ccnc2N)CC1 ; COCCONC(=O)C#Cc1c(Oc2ccc(NC(=O)c3ccnn(-c4ccc(F)cc4)c3=O)cc2F)ccnc1N ; COCCON(C)C(=O)C#Cc1c(Oc2ccc(NC(=O)c3ccnn(-c4ccc(F)cc4)c3=O)cc2F)ccnc1N ; Nc1nccc(Oc2ccc(NC(=O)c3ccnn(-c4ccc(F)cc4)c3=O)cc2F)c1C#CC(=O)N1CCNCC1] 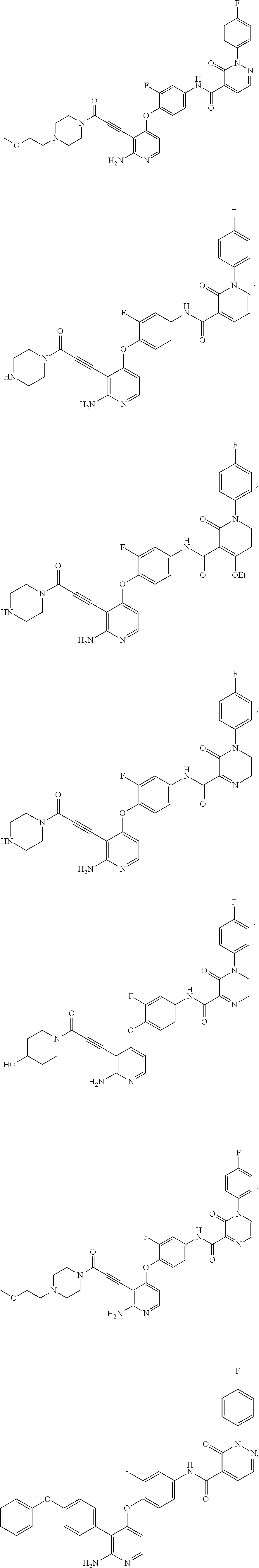 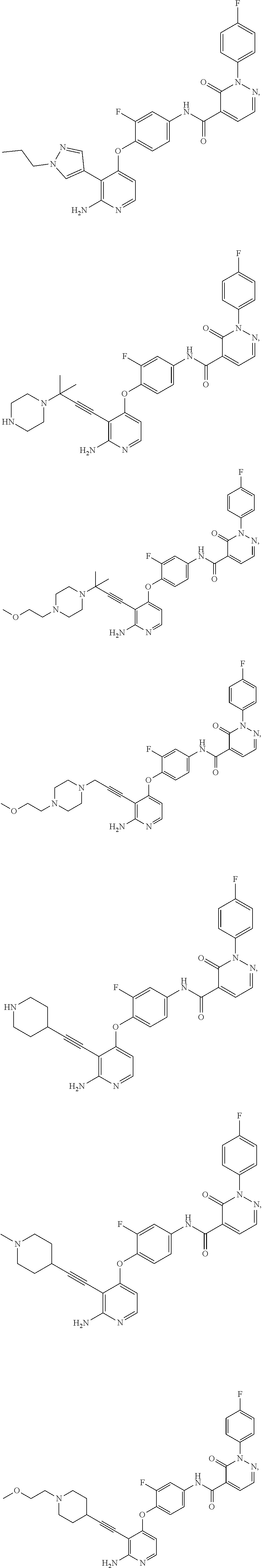 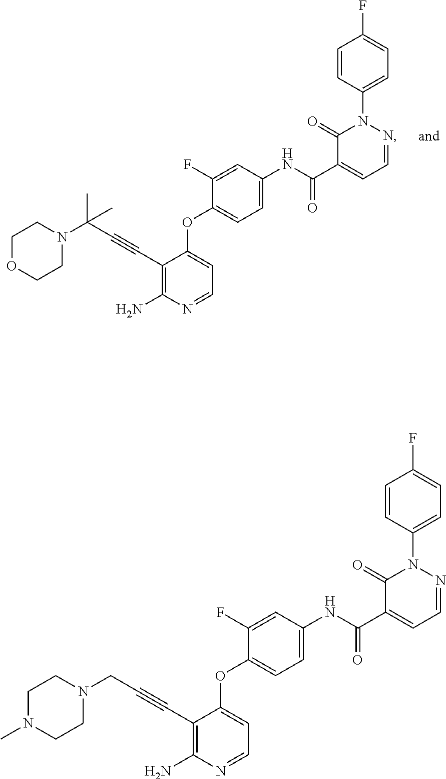 [SMILES: CC(C)(C#Cc1c(Oc2ccc(NC(=O)c3ccnn(-c4ccc(F)cc4)c3=O)cc2F)ccnc1N)N1CCOCC1 ; "and" ; CN1CCN(CC#Cc2c(Oc3ccc(NC(=O)c4ccnn(-c5ccc(F)cc5)c4=O)cc3F)ccnc2N)CC1]
or a pharmaceutically acceptable salt thereof.

17. A pharmaceutical composition comprising at least one compound of claim 1 or a pharmaceutically acceptable salt thereof and a pharmaceutically acceptable carrier.

18. A method of treating or prophylaxis of an AXL-, Mer- and/or c-Met-mediated disease in a subject, wherein the disease is selected from the group consisting of papillary thyroid carcinoma, pancreatic cancer, lung cancer, colon cancer, breast carcinoma, neuroblastoma, pain, cachexia, dermatitis, and asthma, the method comprising administering a pharmaceutically effective amount of the compound of claim 1 or a pharmaceutically acceptable salt thereof to a subject in need of such treatment.

19. The method of claim 18, wherein the lung cancer is non-small cell lung cancer.

20. A method of inhibiting a AXL, Mer, and/or c-Met enzyme in a cell, the method comprising administering a pharmaceutically effective amount of the compound of claim 1 or a pharmaceutically acceptable salt thereof to a cell in need of such inhibition.
